SEQUENCE LISTING (607 sequences)

SEQ ID NO: 1 - DNA, 21 bp, Artificial Sequence
  synthetic oligonucleotide
gaaagagacaaagagagttag

SEQ ID NO: 2 - DNA, 21 bp, Artificial Sequence
  synthetic oligonucleotide
acatctcccctaccgctatag

SEQ ID NO: 3 - DNA, 23 bp, Artificial Sequence
  synthetic oligonucleotide
cagtggatttggaaacagaaatg

SEQ ID NO: 4 - DNA, 23 bp, Artificial Sequence
  synthetic oligonucleotide
tcagtaagttaaaggattgcagg

SEQ ID NO: 5 - DNA, 30 bp, Artificial Sequence
  synthetic oligonucleotide
ccctagtggatgataagaataatcagtatg

SEQ ID NO: 6 - DNA, 35 bp, Artificial Sequence
  synthetic oligonucleotide
ggacagatgataaatacataggataggatggatgg

SEQ ID NO: 7 - DNA, 22 bp, Artificial Sequence
  synthetic oligonucleotide
atatgtgagtcaattccccaag

SEQ ID NO: 8 - DNA, 26 bp, Artificial Sequence
  synthetic oligonucleotide
ggtagatagactggatagatagacga

SEQ ID NO: 9 - DNA, 19 bp, Artificial Sequence
  synthetic oligonucleotide
actgcagtccaatctgggt

SEQ ID NO: 10 - DNA, 20 bp, Artificial Sequence
  synthetic oligonucleotide
atgaaatcaacagaggcttg

SEQ ID NO: 11 - DNA, 21 bp, Artificial Sequence
  synthetic oligonucleotide
atacagacagacagacaggtg

SEQ ID NO: 12 - DNA, 23 bp, Artificial Sequence
  synthetic oligonucleotide
gcatgtatctatcatccatctct

SEQ ID NO: 13 - DNA, 25 bp, Artificial Sequence
  synthetic oligonucleotide
tttttgtatttcatgtgtacattcg

SEQ ID NO: 14 - DNA, 25 bp, Artificial Sequence
  synthetic oligonucleotide
cgtagctataattagttcattttca

SEQ ID NO: 15 - DNA, 27 bp, Artificial Sequence
  synthetic oligonucleotide
gaacacttgtcatagtttagaacgaac

SEQ ID NO: 16 - DNA, 20 bp, Artificial Sequence
  synthetic oligonucleotide
tcattgacagaattgcacca

SEQ ID NO: 17 - DNA, 20 bp, Artificial Sequence
  synthetic oligonucleotide
tctgacccatctaacgccta

SEQ ID NO: 18 - DNA, 27 bp, Artificial Sequence
  synthetic oligonucleotide
cagacagaaagatagatagatgattga

SEQ ID NO: 19 - DNA, 20 bp, Artificial Sequence
  synthetic oligonucleotide
gggtgattttcctctttggt

SEQ ID NO: 20 - DNA, 33 bp, Artificial Sequence
  synthetic oligonucleotide
aacatttgtatctttatctgtatccttatttat

SEQ ID NO: 21 - DNA, 23 bp, Artificial Sequence
  synthetic oligonucleotide
ctgtggctcatctatgaaaactt

SEQ ID NO: 22 - DNA, 22 bp, Artificial Sequence
  synthetic oligonucleotide
gaagtggctgtggtgttatgat

SEQ ID NO: 23 - DNA, 20 bp, Artificial Sequence
  synthetic oligonucleotide
tgcactccagcctgggcaac

SEQ ID NO: 24 - DNA, 22 bp, Artificial Sequence
  synthetic oligonucleotide
ttggtgcacccattacccgaat

SEQ ID NO: 25 - DNA, 23 bp, Artificial Sequence
  synthetic oligonucleotide
ggcatatttacaagctagtttct

SEQ ID NO: 26 - DNA, 20 bp, Artificial Sequence
  synthetic oligonucleotide
atttgtctgtaattgccagc

SEQ ID NO: 27 - DNA, 21 bp, Artificial Sequence
  synthetic oligonucleotide
tgagtgacaaattgagacctt

SEQ ID NO: 28 - DNA, 26 bp, Artificial Sequence
  synthetic oligonucleotide
gtcttacaataacagttgctactatt

SEQ ID NO: 29 - DNA, 23 bp, Artificial Sequence
  synthetic oligonucleotide
acagtaactgccttcatagatag

SEQ ID NO: 30 - DNA, 22 bp, Artificial Sequence
  synthetic oligonucleotide
gtgtcagaccctgttctaagta

SEQ ID NO: 31 - DNA, 22 bp, Artificial Sequence
  synthetic oligonucleotide
gagcaagacaccatctcaagaa

SEQ ID NO: 32 - DNA, 30 bp, Artificial Sequence
  synthetic oligonucleotide
gaaattttacatttatgtttatgattctct

SEQ ID NO: 33 - DNA, 18 bp, Artificial Sequence
  synthetic oligonucleotide
ggcgactgagcaagactc

SEQ ID NO: 34 - DNA, 27 bp, Artificial Sequence
  synthetic oligonucleotide
ggttattaattgagaaaactccttaca

SEQ ID NO: 35 - DNA, 21 bp, Artificial Sequence
  synthetic oligonucleotide
cctgttcctcccttatttccc

SEQ ID NO: 36 - DNA, 22 bp, Artificial Sequence
  synthetic oligonucleotide
gggaacacagactccatggtga

SEQ ID NO: 37 - DNA, 22 bp, Artificial Sequence
  synthetic oligonucleotide
cttagggaaccctcactgaatg

SEQ ID NO: 38 - DNA, 22 bp, Artificial Sequence
  synthetic oligonucleotide
gtccttgtcagcgtttatttgc

SEQ ID NO: 39 - DNA, 25 bp, Artificial Sequence
  synthetic oligonucleotide
ttaatgaattgaacaaatgagtgag

SEQ ID NO: 40 - DNA, 26 bp, Artificial Sequence
  synthetic oligonucleotide
tacaactctggttgtattgtcttcat

SEQ ID NO: 41 - DNA, 23 bp, Artificial Sequence
  synthetic oligonucleotide
attttccccgatgatagtagtct

SEQ ID NO: 42 - DNA, 26 bp, Artificial Sequence
  synthetic oligonucleotide
gcgaatgtatgattggcaatattttt

SEQ ID NO: 43 - DNA, 245 bp, Artificial Sequence
  synthetic oligonucleotide
gaaagagacaaagagagttagaaagaaagaaagagagagagagagaaaggaaggaaggaagaaaaagaaagaaaaagaaagaaagagaaagaaagaaagagaaagaaagaaagaaagaaagaaagaagaaagaaagaaaaagaaagaaagaaagaaagaaagaaagaaagaaagaaagaaagaaagaaagaaagaaagaaaggaargaaagaaagagcaagttactatagcggtaggggagatgt

SEQ ID NO: 44 - DNA, 125 bp, Artificial Sequence
  synthetic oligonucleotide
cagtggatttggaaacagaaatggcttggccttgcctgcctgcctgcctgcctgccttccttccttccttccttccttccttccttccttccttccttccctcctgcaatcctttaacttactga

SEQ ID NO: 45 - DNA, 153 bp, Artificial Sequence
  synthetic oligonucleotide
ccctagtggatgataagaataatcagtatgtgacttggattgatctatctgtctgtctgtctgtctatctatctatctatctatctatctatctatctatctatctatctatctatccatctatccaccatcctatgtatttatcatctgtcc

SEQ ID NO: 46 - DNA, 184 bp, Artificial Sequence
  synthetic oligonucleotide
atatgtgagtcaattccccaagtgaattgccttctatctatctatctatctgtctgtctgtctgtctgtctgtctatctatctatatctatctatctatcatctatctatccatatctatctatctactatctatctatctatctatctatctatctatcgtctatctatccagtctatctacc

SEQ ID NO: 47 - DNA, 127 bp, Artificial Sequence
  synthetic oligonucleotide
actgcagtccaatctgggtgacagagcaagaccctgtctcatagatagatagatagatagatagatagatagatagatagatagacagacagacagatagatacatgcaagcctctgttgatttcat

SEQ ID NO: 48 - DNA, 105 bp, Artificial Sequence
  synthetic oligonucleotide
atacagacagacagacaggtggatagatagatagatagatagatagatagatagatagatagatatcattgaaagacaaamcagagatggatgatagatacatgc

SEQ ID NO: 49 - DNA, 176 bp, Artificial Sequence
  synthetic oligonucleotide
tttttgtatttcatgtgtacattcgtatctatctatctatctatctatctatctatctatctatctatctatctattccccacagtgaaaataatctacaggataggtaaataaattaaggcatattacgcaatgggatacgatacagtgatgaaaatgaactaattatagctacg

SEQ ID NO: 50 - DNA, 168 bp, Artificial Sequence
  synthetic oligonucleotide
gaacacttgtcatagtttagaacgaactaacgatagatagatagatagatagatagatagatagatagatagatagatagatagacagattgatagttttttttwatctcactaaatagtctatagtaaacatttaattaccaatatktggtgcaattctgtcaatga

SEQ ID NO: 51 - DNA, 112 bp, Artificial Sequence
  synthetic oligonucleotide
tctgacccatctaacgcctatctgtatttacaaatacattatctatctatctatctatctatctatctatctatctatctatcwatcaatcatctatctatctttctgtctg

SEQ ID NO: 52 - DNA, 123 bp, Artificial Sequence
  synthetic oligonucleotide
gggtgattttcctctttggtatccttaygtaatattttgaagatagatagatagatagatagatagatagatagatagatagatagaggtataaataaggatacagataaagatacaaatgtt

SEQ ID NO: 53 - DNA, 93 bp, Artificial Sequence
  synthetic oligonucleotide
ctgtggctcatctatgaaaacttctatctatctatctatctatctatctatctatctatctatctatctatatcataacaccacagccacttc

SEQ ID NO: 54 - DNA, 149 bp, Artificial Sequence
  synthetic oligonucleotide
tgcactccagcctgggcaacagaataagattctgttgaaggaaagaaggtaggaaggaaggaaggaaggaaggaaggaaggaaggaaggaaggaaggaaggagagaggaagaaagagagaagattttattcgggtaatgggtgcaccaa

SEQ ID NO: 55 - DNA, 139 bp, Artificial Sequence
  synthetic oligonucleotide
ggcatatttacaagctagtttctttctttcttttttctctttctttctttctttctttctttctttctttctttctttctttctttctttctccttccttcctttcttcctttcttttttgctggcattacagacaaat

SEQ ID NO: 56 - DNA, 156 bp, Artificial Sequence
  synthetic oligonucleotide
tgagtgacaaattgagaccttgtctcagaaagaaagaaagaaagaaagaaagaaagaaagaaagaaagaaagaaagaaagaaagaaagaaagaaagaaaaagagagaggaaagaaagagaaaaagaaagaaatagtagcaactgttattgtaagac

SEQ ID NO: 57 - DNA, 117 bp, Artificial Sequence
  synthetic oligonucleotide
acagtaactgccttcatagatagaagatagatagattagatagatagatagatagatagatagatagatagatagatagatagatagataggaagtacttagaacagggtctgacac

SEQ ID NO: 58 - DNA, 146 bp, Artificial Sequence
  synthetic oligonucleotide
gagcaagacaccatctcaagaaagaaaaaaaagaaagaaaagaaaagaaaagaaaagaaaagaaaagaaaagaaaagaaaagaaaagaaaagaaaagaaaaaacgaaggggaaaaaaagagaatcataacataaatgtaaaatttc

SEQ ID NO: 59 - DNA, 80 bp, Artificial Sequence
  synthetic oligonucleotide
ggcgactgagcaagactcagtctcaaagaaaagaaaagaaaagaaaagaaaattgtaaggagttttctcaattaataacc

SEQ ID NO: 60 - DNA, 79 bp, Artificial Sequence
  synthetic oligonucleotide
cctgttcctcccttatttccctcattcattcattcattcattcattcattcattcattcaccatggagtctgtgttccc

SEQ ID NO: 61 - DNA, 77 bp, Artificial Sequence
  synthetic oligonucleotide
cttagggaaccctcactgaatgaatgaatgaatgaatgaatgaatgaatgtttgggcaaataaacgctgacaaggac

SEQ ID NO: 62 - DNA, 104 bp, Artificial Sequence
  synthetic oligonucleotide
ttaatgaattgaacaaatgagtgagtggaaggaaggaaggaaggaaggaaggaaggaaggaaggaaggaaggaaggaaatgaagacaatacaaccagagttgta

SEQ ID NO: 63 - DNA, 106 bp, Artificial Sequence
  synthetic oligonucleotide
attttccccgatgatagtagtctcattattattattattattattattattattattattattattactattattgttataaaaatattgccaatcatacattcgc

SEQ ID NO: 64 - DNA, 20 bp, Artificial Sequence
  synthetic oligonucleotide
atgccattgcactccaatct

SEQ ID NO: 65 - DNA, 20 bp, Artificial Sequence
  synthetic oligonucleotide
ggcaaggacaaggttctgtg

SEQ ID NO: 66 - DNA, 203 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaaaagaaagaagaaagaaagaaagaaagaaagaaagaaagaaagaaagaaagaaagaaagaaaggaaggaaagaaagagcaagtta

SEQ ID NO: 67 - DNA, 141 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaaggaaagaagaaagagcaagtta

SEQ ID NO: 68 - DNA, 149 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaagaaagaaagaaagagcaagtta

SEQ ID NO: 69 - DNA, 153 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaggaaggaaagaaagagcaagtta

SEQ ID NO: 70 - DNA, 153 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaggaaagaaagaaagagcaagtta

SEQ ID NO: 71 - DNA, 153 bp, Artificial Sequence
  synthetic oligonucleotide
aaagaaagaaagagagagagagagaaaggaaggaaggaagaaaaagaaagaaaaagaaagaaagagaaagaaagagaaagaaagaaagaaagaaagaaagaaagaaagaaagaaagaaagaaagaaaaaaggaaagaaagaaagagcaagtta

SEQ ID NO: 72 - DNA, 154 bp, Artificial Sequence
  synthetic oligonucleotide
aaagaaagaaagagagagagagagaaaggaaggaaggaagaaaaagaaagaaaaagaaagaaagagaaagaaagaaagagaaagaaaagaaagaaagaaagaaagaaagaaagaaagaaagaaagaagaaaggaaagaaagaaagagcaagtta

SEQ ID NO: 73 - DNA, 154 bp, Artificial Sequence; identical to SEQ ID NO: 72
  synthetic oligonucleotide

SEQ ID NO: 74 - DNA, 155 bp, Artificial Sequence
  synthetic oligonucleotide
aaagaaagaaagagagagagagagaaaggaaggaaggaagaaaaagaaagaaaaagaaagaaagagaaagaaagaaagaaagaaagaaagaaagaaagaaagaaagaaagaaagaaagaaagaaagaagaaaggaaagaaagaaagagcaagtta

SEQ ID NO: 75 - DNA, 157 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaaggaaagaaagaaagagcaagtta

SEQ ID NO: 76 - DNA, 157 bp, Artificial Sequence; identical to SEQ ID NO: 75
  synthetic oligonucleotide

SEQ ID NO: 77 - DNA, 157 bp, Artificial Sequence; identical to SEQ ID NO: 75
  synthetic oligonucleotide

SEQ ID NO: 78 - DNA, 157 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaaggaaagaaagagcgagcaagtta

SEQ ID NO: 79 - DNA, 161 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaaggaaagaaagaaagagcaagtta

SEQ ID NO: 80 - DNA, 161 bp, Artificial Sequence; identical to SEQ ID NO: 79
  synthetic oligonucleotide

SEQ ID NO: 81 - DNA, 161 bp, Artificial Sequence; identical to SEQ ID NO: 79
  synthetic oligonucleotide

SEQ ID NO: 82 - DNA, 165 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaaggaaagaaagagcaagtta

SEQ ID NO: 83 - DNA, 165 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaaggaaagaaagaaagagcaagtta

SEQ ID NO: 84 - DNA, 165 bp, Artificial Sequence; identical to SEQ ID NO: 83
  synthetic oligonucleotide

SEQ ID NO: 85 - DNA, 165 bp, Artificial Sequence; identical to SEQ ID NO: 83
  synthetic oligonucleotide

SEQ ID NO: 86 - DNA, 165 bp, Artificial Sequence; identical to SEQ ID NO: 83
  synthetic oligonucleotide

SEQ ID NO: 87 - DNA, 167 bp, Artificial Sequence
  synthetic oligonucleotide
aaagaaagaaagagagagagagagaaaggaaggaaggaagaaaaagaaagaaaaagaaagaaagagaaagaaagaaagaaagaaagaaagaaagaaagaaagaaagaaagaaagaaagaaagaaagaagaaagaaagaaagaaaggaaagaaagaaagagcaagtta

SEQ ID NO: 88 - DNA, 169 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaaggaaagaaagaaagagcaagtta

SEQ ID NO: 89 - DNA, 169 bp, Artificial Sequence; identical to SEQ ID NO: 88
  synthetic oligonucleotide

SEQ ID NO: 90 - DNA, 169 bp, Artificial Sequence; identical to SEQ ID NO: 88
  synthetic oligonucleotide

SEQ ID NO: 91 - DNA, 172 bp, Artificial Sequence
  synthetic oligonucleotide
aaagaaagaaagagagagagagagaaaggaaggaaggaagaaaaagaaagaaaaagaaagaaagagaaagaaagaaagaaagaaagaaagaaagaaagaaagaaagaaagaaagaaagaaagaaagaagaaagaaaggaaagaaagaaaggaaagaaagaaagagcaagtta

SEQ ID NO: 92 - DNA, 173 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaagaaaggaaagaaagaaagagcaagtta

SEQ ID NO: 93 - DNA, 173 bp, Artificial Sequence; identical to SEQ ID NO: 92
  synthetic oligonucleotide

SEQ ID NO: 94 - DNA, 173 bp, Artificial Sequence; identical to SEQ ID NO: 92
  synthetic oligonucleotide

SEQ ID NO: 95 - DNA, 177 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaagaaagaaaggaaagaaagaaagagcaagtta

SEQ ID NO: 96 - DNA, 177 bp, Artificial Sequence; identical to SEQ ID NO: 95
  synthetic oligonucleotide

SEQ ID NO: 97 - DNA, 177 bp, Artificial Sequence; identical to SEQ ID NO: 95
  synthetic oligonucleotide

SEQ ID NO: 98 - DNA, 177 bp, Artificial Sequence; identical to SEQ ID NO: 95
  synthetic oligonucleotide

SEQ ID NO: 99 - DNA, 179 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaaaagaaagaagaaagaaagaaagaaagaaagaaagaaaggaaggaaagaaagagcaagtta

SEQ ID NO: 100 - DNA, 179 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaaaagaagaaagaaagaaagaaagaaagaaagaaaggaaggaaagaaagagcaagtta

SEQ ID NO: 101 - DNA, 181 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaagaaagaaagaaaggaaagaaagaaagagcaagtta

SEQ ID NO: 102 - DNA, 181 bp, Artificial Sequence; identical to SEQ ID NO: 101
  synthetic oligonucleotide

SEQ ID NO: 103 - DNA, 181 bp, Artificial Sequence; identical to SEQ ID NO: 101
  synthetic oligonucleotide

SEQ ID NO: 104 - DNA, 181 bp, Artificial Sequence; identical to SEQ ID NO: 101
  synthetic oligonucleotide

SEQ ID NO: 105 - DNA, 183 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaaggaaggaaagaaagagcaagtta

SEQ ID NO: 106 - DNA, 183 bp, Artificial Sequence; identical to SEQ ID NO: 105
  synthetic oligonucleotide

SEQ ID NO: 107 - DNA, 185 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaagaaagaaagaaagaaaggaaagaaagaaagagcaagtta

SEQ ID NO: 108 - DNA, 185 bp, Artificial Sequence; identical to SEQ ID NO: 107
  synthetic oligonucleotide

SEQ ID NO: 109 - DNA, 185 bp, Artificial Sequence; identical to SEQ ID NO: 107
  synthetic oligonucleotide

SEQ ID NO: 110 - DNA, 185 bp, Artificial Sequence; identical to SEQ ID NO: 107
  synthetic oligonucleotide

SEQ ID NO: 111 - DNA, 187 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaaggaaggaaagaaagagcaagtta

SEQ ID NO: 112 - DNA, 187 bp, Artificial Sequence; identical to SEQ ID NO: 111
  synthetic oligonucleotide

SEQ ID NO: 113 - DNA, 187 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaaggaaggaaagaaagagcaagtta

SEQ ID NO: 114 - DNA, 189 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaagaaagaaagaaagaaagaaaggaaagaaagaaagagcaagtta

SEQ ID NO: 115 - DNA, 189 bp, Artificial Sequence; identical to SEQ ID NO: 114
  synthetic oligonucleotide

SEQ ID NO: 116 - DNA, 189 bp, Artificial Sequence; identical to SEQ ID NO: 114
  synthetic oligonucleotide

SEQ ID NO: 117 - DNA, 191 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaaaagaaagaaagaaagaagaaagaaagaaagaaagaaagaaagaaagaaagaaagaaaggaaggaaagaaagagcaagtta

SEQ ID NO: 118 - DNA, 191 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaaggaaggaaagaaagagcaagtta

SEQ ID NO: 119 - DNA, 193 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaagaaagaaagaaagaaagaaagaaagaaaggaaagaaagaaagagcaagtta

SEQ ID NO: 120 - DNA, 195 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaaggaaggaaagaaagagcaagtta

SEQ ID NO: 121 - DNA, 195 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaaggaaggaaagaaagagcaagtta

SEQ ID NO: 122 - DNA, 195 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaaaagaaagaaagaaagaaagaaagaaagaaagaaagaaaggaaggaaagaaagagcaagtta

SEQ ID NO: 123 - DNA, 199 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaaaagaaagaagaaagaaagaaagaaagaaagaaagaaagaaagaaagaaagaaagaaaggaaggaaagaaagagcaagtta

SEQ ID NO: 124 - DNA, 199 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaagaaaggaaggaaagaaagagcaagtta

SEQ ID NO: 125 - DNA, 199 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaaaagaaagaaagaaagaaagaaagaaagaaagaaagaaagaaaggaaggaaagaaagagcaagtta

SEQ ID NO: 126 - DNA, 203 bp, Artificial Sequence; identical to SEQ ID NO: 66
  synthetic oligonucleotide

SEQ ID NO: 127 - DNA, 203 bp, Artificial Sequence; identical to SEQ ID NO: 66
  synthetic oligonucleotide

SEQ ID NO: 128 - DNA, 203 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaaggaaggaaagaaagagcaagtta

SEQ ID NO: 129 - DNA, 207 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaaaagaaagaaagaagaaagaaagaaagaaagaaagaaagaaagaaagaaagaaagaaagaaagaaagaaaggaaggaaagaaagagcaagtta

SEQ ID NO: 130 - DNA, 207 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaagaaagaaagaaaggaaggaaagaaagagcaagtta

SEQ ID NO: 131 - DNA, 207 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaaggaaggaaagaaagagcaagtta

SEQ ID NO: 132 - DNA, 207 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagagaagaaagaaagaaagaaagaaagaaagaaagaaagaaagaaagaaagaaagaaagaaaggaaggaaagaaagagcaagtta

SEQ ID NO: 133 - DNA, 211 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaaaagaaagaaagaagaaagaaagaaagaaagaaagaaagaaagaaagaaagaaagaaagaaagaaagaaagaaaggaaggaaagaaagagcaagtta

SEQ ID NO: 134 - DNA, 211 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaaaagaagaaagaaagaaagaaagaaagaaagaaagaaagaaagaaagaaagaaagaaagaaagaaaggaaggaaagaaagagcaagtta

SEQ ID NO: 135 - DNA, 211 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaagaaagaaagaaagaaaggaaggaaagaaagagcaagtta

SEQ ID NO: 136 - DNA, 211 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaagaaaggaaggaaagaaagagcaagtta

SEQ ID NO: 137 - DNA, 211 bp, Artificial Sequence; identical to SEQ ID NO: 136
  synthetic oligonucleotide

SEQ ID NO: 138 - DNA, 215 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaaaagaaagaaagaaagaagaaagaaagaaagaaagaaagaaagaaagaaagaaagaaagaaagaaagaaagaaagaaagaaaggaaggaaagaaagagcaagtta

SEQ ID NO: 139 - DNA, 215 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaagaaagaaaggaaggaaagaaagagcaagtta

SEQ ID NO: 140 - DNA, 215 bp, Artificial Sequence; identical to SEQ ID NO: 139
  synthetic oligonucleotide

SEQ ID NO: 141 - DNA, 215 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagagaaagaaagaagaaagaaagaaagaaagaaagaaagaaagaaagaaagaaagaaagaaagaaagaaagaaagaaaggaaggaaagaaagagcaagtta

SEQ ID NO: 142 - DNA, 219 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaagaaagaaagaaagaaagaaagaaaggaaggaaagaaagagcaagtta

SEQ ID NO: 143 - DNA, 219 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaagaaagaaagaaaggaaggaaagaaagagcaagtta

SEQ ID NO: 144 - DNA, 219 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaaaagaaagaaagaaagaaagaaagaaagaaagaaagaaagaaagaaagaaagaaagaaagaaaggaaggaaagaaagagcaagtta

SEQ ID NO: 145 - DNA, 223 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaagaaagaaagaaagaaagaaagaaagaaaggaaggaaagaaagagcaagtta

SEQ ID NO: 146 - DNA, 223 bp, Artificial Sequence; identical to SEQ ID NO: 145
  synthetic oligonucleotide

SEQ ID NO: 147 - DNA, 223 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaagaaagaaagaaagaaaggaaggaaagaaagagcaagtta

SEQ ID NO: 148 - DNA, 223 bp, Artificial Sequence
  synthetic oligonucleotide
aaagaaagaaagagagagagagagaaaggaaggaaggaagaaaaagaaagaaaaagaaagagaaagaaagaaagagaaagaaagaaagaaagaaagaaagaaagaaagaaagaaagaaagaaaaagaagaaagaaagaaagaaagaaagaaagaaagaaagaaagaaagaaagaaagaaagaaagaaagaaagaaagaaaggaaggaaagaaagagcaagtta

SEQ ID NO: 149 - DNA, 223 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaagaaagaaagaaagaaagaaagaaagaaagaaagaaagaaagaaagaaagaaagaaagaaagaaagaaaggaaggaaggaaagaaagagcaagtta

SEQ ID NO: 150 - DNA, 227 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaagaaagaaagaaagaaagaaaggaaggaaagaaagagcaagtta

SEQ ID NO: 151 - DNA, 227 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaaaagaaagaaagaaagaaagaaagaaagaaagaaagaaagaaagaaagaaagaaagaaagaaagaaaggaaggaaagaaagagcaagtta

SEQ ID NO: 152 - DNA, 227 bp, Artificial Sequence; identical to SEQ ID NO: 151
  synthetic oligonucleotide

SEQ ID NO: 153 - DNA, 227 bp, Artificial Sequence
  synthetic oligonucleotide
aaagaaagaaagagagagagagagaaaggaaggaaggaagaaaaagaaagaaaaagaaagagaaagaaagaaagagaaagaaagaaagaaagaaagaaagaaagaaagaaagaaagaaagaaaaagaagaaagaaagaaagaaagaaagaaagaaagaaagaaagaaagaaagaaagaaagaaagaaagaaagaaagaaaggaaggaaggaaagaaagagcaagtta

SEQ ID NO: 154 - DNA, 231 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaaaagaaagaaagaaagaaagaaagaaagaaagaaagaaagaaagaaagaaagaaagaaagaaagaaagaaagaaagaaaggaaggaaagaaagagcaagtta

SEQ ID NO: 155 - DNA, 231 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaagaaaaaagaaagaaagaaaaagaaagaaagaaagaaagaaagaaagaaagaaagaaagaaagaaagaaagaaagaaagaaagaaaggaaggaaagaaagagcaagtta

SEQ ID NO: 156 - DNA, 233 bp, Artificial Sequence
  synthetic oligonucleotide
aaagaaagaaagagagagagagagaaaggaaggaaggaagaaaaagaaagaaaaagaaagaaagagaaagaaagaaagagaaagaaagaaagaaagaaagaaagaaagaaagaaagaaagaaaaagaagaaagaaagaaagaaagaaagaaagaaagaaagaaagaaagaaaaagaaagaaagaaagaaagaaagaaagaaagaaagaaaggaaggaaagaaagagcaagtta

SEQ ID NO: 157 - DNA, 20 bp, Artificial Sequence
  synthetic oligonucleotide
cataatccagctgtgggagg

SEQ ID NO: 158 - DNA, 23 bp, Artificial Sequence
  synthetic oligonucleotide
ttcatgcctacatccctagtacc

SEQ ID NO: 159 - DNA, 79 bp, Artificial Sequence
  synthetic oligonucleotide
gcttggccttgcctgcctgcctgcctgcctgccttccttccttccttccttccttccttccttccttccttccttccct

SEQ ID NO: 160 - DNA, 75 bp, Artificial Sequence
  synthetic oligonucleotide
gcttggccttgcctgcctgcctgcctgcctgccttccttccttccttccttccttccttccttccttccttccct

SEQ ID NO: 161 - DNA, 75 bp, Artificial Sequence; identical to SEQ ID NO: 160
  synthetic oligonucleotide

SEQ ID NO: 162 - DNA, 79 bp, Artificial Sequence; identical to SEQ ID NO: 159
  synthetic oligonucleotide

SEQ ID NO: 163 - DNA, 79 bp, Artificial Sequence; identical to SEQ ID NO: 159
  synthetic oligonucleotide

SEQ ID NO: 164 - DNA, 79 bp, Artificial Sequence; identical to SEQ ID NO: 159
  synthetic oligonucleotide

SEQ ID NO: 165 - DNA, 79 bp, Artificial Sequence; identical to SEQ ID NO: 159
  synthetic oligonucleotide

SEQ ID NO: 166 - DNA, 79 bp, Artificial Sequence; identical to SEQ ID NO: 159
  synthetic oligonucleotide

SEQ ID NO: 167 - DNA, 79 bp, Artificial Sequence; identical to SEQ ID NO: 159
  synthetic oligonucleotide

SEQ ID NO: 168 - DNA, 83 bp, Artificial Sequence
  synthetic oligonucleotide
gcttggccttgcctgcctgcctgcctgcctgccttccttccttccttccttccttccttccttccttccttccttccttccct

SEQ ID NO: 169 - DNA, 83 bp, Artificial Sequence; identical to SEQ ID NO: 168
  synthetic oligonucleotide

SEQ ID NO: 170 - DNA, 83 bp, Artificial Sequence; identical to SEQ ID NO: 168
  synthetic oligonucleotide

SEQ ID NO: 171 - DNA, 83 bp, Artificial Sequence; identical to SEQ ID NO: 168
  synthetic oligonucleotide

SEQ ID NO: 172 - DNA, 83 bp, Artificial Sequence
  synthetic oligonucleotide
gcttggccttgcctgcctgcctgcctgcctgcctgccttccttccttccttccttccttccttccttccttccttccttccct

SEQ ID NO: 173 - DNA, 83 bp, Artificial Sequence; identical to SEQ ID NO: 172
  synthetic oligonucleotide

SEQ ID NO: 174 - DNA, 83 bp, Artificial Sequence; identical to SEQ ID NO: 172
  synthetic oligonucleotide

SEQ ID NO: 175 - DNA, 87 bp, Artificial Sequence
  synthetic oligonucleotide
gcttggccttgcctgcctgcctgcctgcctgccttccttccttccttccttccttccttccttccttccttccttccttccttccct

SEQ ID NO: 176 - DNA, 87 bp, Artificial Sequence; identical to SEQ ID NO: 175
  synthetic oligonucleotide

SEQ ID NO: 177 - DNA, 87 bp, Artificial Sequence; identical to SEQ ID NO: 175
  synthetic oligonucleotide

SEQ ID NO: 178 - DNA, 87 bp, Artificial Sequence
  synthetic oligonucleotide
gcttggccttgcctgcctgcctgcctgcctgcctgccttccttccttccttccttccttccttccttccttccttccttccttccct

SEQ ID NO: 179 - DNA, 87 bp, Artificial Sequence; identical to SEQ ID NO: 178
  synthetic oligonucleotide

SEQ ID NO: 180 - DNA, 87 bp, Artificial Sequence; identical to SEQ ID NO: 178
  synthetic oligonucleotide

SEQ ID NO: 181 - DNA, 87 bp, Artificial Sequence; identical to SEQ ID NO: 178
  synthetic oligonucleotide

SEQ ID NO: 182 - DNA, 91 bp, Artificial Sequence
  synthetic oligonucleotide
gcttggccttgcctgcctgcctgcctgcctgccttccttccttccttccttccttccttccttccttccttccttccttccttccttccct

SEQ ID NO: 183 - DNA, 91 bp, Artificial Sequence
  synthetic oligonucleotide
gcttggccttgcctgcctgcctgcctgcctgcctgccttccttccttccttccttccttccttccttccttccttccttccttccttccct

SEQ ID NO: 184 - DNA, 91 bp, Artificial Sequence; identical to SEQ ID NO: 183
  synthetic oligonucleotide

SEQ ID NO: 185 - DNA, 91 bp, Artificial Sequence; identical to SEQ ID NO: 183
  synthetic oligonucleotide

SEQ ID NO: 186 - DNA, 91 bp, Artificial Sequence; identical to SEQ ID NO: 183
  synthetic oligonucleotide

SEQ ID NO: 187 - DNA, 91 bp, Artificial Sequence; identical to SEQ ID NO: 183
  synthetic oligonucleotide

SEQ ID NO: 188 - DNA, 91 bp, Artificial Sequence
  synthetic oligonucleotide
gcttggccttgcctgcctgcctgcctgcctgcctgccttccttccttccttccttccttccttccttccttccttccgtccttccttccct

SEQ ID NO: 189 - DNA, 91 bp, Artificial Sequence; identical to SEQ ID NO: 188
  synthetic oligonucleotide

SEQ ID NO: 190 - DNA, 91 bp, Artificial Sequence; identical to SEQ ID NO: 188
  synthetic oligonucleotide

SEQ ID NO: 191 - DNA, 91 bp, Artificial Sequence
  synthetic oligonucleotide
gcttggccttgcctgcctgcctgcctgcctgcctgcctgccttccttccttccttccttccttccttccttccttccttccttccttccct

SEQ ID NO: 192 - DNA, 91 bp, Artificial Sequence
  synthetic oligonucleotide
gcttggccttgcctgcctgcctgcctgcctgcctgcctcccttccttccttccttccttccttccttccttccttccttccttccttccct

SEQ ID NO: 193 - DNA, 95 bp, Artificial Sequence
  synthetic oligonucleotide
gcttggccttgcctgcctgcctgcctgcctgcctgccttccttccttccttccttccttccttccttccttccttccttccttccttccttccct

SEQ ID NO: 194 - DNA, 95 bp, Artificial Sequence; identical to SEQ ID NO: 193
  synthetic oligonucleotide

SEQ ID NO: 195 - DNA, 95 bp, Artificial Sequence; identical to SEQ ID NO: 193
  synthetic oligonucleotide

SEQ ID NO: 196 - DNA, 99 bp, Artificial Sequence
  synthetic oligonucleotide
gcttggccttgcctgcctgcctgcctgcctgcctgccttccttccttccttccttccttccttccttccttccttccttccttccttccttccttccct

SEQ ID NO: 197 - DNA, 99 bp, Artificial Sequence; identical to SEQ ID NO: 196
  synthetic oligonucleotide

SEQ ID NO: 198 - DNA, 99 bp, Artificial Sequence
  synthetic oligonucleotide
gcttggccttgcctgcctgcctgcctgcctgcctgccttccttccttccttccttccttccttccttccttccttccttccttccgtccttccttccct

SEQ ID NO: 199 - DNA, 99 bp, Artificial Sequence; identical to SEQ ID NO: 198
  synthetic oligonucleotide

SEQ ID NO: 200 - DNA, 99 bp, Artificial Sequence; identical to SEQ ID NO: 198
  synthetic oligonucleotide

SEQ ID NO: 201 - DNA, 99 bp, Artificial Sequence; identical to SEQ ID NO: 198
  synthetic oligonucleotide

SEQ ID NO: 202 - DNA, 99 bp, Artificial Sequence; identical to SEQ ID NO: 198
  synthetic oligonucleotide

SEQ ID NO: 203 - DNA, 103 bp, Artificial Sequence
  synthetic oligonucleotide
gcttggccttgcctgcctgcctgcctgcctgccttccttccttccttccttccttccttccttccttccttccttccttccttccttccgtccttccttccct

SEQ ID NO: 204 - DNA, 103 bp, Artificial Sequence
  synthetic oligonucleotide
gcttggccttgcctgcctgcctgcctgcctgcctgccttccttccttccttccttccttccttccttccttccttccttccttccttccgtccttccttccct

SEQ ID NO: 205 - DNA, 103 bp, Artificial Sequence; identical to SEQ ID NO: 204
  synthetic oligonucleotide

SEQ ID NO: 206 - DNA, 103 bp, Artificial Sequence; identical to SEQ ID NO: 204
  synthetic oligonucleotide

SEQ ID NO: 207 - DNA, 103 bp, Artificial Sequence; identical to SEQ ID NO: 204
  synthetic oligonucleotide

SEQ ID NO: 208 - DNA, 107 bp, Artificial Sequence
  synthetic oligonucleotide
gcttggccttgcctgcctgcctgcctgcctgcctgccttccttccttccttccttccttccttccttccttccttccttccttccttccttccgtccttccttccct

SEQ ID NO: 209 - DNA, 107 bp, Artificial Sequence; identical to SEQ ID NO: 208
  synthetic oligonucleotide

SEQ ID NO: 210 - DNA, 107 bp, Artificial Sequence; identical to SEQ ID NO: 208
  synthetic oligonucleotide

SEQ ID NO: 211 - DNA, 107 bp, Artificial Sequence; identical to SEQ ID NO: 208
  synthetic oligonucleotide

SEQ ID NO: 212 - DNA, 107 bp, Artificial Sequence; identical to SEQ ID NO: 208
  synthetic oligonucleotide

SEQ ID NO: 213 - DNA, 107 bp, Artificial Sequence
  synthetic oligonucleotide
gcttggccttgcctgcctgcctgcctgcctgcctgcctgccttccttccttccttccttccttccttccttccttccttccttccttccttccgtccttccttccct

SEQ ID NO: 214 - DNA, 107 bp, Artificial Sequence; identical to SEQ ID NO: 213
  synthetic oligonucleotide

SEQ ID NO: 215 - DNA, 111 bp, Artificial Sequence
  synthetic oligonucleotide
gcttggccttgcctgcctgcctgcctgcctgcctgccttccttccttccttccttccttccttccttccttccttccttccttccttccttccttccgtccttccttccct

SEQ ID NO: 216 - DNA, 111 bp, Artificial Sequence; identical to SEQ ID NO: 215
  synthetic oligonucleotide

SEQ ID NO: 217 - DNA, 111 bp, Artificial Sequence; identical to SEQ ID NO: 215
  synthetic oligonucleotide

SEQ ID NO: 218 - DNA, 111 bp, Artificial Sequence; identical to SEQ ID NO: 215
  synthetic oligonucleotide

SEQ ID NO: 219 - DNA, 111 bp, Artificial Sequence; identical to SEQ ID NO: 215
  synthetic oligonucleotide

SEQ ID NO: 220 - DNA, 111 bp, Artificial Sequence
  synthetic oligonucleotide
gcttggccttgcctgcctgcctgcctgcctgcctgcctgccttccttccttccttccttccttccttccttccttccttccttccttccttccttccgtccttccttccct

SEQ ID NO: 221 - DNA, 111 bp, Artificial Sequence; identical to SEQ ID NO: 220
  synthetic oligonucleotide

SEQ ID NO: 222 - DNA, 111 bp, Artificial Sequence; identical to SEQ ID NO: 220
  synthetic oligonucleotide

SEQ ID NO: 223 - DNA, 111 bp, Artificial Sequence; identical to SEQ ID NO: 220
  synthetic oligonucleotide

SEQ ID NO: 224 - DNA, 115 bp, Artificial Sequence
  synthetic oligonucleotide
gcttggccttgcctgcctgcctgcctgcctgcctgccttccttccttccttccttccttccttccttccttccttccttccttccttccttccttccttccgtccttccttccct

SEQ ID NO: 225 - DNA, 115 bp, Artificial Sequence; identical to SEQ ID NO: 224
  synthetic oligonucleotide

SEQ ID NO: 226 - DNA, 20 bp, Artificial Sequence
  synthetic oligonucleotide
tgtgaaagccctagtggatg

SEQ ID NO: 227 - DNA, 27 bp, Artificial Sequence
  synthetic oligonucleotide
acaaaggatagatagagacaggataga

SEQ ID NO: 228 - DNA, 94 bp, Artificial Sequence
  synthetic oligonucleotide
tgacttggattgatctatctgtctgtctgtctgtctatctatctatctatctatctatctatctatctatctatctatctatctatccatctat

SEQ ID NO: 229 - DNA, 78 bp, Artificial Sequence
  synthetic oligonucleotide
tgacttggattgatctatctgtctatctgtctgtctgtctgtctatctatctatccatctatctatctatccatccat

SEQ ID NO: 230 - DNA, 78 bp, Artificial Sequence; identical to SEQ ID NO: 229
  synthetic oligonucleotide

SEQ ID NO: 231 - DNA, 78 bp, Artificial Sequence; identical to SEQ ID NO: 229
  synthetic oligonucleotide

SEQ ID NO: 232 - DNA, 78 bp, Artificial Sequence; identical to SEQ ID NO: 229
  synthetic oligonucleotide

SEQ ID NO: 233 - DNA, 78 bp, Artificial Sequence; identical to SEQ ID NO: 229
  synthetic oligonucleotide

SEQ ID NO: 234 - DNA, 78 bp, Artificial Sequence; identical to SEQ ID NO: 229
  synthetic oligonucleotide

SEQ ID NO: 235 - DNA, 78 bp, Artificial Sequence
  synthetic oligonucleotide
tgacttggattgatctatctgtctgtctgtctatctatctatctatctatctatctatctatctatctatccatctat

SEQ ID NO: 236 - DNA, 78 bp, Artificial Sequence; identical to SEQ ID NO: 235
  synthetic oligonucleotide

SEQ ID NO: 237 - DNA, 82 bp, Artificial Sequence
  synthetic oligonucleotide
tgacttggattgatctatctgtctgtctgtctgtctatctatctatctatctatctatctatctatctatctatccatctat

SEQ ID NO: 238 - DNA, 82 bp, Artificial Sequence; identical to SEQ ID NO: 237
  synthetic oligonucleotide

SEQ ID NO: 239 - DNA, 82 bp, Artificial Sequence; identical to SEQ ID NO: 237
  synthetic oligonucleotide

SEQ ID NO: 240 - DNA, 82 bp, Artificial Sequence; identical to SEQ ID NO: 237
  synthetic oligonucleotide

SEQ ID NO: 241 - DNA, 82 bp, Artificial Sequence
  synthetic oligonucleotide
tgacttggattgatctatctgtctgtctgtctatctatctatctatctatctatctatctatctatctatctatccatctat

SEQ ID NO: 242 - DNA, 82 bp, Artificial Sequence; identical to SEQ ID NO: 241
  synthetic oligonucleotide

SEQ ID NO: 243 - DNA, 82 bp, Artificial Sequence; identical to SEQ ID NO: 241
  synthetic oligonucleotide

SEQ ID NO: 244 - DNA, 82 bp, Artificial Sequence; identical to SEQ ID NO: 241
  synthetic oligonucleotide

SEQ ID NO: 245 - DNA, 82 bp, Artificial Sequence; identical to SEQ ID NO: 241
  synthetic oligonucleotide

SEQ ID NO: 246 - DNA, 82 bp, Artificial Sequence; identical to SEQ ID NO: 241
  synthetic oligonucleotide

SEQ ID NO: 247 - DNA, 82 bp, Artificial Sequence; identical to SEQ ID NO: 241
  synthetic oligonucleotide

SEQ ID NO: 248 - DNA, 86 bp, Artificial Sequence
  synthetic oligonucleotide
tgacttggattgatctatctgtctgtctgtctgtctatctatctatctatctatctatctatctatctatctatctatccatctat

SEQ ID NO: 249 - DNA, 86 bp, Artificial Sequence; identical to SEQ ID NO: 248
  synthetic oligonucleotide

SEQ ID NO: 250 - DNA, 86 bp, Artificial Sequence; identical to SEQ ID NO: 248
  synthetic oligonucleotide

SEQ ID NO: 251 - DNA, 86 bp, Artificial Sequence; identical to SEQ ID NO: 248
  synthetic oligonucleotide

SEQ ID NO: 252 - DNA, 86 bp, Artificial Sequence; identical to SEQ ID NO: 248
  synthetic oligonucleotide

SEQ ID NO: 253 - DNA, 86 bp, Artificial Sequence; identical to SEQ ID NO: 248
  synthetic oligonucleotide

SEQ ID NO: 254 - DNA, 86 bp, Artificial Sequence; identical to SEQ ID NO: 248
  synthetic oligonucleotide

SEQ ID NO: 255 - DNA, 86 bp, Artificial Sequence
  synthetic oligonucleotide
tgacttggattgatctatctgtctgtctgtctatctatctatctatctatctatctatctatctatctatctatctatccatctat

SEQ ID NO: 256 - DNA, 86 bp, Artificial Sequence; identical to SEQ ID NO: 255
  synthetic oligonucleotide

SEQ ID NO: 257 - DNA, 86 bp, Artificial Sequence; identical to SEQ ID NO: 255
  synthetic oligonucleotide

SEQ ID NO: 258 - DNA, 90 bp, Artificial Sequence
  synthetic oligonucleotide
tgacttggattgatctatctgtctgtctgtctgtctatctatctatctatctatctatctatctatctatctatctatctatccatctat

SEQ ID NO: 259 - DNA, 90 bp, Artificial Sequence; identical to SEQ ID NO: 258
  synthetic oligonucleotide

SEQ ID NO: 260 - DNA, 90 bp, Artificial Sequence; identical to SEQ ID NO: 258
  synthetic oligonucleotide

SEQ ID NO: 261 - DNA, 90 bp, Artificial Sequence; identical to SEQ ID NO: 258
  synthetic oligonucleotide

SEQ ID NO: 262 - DNA, 90 bp, Artificial Sequence; identical to SEQ ID NO: 258
  synthetic oligonucleotide

SEQ ID NO: 263 - DNA, 90 bp, Artificial Sequence
  synthetic oligonucleotide
tgacttggattgatctatctgtctgtctgtctatctatctatctatctatctatctatctatctatctatctatctatctatccatctat

SEQ ID NO: 264 - DNA, 90 bp, Artificial Sequence; identical to SEQ ID NO: 263
  synthetic oligonucleotide

SEQ ID NO: 265 - DNA, 94 bp, Artificial Sequence; identical to SEQ ID NO: 228
  synthetic oligonucleotide

SEQ ID NO: 266 - DNA, 94 bp, Artificial Sequence; identical to SEQ ID NO: 228
  synthetic oligonucleotide

SEQ ID NO: 267 - DNA, 94 bp, Artificial Sequence; identical to SEQ ID NO: 228
  synthetic oligonucleotide

SEQ ID NO: 268 - DNA, 94 bp, Artificial Sequence; identical to SEQ ID NO: 228
  synthetic oligonucleotide

SEQ ID NO: 269 - DNA, 94 bp, Artificial Sequence
  synthetic oligonucleotide
tgacttggattgatctatctgtctgtctgtctatctatctatctatctatctatctatctatctatctatctatctatctatctatccatctat

SEQ ID NO: 270 - DNA, 94 bp, Artificial Sequence; identical to SEQ ID NO: 269
  synthetic oligonucleotide

SEQ ID NO: 271 - DNA, 94 bp, Artificial Sequence
  synthetic oligonucleotide
tgacttggattgaactatctgtctgtctgtctgtctatctatctatctatctatctatctatctatctatctatctatctatctatccatctat

SEQ ID NO: 272 - DNA, 98 bp, Artificial Sequence
  synthetic oligonucleotide
tgacttggattgatctatctgtctgtctgtctgtctatctatctatctatctatctatctatctatctatctatctatctatctatctatccatctat

SEQ ID NO: 273 - DNA, 98 bp, Artificial Sequence; identical to SEQ ID NO: 272
  synthetic oligonucleotide

SEQ ID NO: 274 - DNA, 98 bp, Artificial Sequence; identical to SEQ ID NO: 272
  synthetic oligonucleotide

SEQ ID NO: 275 - DNA, 98 bp, Artificial Sequence; identical to SEQ ID NO: 272
  synthetic oligonucleotide

SEQ ID NO: 276 - DNA, 98 bp, Artificial Sequence; identical to SEQ ID NO: 272
  synthetic oligonucleotide

SEQ ID NO: 277 - DNA, 98 bp, Artificial Sequence; identical to SEQ ID NO: 272
  synthetic oligonucleotide

SEQ ID NO: 278 - DNA, 98 bp, Artificial Sequence; identical to SEQ ID NO: 272
  synthetic oligonucleotide

SEQ ID NO: 279 - DNA, 98 bp, Artificial Sequence
  synthetic oligonucleotide
tgacttggattgatctatctgtctgtctgtctgtctgtctatctatctatctatctatctatctatctatctatctatctatctatctatccatctat

SEQ ID NO: 280 - DNA, 98 bp, Artificial Sequence
  synthetic oligonucleotide
tgacttggattgatctatctgtctgtctgtctatctatctatctatctatctatctatctatctatctatctatctatctatctatctatccatctat

SEQ ID NO: 281 - DNA, 98 bp, Artificial Sequence
  synthetic oligonucleotide
tgacttggattgaactatctgtctgtctgtctgtctatctatctatctatctatctatctatctatctatctatctatctatctatctatccatctat

SEQ ID NO: 282 - DNA, 102 bp, Artificial Sequence
  synthetic oligonucleotide
tgacttggattgatctatctgtctgtctgtctgtctatctatctatctatctatctatctatctatctatctatctatctatctatctatctatccatctat

SEQ ID NO: 283 - DNA, 22 bp, Artificial Sequence
  synthetic oligonucleotide
tgggacttttctcagtctccat

SEQ ID NO: 284 - DNA, 24 bp, Artificial Sequence
  synthetic oligonucleotide
agtcaatgttctccagagacagac

SEQ ID NO: 285 - DNA, 174 bp, Artificial Sequence
  synthetic oligonucleotide
tgaattgccttctatctatctatctatctgtctgtctgtctgtctgtctgtctatctatctatatctatctatctatcatctatctatccatatctatctatctatctatctatctatctatctatcatctatctatcgtctatctatccagtctatctacctcctattagtct

SEQ ID NO: 286 - DNA, 166 bp, Artificial Sequence
  synthetic oligonucleotide
tgaattgccttctatctatctatctatctgtctgtctgtctgtctgtctgtctatctatctatatctatctatctatcatctatctatccatatctatctatctatctatctatctatctatctatcatcgtctatctatccagtctatctacctcctattagtct

SEQ ID NO: 287 - DNA, 166 bp, Artificial Sequence; identical to SEQ ID NO: 286
  synthetic oligonucleotide

SEQ ID NO: 288 - DNA, 166 bp, Artificial Sequence; identical to SEQ ID NO: 286
  synthetic oligonucleotide

SEQ ID NO: 289 - DNA, 166 bp, Artificial Sequence; identical to SEQ ID NO: 286
  synthetic oligonucleotide

SEQ ID NO: 290 - DNA, 170 bp, Artificial Sequence
  synthetic oligonucleotide
tgaattgccttctatctatctatctatctgtctgtctgtctgtctgtctgtctatctatctatatctatctatctatcatctatctatccatatctatctatctatctatctatctatctatctatcatctatcgtctatctatccagtctatctacctcctattagtct

SEQ ID NO: 291 - DNA, 170 bp, Artificial Sequence; identical to SEQ ID NO: 290
  synthetic oligonucleotide

SEQ ID NO: 292 - DNA, 170 bp, Artificial Sequence; identical to SEQ ID NO: 290
  synthetic oligonucleotide

SEQ ID NO: 293 - DNA, 170 bp, Artificial Sequence; identical to SEQ ID NO: 290
  synthetic oligonucleotide

SEQ ID NO: 294 - DNA, 174 bp, Artificial Sequence; identical to SEQ ID NO: 285
  synthetic oligonucleotide

SEQ ID NO: 295 - DNA, 174 bp, Artificial Sequence; identical to SEQ ID NO: 285
  synthetic oligonucleotide

SEQ ID NO: 296 - DNA, 174 bp, Artificial Sequence; identical to SEQ ID NO: 285
  synthetic oligonucleotide

SEQ ID NO: 297 - DNA, 174 bp, Artificial Sequence; identical to SEQ ID NO: 285
  synthetic oligonucleotide

SEQ ID NO: 298 - DNA, 174 bp, Artificial Sequence
  synthetic oligonucleotide
tgaattgccttctatctatctatctatctatctatctgtctgtctgtctgtctgtctatctatctatatctatctatctatcatctatctatccatatctatctatctatctatctatctatctatcatctatctatcgtctatctatccagtctatctacctcctattagtct

SEQ ID NO: 299 - DNA, 174 bp, Artificial Sequence; identical to SEQ ID NO: 298
  synthetic oligonucleotide

SEQ ID NO: 300 - DNA, 174 bp, Artificial Sequence; identical to SEQ ID NO: 298
  synthetic oligonucleotide

SEQ ID NO: 301 - DNA, 174 bp, Artificial Sequence; identical to SEQ ID NO: 298
  synthetic oligonucleotide

SEQ ID NO: 302 - DNA, 178 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatctatcgtctatctatccagtctatctacctcctattagtct

SEQ ID NO: 303 - DNA, 178 bp, Artificial Sequence; identical to SEQ ID NO: 302
  synthetic oligonucleotide

SEQ ID NO: 304 - DNA, 178 bp, Artificial Sequence
  synthetic oligonucleotide
tgaattgccttctatctatctatctatctgtctgtctgtctgtctgtctgtctatctatctatatctatctatctatcatctatctatccatatctatctatctatctatctatctatctatctatcatctatctatctatcgtctatctatccagtctatctacctcctattagtct

SEQ ID NO: 305 - DNA, 178 bp, Artificial Sequence
  synthetic oligonucleotide
tgaattgccttctatctatctatctatctatctatctgtctgtctgtctgtctgtctatctatctatatctatctatctatcatctatctatccatatctatctatctatctatctatctatctatcatctatctatctatcgtctatctatccagtctatctacctcctattagtct

SEQ ID NO: 306 - DNA, 178 bp, Artificial Sequence; identical to SEQ ID NO: 305
  synthetic oligonucleotide

SEQ ID NO: 307 - DNA, 178 bp, Artificial Sequence; identical to SEQ ID NO: 305
  synthetic oligonucleotide

SEQ ID NO: 308 - DNA, 178 bp, Artificial Sequence; identical to SEQ ID NO: 305
  synthetic oligonucleotide

SEQ ID NO: 309 - DNA, 180 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatatctatcgtctatctatccagtctatctacctcctattagtct

SEQ ID NO: 310 - DNA, 180 bp, Artificial Sequence; identical to SEQ ID NO: 309
  synthetic oligonucleotide

SEQ ID NO: 311 - DNA, 180 bp, Artificial Sequence; identical to SEQ ID NO: 309
  synthetic oligonucleotide

SEQ ID NO: 312 - DNA, 180 bp, Artificial Sequence
  synthetic oligonucleotide
tgaattgccttctatctatctatctatctatctgtctgtctgtctgtctgtctatctatctatatctatctatctatcatctatctatccatatctatctatctatctatctatctatctatctatcatctatctatatctatcgtctatctatccagtctatctacctcctattagtct

SEQ ID NO: 313 - DNA, 182 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatctatctatcgtctatctatccagtctatctacctcctattagtct

SEQ ID NO: 314 - DNA, 182 bp, Artificial Sequence
  synthetic oligonucleotide
tgaattgccttctatctatctatctatctgtctgtctgtctgtctgtctgtctatctatctatatctatctatctatcatctatctatccatatctatctatctatctatctatctatctatctatcatctatctatctatctatcgtctatctatccagtctatctacctcctattagtct

SEQ ID NO: 315 - DNA, 182 bp, Artificial Sequence
  synthetic oligonucleotide
tgaattgccttctatctatctatctatctatctatctgtctgtctgtctgtctgtctatctatctatatctatctatctatcatctatctatccatatctatctatctatctatctatctatctatcatctatctatctatctatcgtctatctatccagtctatctacctcctattagtct

SEQ ID NO: 316 - DNA, 182 bp, Artificial Sequence; identical to SEQ ID NO: 315
  synthetic oligonucleotide

SEQ ID NO: 317 - DNA, 182 bp, Artificial Sequence; identical to SEQ ID NO: 315
  synthetic oligonucleotide

SEQ ID NO: 318 - DNA, 184 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatctatatctatcgtctatctatccagtctatctacctcctattagtct

SEQ ID NO: 319 - DNA, 184 bp, Artificial Sequence; identical to SEQ ID NO: 318
  synthetic oligonucleotide

SEQ ID NO: 320 - DNA, 184 bp, Artificial Sequence; identical to SEQ ID NO: 318
  synthetic oligonucleotide

SEQ ID NO: 321 - DNA, 186 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatctatctatctatcgtctatctatccagtctatctacctcctattagtct

SEQ ID NO: 322 - DNA, 186 bp, Artificial Sequence
  synthetic oligonucleotide
tgaattgccttctatctatctatctatctgtctgtctgtctgtctgtctgtctatctatctatatctatctatctatcatctatctatccatatctatctatctatctatctatctatctatctatcatctatctatctatctatctatcgtctatctatccagtctatctacctcctattagtct

SEQ ID NO: 323 - DNA, 186 bp, Artificial Sequence
  synthetic oligonucleotide
tgaattgccttctatctatctatctatctatctatctgtctgtctgtctgtctgtctatctatctatatctatctatctatcatctatctatccatatctatctatctatctatctatctatctatcatctatctatctatctatctatcgtctatctatccagtctatctacctcctattagtct

SEQ ID NO: 324 - DNA, 188 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatctatctatatctatcgtctatctatccagtctatctacctcctattagtct

SEQ ID NO: 325 - DNA, 188 bp, Artificial Sequence; identical to SEQ ID NO: 324
  synthetic oligonucleotide

SEQ ID NO: 326 - DNA, 188 bp, Artificial Sequence; identical to SEQ ID NO: 324
  synthetic oligonucleotide

SEQ ID NO: 327 - DNA, 188 bp, Artificial Sequence; identical to SEQ ID NO: 324
  synthetic oligonucleotide

SEQ ID NO: 328 - DNA, 192 bp, Artificial Sequence
  synthetic oligonucleotide
tgaattgccttctatctatctatctatctatctgtctgtctgtctgtctgtctgtctatctatctatatctatctatctatcatctatctatccatatctatctatctatctatctatctatctatcatctatctatctatctatctatatctatcgtctatctatccagtctatctacctcctattagtct

SEQ ID NO: 329 - DNA, 192 bp, Artificial Sequence; identical to SEQ ID NO: 328
  synthetic oligonucleotide

SEQ ID NO: 330 - DNA, 192 bp, Artificial Sequence; identical to SEQ ID NO: 328
  synthetic oligonucleotide

SEQ ID NO: 331 - DNA, 196 bp, Artificial Sequence
  synthetic oligonucleotide
tgaattgccttctatctatctatctatctatctatctgtctgtctgtctgtctgtctgtctatctatctatatctatctatctatcatctatctatccatatctatctatctatctatctatctatcatctatctatctatctatctatctatatctatcgtctatctatccagtctatctacctcctattagtct

SEQ ID NO: 332 - DNA, 20 bp, Artificial Sequence
  synthetic oligonucleotide
caatctgggtgacagagcaa

SEQ ID NO: 333 - DNA, 20 bp, Artificial Sequence
  synthetic oligonucleotide
gccatattcacttgcccact

SEQ ID NO: 334 - DNA, 88 bp, Artificial Sequence
  synthetic oligonucleotide
gacagagcaagaccctgtctcatagatagatagatagatagatagatagatagatagatagatagacagacagacagatagatacatg

SEQ ID NO: 335 - DNA, 84 bp, Artificial Sequence
  synthetic oligonucleotide
gacagagcaagaccctgtctcatagatagatagatagatagatagatagatagatagatagatagacagacagatagatacatg

SEQ ID NO: 336 - DNA, 84 bp, Artificial Sequence; identical to SEQ ID NO: 335
  synthetic oligonucleotide

SEQ ID NO: 337 - DNA, 84 bp, Artificial Sequence; identical to SEQ ID NO: 335
  synthetic oligonucleotide

SEQ ID NO: 338 - DNA, 84 bp, Artificial Sequence; identical to SEQ ID NO: 335
  synthetic oligonucleotide

SEQ ID NO: 339 - DNA, 88 bp, Artificial Sequence; identical to SEQ ID NO: 334
  synthetic oligonucleotide

SEQ ID NO: 340 - DNA, 88 bp, Artificial Sequence; identical to SEQ ID NO: 334
  synthetic oligonucleotide

SEQ ID NO: 341 - DNA, 88 bp, Artificial Sequence
  synthetic oligonucleotide
gacagagcaagaccctgtctcatagatagatagatagatagatagatagatagatagatagatagatagacagacagatagatacatg

SEQ ID NO: 342 - DNA, 88 bp, Artificial Sequence; identical to SEQ ID NO: 341
  synthetic oligonucleotide

SEQ ID NO: 343 - DNA, 88 bp, Artificial Sequence; identical to SEQ ID NO: 341
  synthetic oligonucleotide

SEQ ID NO: 344 - DNA, 88 bp, Artificial Sequence; identical to SEQ ID NO: 341
  synthetic oligonucleotide

SEQ ID NO: 345 - DNA, 88 bp, Artificial Sequence; identical to SEQ ID NO: 341
  synthetic oligonucleotide

SEQ ID NO: 346 - DNA, 88 bp, Artificial Sequence
  synthetic oligonucleotide
gacagagcaagaccctgtctcatagatagatagatagatagatagatagatagatagatagatagatagatagacagatagatacatg

SEQ ID NO: 347 - DNA, 88 bp, Artificial Sequence; identical to SEQ ID NO: 346
  synthetic oligonucleotide

SEQ ID NO: 348 - DNA, 88 bp, Artificial Sequence; identical to SEQ ID NO: 346
  synthetic oligonucleotide

SEQ ID NO: 349 - DNA, 92 bp, Artificial Sequence
  synthetic oligonucleotide
gacagagcaagaccctgtctcatagatagatagatagatagatagatagatagatagatagatagatagacagacagacagatagatacatg

SEQ ID NO: 350 - DNA, 92 bp, Artificial Sequence; identical to SEQ ID NO: 349
  synthetic oligonucleotide

SEQ ID NO: 351 - DNA, 92 bp, Artificial Sequence
  synthetic oligonucleotide
gacagagcaagaccctgtctcatagatagatagatagatagatagatagatagatagatagatagatagatagacagacagatagatacatg

SEQ ID NO: 352 - DNA, 92 bp, Artificial Sequence; identical to SEQ ID NO: 351
  synthetic oligonucleotide

SEQ ID NO: 353 - DNA, 92 bp, Artificial Sequence; identical to SEQ ID NO: 351
  synthetic oligonucleotide

SEQ ID NO: 354 - DNA, 92 bp, Artificial Sequence; identical to SEQ ID NO: 351
  synthetic oligonucleotide

SEQ ID NO: 355 - DNA, 92 bp, Artificial Sequence; identical to SEQ ID NO: 351
  synthetic oligonucleotide

SEQ ID NO: 356 - DNA, 92 bp, Artificial Sequence
  synthetic oligonucleotide
gacagagcaagaccctgtctcatagatagatagatagatagatagatagatagatagatagatagatagatagatagacagatagatacatg

SEQ ID NO: 357 - DNA, 92 bp, Artificial Sequence; identical to SEQ ID NO: 356
  synthetic oligonucleotide

SEQ ID NO: 358 - DNA, 92 bp, Artificial Sequence; identical to SEQ ID NO: 356
  synthetic oligonucleotide

SEQ ID NO: 359 - DNA, 92 bp, Artificial Sequence; identical to SEQ ID NO: 356
  synthetic oligonucleotide

SEQ ID NO: 360 - DNA, 96 bp, Artificial Sequence
  synthetic oligonucleotide
gacagagcaagaccctgtctcatagatagatagatagatagatagatagatagatagatagatagatagatagacagacagacagatagatacatg

SEQ ID NO: 361 - DNA, 96 bp, Artificial Sequence; identical to SEQ ID NO: 360
  synthetic oligonucleotide

SEQ ID NO: 362 - DNA, 96 bp, Artificial Sequence; identical to SEQ ID NO: 360
  synthetic oligonucleotide

SEQ ID NO: 363 - DNA, 96 bp, Artificial Sequence
  synthetic oligonucleotide
gacagagcaagaccctgtctcatagatagatagatagatagatagatagatagatagatagatagatagatagatagacagacagatagatacatg

SEQ ID NO: 364 - DNA, 96 bp, Artificial Sequence; identical to SEQ ID NO: 363
  synthetic oligonucleotide

SEQ ID NO: 365 - DNA, 96 bp, Artificial Sequence; identical to SEQ ID NO: 363
  synthetic oligonucleotide

SEQ ID NO: 366 - DNA, 100 bp, Artificial Sequence
  synthetic oligonucleotide
gacagagcaagaccctgtctcatagatagatagatagatagatagatagatagatagatagatagatagatagatagacagacagacagatagatacatg

SEQ ID NO: 367 - DNA, 100 bp, Artificial Sequence; identical to SEQ ID NO: 366
  synthetic oligonucleotide

SEQ ID NO: 368 - DNA, 100 bp, Artificial Sequence; identical to SEQ ID NO: 366
  synthetic oligonucleotide

SEQ ID NO: 369 - DNA, 100 bp, Artificial Sequence; identical to SEQ ID NO: 366
  synthetic oligonucleotide

SEQ ID NO: 370 - DNA, 100 bp, Artificial Sequence
  synthetic oligonucleotide
gacagagcaagaccctgtctcatagatagatagatagatagatagatagatagatagatagatagatagatagatagatagacagacagatagatacatg

SEQ ID NO: 371 - DNA, 100 bp, Artificial Sequence
  synthetic oligonucleotide
gacagagcaagaccctgtctcatagatagatagatagatagatagatagatagatagatagatagatagatagacagacagacagacagatagatacatg

SEQ ID NO: 372 - DNA, 104 bp, Artificial Sequence
  synthetic oligonucleotide
gacagagcaagaccctgtctcatagatagatagatagatagatagatagatagatagatagatagatagatagatagatagacagacagacagatagatacatg

SEQ ID NO: 373 - DNA, 104 bp, Artificial Sequence; identical to SEQ ID NO: 372
  synthetic oligonucleotide

SEQ ID NO: 374 - DNA, 108 bp, Artificial Sequence
  synthetic oligonucleotide
gacagagcaagaccctgtctcatagatagatagatagatagatagatagatagatagatagatagatagatagatagatagatagacagacagacagatagatacatg

SEQ ID NO: 375 - DNA, 20 bp, Artificial Sequence
  synthetic oligonucleotide
aaaggcagatcccaagctct

SEQ ID NO: 376 - DNA, 20 bp, Artificial Sequence
  synthetic oligonucleotide
gcgtttgtgtgtgcatctgt

SEQ ID NO: 377 - DNA, 61 bp, Artificial Sequence
  synthetic oligonucleotide
gatagatagatagatagatagatagatagatagatagatagatatcattgaaagacaaaac

SEQ ID NO: 378 - DNA, 49 bp, Artificial Sequence
  synthetic oligonucleotide
gatagatagatagatagatagatagatagatatcattgaaagacaaaac

SEQ ID NO: 379 - DNA, 53 bp, Artificial Sequence
  synthetic oligonucleotide
gatagatagatagatagatagatagatagatagatatcattgaaagacaaaac

SEQ ID NO: 380 - DNA, 53 bp, Artificial Sequence; identical to SEQ ID NO: 379
  synthetic oligonucleotide

SEQ ID NO: 381 - DNA, 53 bp, Artificial Sequence; identical to SEQ ID NO: 379
  synthetic oligonucleotide

SEQ ID NO: 382 - DNA, 53 bp, Artificial Sequence
  synthetic oligonucleotide
gatagatagatagatagatagatagatagatagatatcattgaaagacaaacc

SEQ ID NO: 383 - DNA, 53 bp, Artificial Sequence; identical to SEQ ID NO: 382
  synthetic oligonucleotide

SEQ ID NO: 384 - DNA, 53 bp, Artificial Sequence; identical to SEQ ID NO: 382
  synthetic oligonucleotide

SEQ ID NO: 385 - DNA, 53 bp, Artificial Sequence; identical to SEQ ID NO: 382
  synthetic oligonucleotide

SEQ ID NO: 386 - DNA, 57 bp, Artificial Sequence
  synthetic oligonucleotide
gatagatagatagatagatagatagatagatagatagatatcattgaaagacaaaac

SEQ ID NO: 387 - DNA, 57 bp, Artificial Sequence; identical to SEQ ID NO: 386
  synthetic oligonucleotide

SEQ ID NO: 388 - DNA, 57 bp, Artificial Sequence
  synthetic oligonucleotide
gatagatagatagatagatagatagatagatagatagatatcattgaaagacaaacc

SEQ ID NO: 389 - DNA, 57 bp, Artificial Sequence; identical to SEQ ID NO: 388
  synthetic oligonucleotide

SEQ ID NO: 390 - DNA, 61 bp, Artificial Sequence; identical to SEQ ID NO: 377
  synthetic oligonucleotide

SEQ ID NO: 391 - DNA, 61 bp, Artificial Sequence; identical to SEQ ID NO: 377
  synthetic oligonucleotide

SEQ ID NO: 392 - DNA, 61 bp, Artificial Sequence; identical to SEQ ID NO: 377
  synthetic oligonucleotide

SEQ ID NO: 393 - DNA, 61 bp, Artificial Sequence; identical to SEQ ID NO: 377
  synthetic oligonucleotide

SEQ ID NO: 394 - DNA, 65 bp, Artificial Sequence
  synthetic oligonucleotide
gatagatagatagatagatagatagatagatagatagatagatagatatcattgaaagacaaaac

SEQ ID NO: 395 - DNA, 65 bp, Artificial Sequence; identical to SEQ ID NO: 394
  synthetic oligonucleotide

SEQ ID NO: 396 - DNA, 65 bp, Artificial Sequence; identical to SEQ ID NO: 394
  synthetic oligonucleotide

SEQ ID NO: 397 - DNA, 65 bp, Artificial Sequence; identical to SEQ ID NO: 394
  synthetic oligonucleotide

SEQ ID NO: 398 - DNA, 65 bp, Artificial Sequence; identical to SEQ ID NO: 394
  synthetic oligonucleotide

SEQ ID NO: 399 - DNA, 65 bp, Artificial Sequence; identical to SEQ ID NO: 394
  synthetic oligonucleotide

SEQ ID NO: 400 - DNA, 69 bp, Artificial Sequence
  synthetic oligonucleotide
gatagatagatagatagatagatagatagatagatagatagatagatagatatcattgaaagacaaaac

SEQ ID NO: 401 - DNA, 73 bp, Artificial Sequence
  synthetic oligonucleotide
gatagatagatagatagatagatagatagatagatagatagatagatagatagatatcattgaaagacaaaac

SEQ ID NO: 402 - DNA, 73 bp, Artificial Sequence; identical to SEQ ID NO: 401
  synthetic oligonucleotide

SEQ ID NO: 403 - DNA, 20 bp, Artificial Sequence
  synthetic oligonucleotide
cctttgcctgagttttgctc

SEQ ID NO: 404 - DNA, 20 bp, Artificial Sequence
  synthetic oligonucleotide
tatcgtatcccattgcgtga

SEQ ID NO: 405 - DNA, 55 bp, Artificial Sequence
  synthetic oligonucleotide
tatctatctatctatctatctatctatctatctatctatctatctatctattccc

SEQ ID NO: 406 - DNA, 39 bp, Artificial Sequence
  synthetic oligonucleotide
tatctatctatctatctatctatctatctatctattccc

SEQ ID NO: 407 - DNA, 39 bp, Artificial Sequence; identical to SEQ ID NO: 406
  synthetic oligonucleotide

SEQ ID NO: 408 - DNA, 43 bp, Artificial Sequence
  synthetic oligonucleotide
tatctatctatctatctatctatctatctatctatctattccc

SEQ ID NO: 409 - DNA, 43 bp, Artificial Sequence; identical to SEQ ID NO: 408
  synthetic oligonucleotide

SEQ ID NO: 410 - DNA, 47 bp, Artificial Sequence
  synthetic oligonucleotide
tatctatctatctatctatctatctatctatctatctatctattccc

SEQ ID NO: 411 - DNA, 47 bp, Artificial Sequence; identical to SEQ ID NO: 410
  synthetic oligonucleotide

SEQ ID NO: 412 - DNA, 47 bp, Artificial Sequence; identical to SEQ ID NO: 410
  synthetic oligonucleotide

SEQ ID NO: 413 - DNA, 47 bp, Artificial Sequence; identical to SEQ ID NO: 410
  synthetic oligonucleotide

SEQ ID NO: 414 - DNA, 51 bp, Artificial Sequence
  synthetic oligonucleotide
tatctatctatctatctatctatctatctatctatctatctatctattccc

SEQ ID NO: 415 - DNA, 51 bp, Artificial Sequence; identical to SEQ ID NO: 414
  synthetic oligonucleotide

SEQ ID NO: 416 - DNA, 51 bp, Artificial Sequence; identical to SEQ ID NO: 414
  synthetic oligonucleotide

SEQ ID NO: 417 - DNA, 55 bp, Artificial Sequence; identical to SEQ ID NO: 405
  synthetic oligonucleotide

SEQ ID NO: 418 - DNA, 55 bp, Artificial Sequence; identical to SEQ ID NO: 405
  synthetic oligonucleotide

SEQ ID NO: 419 - DNA, 55 bp, Artificial Sequence; identical to SEQ ID NO: 405
  synthetic oligonucleotide

SEQ ID NO: 420 - DNA, 55 bp, Artificial Sequence
  synthetic oligonucleotide
tatctatctgtctatctatctatctatctatctatctatctatctatctattccc

SEQ ID NO: 421 - DNA, 55 bp, Artificial Sequence; identical to SEQ ID NO: 420
  synthetic oligonucleotide

SEQ ID NO: 422 - DNA, 59 bp, Artificial Sequence
  synthetic oligonucleotide
tatctatctatctatctatctatctatctatctatctatctatctatctatctattccc

SEQ ID NO: 423 - DNA, 59 bp, Artificial Sequence; identical to SEQ ID NO: 422
  synthetic oligonucleotide

SEQ ID NO: 424 - DNA, 59 bp, Artificial Sequence
  synthetic oligonucleotide
tatctatctgtctatctatctatctatctatctatctatctatctatctatctattccc

SEQ ID NO: 425 - DNA, 59 bp, Artificial Sequence; identical to SEQ ID NO: 424
  synthetic oligonucleotide

SEQ ID NO: 426 - DNA, 59 bp, Artificial Sequence; identical to SEQ ID NO: 424
  synthetic oligonucleotide

SEQ ID NO: 427 - DNA, 59 bp, Artificial Sequence; identical to SEQ ID NO: 424
  synthetic oligonucleotide

SEQ ID NO: 428 - DNA, 59 bp, Artificial Sequence
  synthetic oligonucleotide
tatctatctgtctgtctatctatctatctatctatctatctatctatctatctattccc

SEQ ID NO: 429 - DNA, 63 bp, Artificial Sequence
  synthetic oligonucleotide
tatctatctatctatctatctatctatctatctatctatctatctatctatctatctattccc

SEQ ID NO: 430 - DNA, 63 bp, Artificial Sequence; identical to SEQ ID NO: 429
  synthetic oligonucleotide

SEQ ID NO: 431 - DNA, 63 bp, Artificial Sequence
  synthetic oligonucleotide
tatctatctgtctatctatctatctatctatctatctatctatctatctatctatctattccc

SEQ ID NO: 432 - DNA, 63 bp, Artificial Sequence; identical to SEQ ID NO: 431
  synthetic oligonucleotide

SEQ ID NO: 433 - DNA, 63 bp, Artificial Sequence
  synthetic oligonucleotide
tatctatctatctgtctatctatctatctatctatctatctatctatctatctatctattccc

SEQ ID NO: 434 - DNA, 63 bp, Artificial Sequence
  synthetic oligonucleotide
tatctatctgtctgtctatctatctatctatctatctatctatctatctatctatctattccc

SEQ ID NO: 435 - DNA, 63 bp, Artificial Sequence
  synthetic oligonucleotide
tatctatctgtgtatctatctatctatctatctatctatctatctatctatctatctattccc

SEQ ID NO: 436 - DNA, 67 bp, Artificial Sequence
  synthetic oligonucleotide
tatctatctatctgtctatctatctatctatctatctatctatctatctatctatctatctattccc

SEQ ID NO: 437 - DNA, 67 bp, Artificial Sequence; identical to SEQ ID NO: 436
  synthetic oligonucleotide

SEQ ID NO: 438 - DNA, 67 bp, Artificial Sequence; identical to SEQ ID NO: 436
  synthetic oligonucleotide

SEQ ID NO: 439 - DNA, 67 bp, Artificial Sequence
  synthetic oligonucleotide
tatctatctgtctatctatctatctatctatctatctatctatctatctatctatctatctattccc

SEQ ID NO: 440 - DNA, 71 bp, Artificial Sequence
  synthetic oligonucleotide
tatctatctatctgtctatctatctatctatctatctatctatctatctatctatctatctatctattccc

SEQ ID NO: 441 - DNA, 71 bp, Artificial Sequence; identical to SEQ ID NO: 440
  synthetic oligonucleotide

SEQ ID NO: 442 - DNA, 20 bp, Artificial Sequence
  synthetic oligonucleotide
ttagagacggggtttcacca

SEQ ID NO: 443 - DNA, 21 bp, Artificial Sequence
  synthetic oligonucleotide
tcattgacagaattgcaccaa

SEQ ID NO: 444 - DNA, 121 bp, Artificial Sequence
  synthetic oligonucleotide
taacgatagatagatagatagatagatagatagatagatagatagatagatagatagacagattgatagtttttttttatctcactaaatagtctatagtaaacatttaattaccaatatt

SEQ ID NO: 445 - DNA, 97 bp, Artificial Sequence
  synthetic oligonucleotide
taacgatagatagatagatagatagatagatagacagattgatagtttttttttatctcactaaatagtctatagtaaacatttaattaccaatatt

SEQ ID NO: 446 - DNA, 97 bp, Artificial Sequence; identical to SEQ ID NO: 445
  synthetic oligonucleotide

SEQ ID NO: 447 - DNA, 101 bp, Artificial Sequence
  synthetic oligonucleotide
taacgatagatagatagatagatagatagatagatagacagattgatagtttttttttatctcactaaatagtctatagtaaacatttaattaccaatatt

SEQ ID NO: 448 - DNA, 101 bp, Artificial Sequence; identical to SEQ ID NO: 447
  synthetic oligonucleotide

SEQ ID NO: 449 - DNA, 101 bp, Artificial Sequence; identical to SEQ ID NO: 447
  synthetic oligonucleotide

SEQ ID NO: 450 - DNA, 101 bp, Artificial Sequence; identical to SEQ ID NO: 447
  synthetic oligonucleotide

SEQ ID NO: 451 - DNA, 101 bp, Artificial Sequence; identical to SEQ ID NO: 447
  synthetic oligonucleotide

SEQ ID NO: 452 - DNA, 105 bp, Artificial Sequence
  synthetic oligonucleotide
taacgatagatagatagatagatagatagatagatagatagacagattgatagtttttttttatctcactaaatagtctatagtaaacatttaattaccaatatt

SEQ ID NO: 453 - DNA, 105 bp, Artificial Sequence; identical to SEQ ID NO: 452
  synthetic oligonucleotide

SEQ ID NO: 454 - DNA, 105 bp, Artificial Sequence; identical to SEQ ID NO: 452
  synthetic oligonucleotide

SEQ ID NO: 455 - DNA, 109 bp, Artificial Sequence
  synthetic oligonucleotide
taacgatagatagatagatagatagatagatagatagatagatagacagattgatagtttttttttatctcactaaatagtctatagtaaacatttaattaccaatatt

SEQ ID NO: 456 - DNA, 109 bp, Artificial Sequence; identical to SEQ ID NO: 455
  synthetic oligonucleotide

SEQ ID NO: 457 - DNA, 109 bp, Artificial Sequence; identical to SEQ ID NO: 455
  synthetic oligonucleotide

SEQ ID NO: 458 - DNA, 109 bp, Artificial Sequence; identical to SEQ ID NO: 455
  synthetic oligonucleotide

SEQ ID NO: 459 - DNA, 109 bp, Artificial Sequence
  synthetic oligonucleotide
taacgatagatagatagatagatagatagatagatagatagatagacagattgatagttttttttaatctcactaaatagtctatagtaaacatttaattaccaatatt

SEQ ID NO: 460 - DNA, 109 bp, Artificial Sequence; identical to SEQ ID NO: 459
  synthetic oligonucleotide

SEQ ID NO: 461 - DNA, 109 bp, Artificial Sequence; identical to SEQ ID NO: 459
  synthetic oligonucleotide

SEQ ID NO: 462 - DNA, 109 bp, Artificial Sequence
  synthetic oligonucleotide
taacgatagatagatagatagatagatagatagatagatagatagacagattgatagtttttttttatctcactaaatagtctatagtaaacatttaattaccaatatg

SEQ ID NO: 463 - DNA, 113 bp, Artificial Sequence
  synthetic oligonucleotide
taacgatagatagatagatagatagatagatagatagatagatagatagacagattgatagtttttttttatctcactaaatagtctatagtaaacatttaattaccaatatt

SEQ ID NO: 464 - DNA, 113 bp, Artificial Sequence; identical to SEQ ID NO: 463
  synthetic oligonucleotide

SEQ ID NO: 465 - DNA, 113 bp, Artificial Sequence; identical to SEQ ID NO: 463
  synthetic oligonucleotide

SEQ ID NO: 466 - DNA, 113 bp, Artificial Sequence; identical to SEQ ID NO: 463
  synthetic oligonucleotide

SEQ ID NO: 467 - DNA, 113 bp, Artificial Sequence
  synthetic oligonucleotide
taacgatagatagatagatagatagatagatagatagatagatagatagacagattgatagttttttttaatctcactaaatagtctatagtaaacatttaattaccaatatt

SEQ ID NO: 468 - DNA, 113 bp, Artificial Sequence; identical to SEQ ID NO: 467
  synthetic oligonucleotide

SEQ ID NO: 469 - DNA, 113 bp, Artificial Sequence; identical to SEQ ID NO: 467
  synthetic oligonucleotide

SEQ ID NO: 470 - DNA, 113 bp, Artificial Sequence; identical to SEQ ID NO: 467
  synthetic oligonucleotide

SEQ ID NO: 471 - DNA, 113 bp, Artificial Sequence
  synthetic oligonucleotide
taacgatagatagatagatagatagatagatagatagatagatagatagacagattgatagtttttttttatctcactaaatagtctatagtaaacatttaattaccaatatg

SEQ ID NO: 472 - DNA, 113 bp, Artificial Sequence; identical to SEQ ID NO: 471
  synthetic oligonucleotide

SEQ ID NO: 473 - DNA, 117 bp, Artificial Sequence
  synthetic oligonucleotide
taacgatagatagatagatagatagatagatagatagatagatagatagatagacagattgatagtttttttttatctcactaaatagtctatagtaaacatttaattaccaatatt

SEQ ID NO: 474 - DNA, 117 bp, Artificial Sequence; identical to SEQ ID NO: 473
  synthetic oligonucleotide

SEQ ID NO: 475 - DNA, 117 bp, Artificial Sequence; identical to SEQ ID NO: 473
  synthetic oligonucleotide

SEQ ID NO: 476 - DNA, 117 bp, Artificial Sequence; identical to SEQ ID NO: 473
  synthetic oligonucleotide

SEQ ID NO: 477 - DNA, 117 bp, Artificial Sequence
  synthetic oligonucleotide
taacgatagatagatagatagatagatagatagatagatagatagatagatagacagattgatagttttttttaatctcactaaatagtctatagtaaacatttaattaccaatatt

SEQ ID NO: 478 - DNA, 117 bp, Artificial Sequence; identical to SEQ ID NO: 477
  synthetic oligonucleotide

SEQ ID NO: 479 - DNA, 117 bp, Artificial Sequence; identical to SEQ ID NO: 477
  synthetic oligonucleotide

SEQ ID NO: 480 - DNA, 121 bp, Artificial Sequence; identical to SEQ ID NO: 444
  synthetic oligonucleotide

SEQ ID NO: 481 - DNA, 121 bp, Artificial Sequence; identical to SEQ ID NO: 444
  synthetic oligonucleotide

SEQ ID NO: 482 - DNA, 20 bp, Artificial Sequence
  synthetic oligonucleotide
gaagtctgggatgtggagga

SEQ ID NO: 483 - DNA, 20 bp, Artificial Sequence
  synthetic oligonucleotide
tccttcaacttgggttgagc

SEQ ID NO: 484 - DNA, 65 bp, Artificial Sequence
  synthetic oligonucleotide
tctgtatttacaaatacattatctatctatctatctatctatctatctatctatctatctatcaa

SEQ ID NO: 485 - DNA, 53 bp, Artificial Sequence
  synthetic oligonucleotide
tctgtatttacaaatacattatctatctatctatctatctatctatctatcaa

SEQ ID NO: 486 - DNA, 53 bp, Artificial Sequence; identical to SEQ ID NO: 485
  synthetic oligonucleotide

SEQ ID NO: 487 - DNA, 53 bp, Artificial Sequence; identical to SEQ ID NO: 485
  synthetic oligonucleotide

SEQ ID NO: 488 - DNA, 57 bp, Artificial Sequence
  synthetic oligonucleotide
tctgtatttacaaatacattatctatctatctatctatctatctatctatctatcaa

SEQ ID NO: 489 - DNA, 57 bp, Artificial Sequence; identical to SEQ ID NO: 488
  synthetic oligonucleotide

SEQ ID NO: 490 - DNA, 57 bp, Artificial Sequence; identical to SEQ ID NO: 488
  synthetic oligonucleotide

SEQ ID NO: 491 - DNA, 61 bp, Artificial Sequence
  synthetic oligonucleotide
tctgtatttacaaatacattatctatctatctatctatctatctatctatctatctatcaa

SEQ ID NO: 492 - DNA, 61 bp, Artificial Sequence; identical to SEQ ID NO: 491
  synthetic oligonucleotide

SEQ ID NO: 493 - DNA, 61 bp, Artificial Sequence
  synthetic oligonucleotide
tctgtatttacaaatacattatctatctatctatctatctatctatctatctatctatcta

SEQ ID NO: 494 - DNA, 61 bp, Artificial Sequence; identical to SEQ ID NO: 493
  synthetic oligonucleotide

SEQ ID NO: 495 - DNA, 65 bp, Artificial Sequence; identical to SEQ ID NO: 484
  synthetic oligonucleotide

SEQ ID NO: 496 - DNA, 65 bp, Artificial Sequence; identical to SEQ ID NO: 484
  synthetic oligonucleotide

SEQ ID NO: 497 - DNA, 65 bp, Artificial Sequence; identical to SEQ ID NO: 484
  synthetic oligonucleotide

SEQ ID NO: 498 - DNA, 65 bp, Artificial Sequence; identical to SEQ ID NO: 484
  synthetic oligonucleotide

SEQ ID NO: 499 - DNA, 65 bp, Artificial Sequence
  synthetic oligonucleotide
tctgtatttacaaatacattatctatctatctatctatctatctatctatctatctatctatcta

SEQ ID NO: 500 - DNA, 65 bp, Artificial Sequence; identical to SEQ ID NO: 499
  synthetic oligonucleotide

SEQ ID NO: 501 - DNA, 65 bp, Artificial Sequence; identical to SEQ ID NO: 499
  synthetic oligonucleotide

SEQ ID NO: 502 - DNA, 65 bp, Artificial Sequence; identical to SEQ ID NO: 499
  synthetic oligonucleotide

SEQ ID NO: 503 - DNA, 69 bp, Artificial Sequence
  synthetic oligonucleotide
tctgtatttacaaatacattatctatctatctatctatctatctatctatctatctatctatctatcaa

SEQ ID NO: 504 - DNA, 69 bp, Artificial Sequence; identical to SEQ ID NO: 503
  synthetic oligonucleotide

SEQ ID NO: 505 - DNA, 69 bp, Artificial Sequence; identical to SEQ ID NO: 503
  synthetic oligonucleotide

SEQ ID NO: 506 - DNA, 69 bp, Artificial Sequence
  synthetic oligonucleotide
tctgtatttacaaatacattatctatctatctatctatctatctatctatctatctatctatctatcta

SEQ ID NO: 507 - DNA, 69 bp, Artificial Sequence; identical to SEQ ID NO: 506
  synthetic oligonucleotide

SEQ ID NO: 508 - DNA, 68 bp, Artificial Sequence
  synthetic oligonucleotide
ctgtatttacaaatacattatctatctatctatctatctatctatctatctatctatctatctatcta

SEQ ID NO: 509 - DNA, 72 bp, Artificial Sequence
  synthetic oligonucleotide
ctgtatttacaaatacattatctatctatctatctatctatctatctatctatctatctatctatctatcaa

SEQ ID NO: 510 - DNA, 73 bp, Artificial Sequence
  synthetic oligonucleotide
tctgtatttacaaatacattatctatctatctatctatctatctatctatctatctatctatctatctatcaa

SEQ ID NO: 511 - DNA, 73 bp, Artificial Sequence
  synthetic oligonucleotide
tctgtatttacaaatacattatctatctatctatctatctatctatctatctatctatctatctatctatcta

SEQ ID NO: 512 - DNA, 73 bp, Artificial Sequence; identical to SEQ ID NO: 511
  synthetic oligonucleotide

SEQ ID NO: 513 - DNA, 73 bp, Artificial Sequence; identical to SEQ ID NO: 511
  synthetic oligonucleotide

SEQ ID NO: 514 - DNA, 77 bp, Artificial Sequence
  synthetic oligonucleotide
tctgtatttacaaatacattatctatctatctatctatctatctatctatctatctatctatctatctatctatcaa

SEQ ID NO: 515 - DNA, 77 bp, Artificial Sequence; identical to SEQ ID NO: 514
  synthetic oligonucleotide

SEQ ID NO: 516 - DNA, 77 bp, Artificial Sequence
  synthetic oligonucleotide
tctgtatttacaaatacattatctatctatctatctatctatctatctatctatctatctatctatctatctatcta

SEQ ID NO: 517 - DNA, 76 bp, Artificial Sequence
  synthetic oligonucleotide
ctgtatttacaaatacattatctatctatctatctatctatctatctatctatctatctatctatctatctatcta

SEQ ID NO: 518 - DNA, 22 bp, Artificial Sequence
  synthetic oligonucleotide
agcaagtatgtgacaagggtga

SEQ ID NO: 519 - DNA, 20 bp, Artificial Sequence
  synthetic oligonucleotide
gattccaatcatagccacag

SEQ ID NO: 520 - DNA, 70 bp, Artificial Sequence
  synthetic oligonucleotide
atccttatgtaatattttgaagatagatagatagatagatagatagatagatagatagatagatagaggt

SEQ ID NO: 521 - DNA, 62 bp, Artificial Sequence
  synthetic oligonucleotide
atccttacgtaatattttgaagatagatagatagatagatagatagatagatagatagatgt

SEQ ID NO: 522 - DNA, 62 bp, Artificial Sequence; identical to SEQ ID NO: 521
  synthetic oligonucleotide

SEQ ID NO: 523 - DNA, 62 bp, Artificial Sequence; identical to SEQ ID NO: 521
  synthetic oligonucleotide

SEQ ID NO: 524 - DNA, 66 bp, Artificial Sequence
  synthetic oligonucleotide
atccttatgtaatattttgaagatagatagatagatagatagatagatagatagatagatagaggt

SEQ ID NO: 525 - DNA, 66 bp, Artificial Sequence; identical to SEQ ID NO: 524
  synthetic oligonucleotide

SEQ ID NO: 526 - DNA, 66 bp, Artificial Sequence; identical to SEQ ID NO: 524
  synthetic oligonucleotide

SEQ ID NO: 527 - DNA, 66 bp, Artificial Sequence
  synthetic oligonucleotide
atccttacgtaatattttgaagatagatagatagatagatagatagatagatagatagatagaggt

SEQ ID NO: 528 - DNA, 66 bp, Artificial Sequence; identical to SEQ ID NO: 527
  synthetic oligonucleotide

SEQ ID NO: 529 - DNA, 66 bp, Artificial Sequence; identical to SEQ ID NO: 527
  synthetic oligonucleotide

SEQ ID NO: 530 - DNA, 66 bp, Artificial Sequence
  synthetic oligonucleotide
atccttacgtaatattttgaagatagatagatagatagatagatagatagatagatagatagatgt

SEQ ID NO: 531 - DNA, 66 bp, Artificial Sequence; identical to SEQ ID NO: 530
  synthetic oligonucleotide

SEQ ID NO: 532 - DNA, 66 bp, Artificial Sequence; identical to SEQ ID NO: 530
  synthetic oligonucleotide

SEQ ID NO: 533 - DNA, 70 bp, Artificial Sequence; identical to SEQ ID NO: 520
  synthetic oligonucleotide

SEQ ID NO: 534 - DNA, 70 bp, Artificial Sequence; identical to SEQ ID NO: 520
  synthetic oligonucleotide

SEQ ID NO: 535 - DNA, 70 bp, Artificial Sequence; identical to SEQ ID NO: 520
  synthetic oligonucleotide

SEQ ID NO: 536 - DNA, 70 bp, Artificial Sequence
  synthetic oligonucleotide
atccttacgtaatattttgaagatagatagatagatagatagatagatagatagatagatagatagaggt

SEQ ID NO: 537 - DNA, 70 bp, Artificial Sequence; identical to SEQ ID NO: 536
  synthetic oligonucleotide

SEQ ID NO: 538 - DNA, 70 bp, Artificial Sequence; identical to SEQ ID NO: 536
  synthetic oligonucleotide

SEQ ID NO: 539 - DNA, 70 bp, Artificial Sequence; identical to SEQ ID NO: 536
  synthetic oligonucleotide

SEQ ID NO: 540 - DNA, 70 bp, Artificial Sequence
  synthetic oligonucleotide
atccttacgtaatattttgaagatagatagatagatagatagatagatagatagatagatagatagatgt

SEQ ID NO: 541 - DNA, 70 bp, Artificial Sequence; identical to SEQ ID NO: 540
  synthetic oligonucleotide

SEQ ID NO: 542 - DNA, 70 bp, Artificial Sequence; identical to SEQ ID NO: 540
  synthetic oligonucleotide

SEQ ID NO: 543 - DNA, 74 bp, Artificial Sequence
  synthetic oligonucleotide
atccttatgtaatattttgaagatagatagatagatagatagatagatagatagatagatagatagatagaggt

SEQ ID NO: 544 - DNA, 74 bp, Artificial Sequence; identical to SEQ ID NO: 543
  synthetic oligonucleotide

SEQ ID NO: 545 - DNA, 74 bp, Artificial Sequence; identical to SEQ ID NO: 543
  synthetic oligonucleotide

SEQ ID NO: 546 - DNA, 74 bp, Artificial Sequence
  synthetic oligonucleotide
atccttacgtaatattttgaagatagatagatagatagatagatagatagatagatagatagatagatagaggt

SEQ ID NO: 547 - DNA, 74 bp, Artificial Sequence; identical to SEQ ID NO: 546
  synthetic oligonucleotide

SEQ ID NO: 548 - DNA, 74 bp, Artificial Sequence; identical to SEQ ID NO: 546
  synthetic oligonucleotide

SEQ ID NO: 549 - DNA, 74 bp, Artificial Sequence; identical to SEQ ID NO: 546
  synthetic oligonucleotide

SEQ ID NO: 550 - DNA, 74 bp, Artificial Sequence
  synthetic oligonucleotide
atccttacgtaatattttgaagatagatagatagatagatagatagatagatagatagatagatagatagatgt

SEQ ID NO: 551 - DNA, 74 bp, Artificial Sequence; identical to SEQ ID NO: 550
  synthetic oligonucleotide

SEQ ID NO: 552 - DNA, 74 bp, Artificial Sequence; identical to SEQ ID NO: 550
  synthetic oligonucleotide

SEQ ID NO: 553 - DNA, 74 bp, Artificial Sequence; identical to SEQ ID NO: 550
  synthetic oligonucleotide

SEQ ID NO: 554 - DNA, 74 bp, Artificial Sequence; identical to SEQ ID NO: 550
  synthetic oligonucleotide

SEQ ID NO: 555 - DNA, 78 bp, Artificial Sequence
  synthetic oligonucleotide
atccttatgtaatattttgaagatagatagatagatagatagatagatagatagatagatagatagatagatagaggt

SEQ ID NO: 556 - DNA, 78 bp, Artificial Sequence; identical to SEQ ID NO: 555
  synthetic oligonucleotide

SEQ ID NO: 557 - DNA, 78 bp, Artificial Sequence
  synthetic oligonucleotide
atccttacgtaatattttgaagatagatagatagatagatagatagatagatagatagatagatagatagatagaggt

SEQ ID NO: 558 - DNA, 78 bp, Artificial Sequence; identical to SEQ ID NO: 557
  synthetic oligonucleotide

SEQ ID NO: 559 - DNA, 78 bp, Artificial Sequence; identical to SEQ ID NO: 557
  synthetic oligonucleotide

SEQ ID NO: 560 - DNA, 78 bp, Artificial Sequence
  synthetic oligonucleotide
atccttacgtaatattttgaagatagatagatagatagatagatagatagatagatagatagatagatagatagatgt

SEQ ID NO: 561 - DNA, 78 bp, Artificial Sequence; identical to SEQ ID NO: 560
  synthetic oligonucleotide

SEQ ID NO: 562 - DNA, 78 bp, Artificial Sequence; identical to SEQ ID NO: 560
  synthetic oligonucleotide

SEQ ID NO: 563 - DNA, 82 bp, Artificial Sequence
  synthetic oligonucleotide
atccttatgtaatattttgaagatagatagatagatagatagatagatagatagatagatagatagatagatagatagaggt

SEQ ID NO: 564 - DNA, 82 bp, Artificial Sequence; identical to SEQ ID NO: 563
  synthetic oligonucleotide

SEQ ID NO: 565 - DNA, 82 bp, Artificial Sequence
  synthetic oligonucleotide
atccttacgtaatattttgaagatagatagatagatagatagatagatagatagatagatagatagatagatagatagatgt

SEQ ID NO: 566 - DNA, 82 bp, Artificial Sequence; identical to SEQ ID NO: 565
  synthetic oligonucleotide

SEQ ID NO: 567 - DNA, 20 bp, Artificial Sequence
  synthetic oligonucleotide
ccctaatgcacccaacattc

SEQ ID NO: 568 - DNA, 20 bp, Artificial Sequence
  synthetic oligonucleotide
tggagctaagtggctgtggt

SEQ ID NO: 569 - DNA, 48 bp, Artificial Sequence
  synthetic oligonucleotide
ctatctatctatctatctatctatctatctatctatctatctatctat

SEQ ID NO: 570 - DNA, 40 bp, Artificial Sequence
  synthetic oligonucleotide
ctatctatctatctatctatctatctatctatctatctat

SEQ ID NO: 571 - DNA, 40 bp, Artificial Sequence; identical to SEQ ID NO: 570
  synthetic oligonucleotide

SEQ ID NO: 572 - DNA, 40 bp, Artificial Sequence; identical to SEQ ID NO: 570
  synthetic oligonucleotide

SEQ ID NO: 573 - DNA, 40 bp, Artificial Sequence; identical to SEQ ID NO: 570
  synthetic oligonucleotide

SEQ ID NO: 574 - DNA, 40 bp, Artificial Sequence; identical to SEQ ID NO: 570
  synthetic oligonucleotide

SEQ ID NO: 575 - DNA, 40 bp, Artificial Sequence; identical to SEQ ID NO: 570
  synthetic oligonucleotide

SEQ ID NO: 576 - DNA, 40 bp, Artificial Sequence
  synthetic oligonucleotide
ctatctatctatctatctatctatctatctatctgtctat

SEQ ID NO: 577 - DNA, 40 bp, Artificial Sequence; identical to SEQ ID NO: 576
  synthetic oligonucleotide

SEQ ID NO: 578 - DNA, 44 bp, Artificial Sequence
  synthetic oligonucleotide
ctatctatctatctatctatctatctatctatctatctatctat

SEQ ID NO: 579 - DNA, 44 bp, Artificial Sequence; identical to SEQ ID NO: 578
  synthetic oligonucleotide

SEQ ID NO: 580 - DNA, 44 bp, Artificial Sequence; identical to SEQ ID NO: 578
  synthetic oligonucleotide

SEQ ID NO: 581 - DNA, 44 bp, Artificial Sequence; identical to SEQ ID NO: 578
  synthetic oligonucleotide

SEQ ID NO: 582 - DNA, 44 bp, Artificial Sequence; identical to SEQ ID NO: 578
  synthetic oligonucleotide

SEQ ID NO: 583 - DNA, 44 bp, Artificial Sequence; identical to SEQ ID NO: 578
  synthetic oligonucleotide

SEQ ID NO: 584 - DNA, 44 bp, Artificial Sequence; identical to SEQ ID NO: 578
  synthetic oligonucleotide

SEQ ID NO: 585 - DNA, 44 bp, Artificial Sequence; identical to SEQ ID NO: 578
  synthetic oligonucleotide

SEQ ID NO: 586 - DNA, 44 bp, Artificial Sequence; identical to SEQ ID NO: 578
  synthetic oligonucleotide

SEQ ID NO: 587 - DNA, 44 bp, Artificial Sequence
  synthetic oligonucleotide
ctatctatctatctatctatctatctatctatctatctgtctat

SEQ ID NO: 588 - DNA, 44 bp, Artificial Sequence; identical to SEQ ID NO: 587
  synthetic oligonucleotide

SEQ ID NO: 589 - DNA, 47 bp, Artificial Sequence
  synthetic oligonucleotide
ctatctatctatctatcatctatctatctatctatctatctatctat

SEQ ID NO: 590 - DNA, 47 bp, Artificial Sequence; identical to SEQ ID NO: 589
  synthetic oligonucleotide

SEQ ID NO: 591 - DNA, 47 bp, Artificial Sequence; identical to SEQ ID NO: 589
  synthetic oligonucleotide

SEQ ID NO: 592 - DNA, 47 bp, Artificial Sequence; identical to SEQ ID NO: 589
  synthetic oligonucleotide

SEQ ID NO: 593 - DNA, 48 bp, Artificial Sequence; identical to SEQ ID NO: 569
  synthetic oligonucleotide

SEQ ID NO: 594 - DNA, 48 bp, Artificial Sequence; identical to SEQ ID NO: 569
  synthetic oligonucleotide

SEQ ID NO: 595 - DNA, 48 bp, Artificial Sequence; identical to SEQ ID NO: 569
  synthetic oligonucleotide

SEQ ID NO: 596 - DNA, 48 bp, Artificial Sequence
  synthetic oligonucleotide
ctatctatctatctatctatctatctatctatctatttatctatctat

SEQ ID NO: 597 - DNA, 52 bp, Artificial Sequence
  synthetic oligonucleotide
ctatctatctatctatctatctatctatctatctatctatctatctatctat

SEQ ID NO: 598 - DNA, 52 bp, Artificial Sequence; identical to SEQ ID NO: 597
  synthetic oligonucleotide

SEQ ID NO: 599 - DNA, 52 bp, Artificial Sequence
  synthetic oligonucleotide
ctatctatctatctatctatctatctatctatctatctatttatctatctat

SEQ ID NO: 600 - DNA, 52 bp, Artificial Sequence; identical to SEQ ID NO: 599
  synthetic oligonucleotide

SEQ ID NO: 601 - DNA, 56 bp, Artificial Sequence
  synthetic oligonucleotide
ctatctatctatctatctatctatctatctatctatctatctatttatctatctat

SEQ ID NO: 602 - DNA, 56 bp, Artificial Sequence; identical to SEQ ID NO: 601
  synthetic oligonucleotide

SEQ ID NO: 603 - DNA, 56 bp, Artificial Sequence; identical to SEQ ID NO: 601
  synthetic oligonucleotide

SEQ ID NO: 604 - DNA, 56 bp, Artificial Sequence; identical to SEQ ID NO: 601
  synthetic oligonucleotide

SEQ ID NO: 605 - DNA, 60 bp, Artificial Sequence
  synthetic oligonucleotide
ctatctatctatctatctatctatctatctatctatctatctatctatttatctatctat

SEQ ID NO: 606 - DNA, 60 bp, Artificial Sequence; identical to SEQ ID NO: 605
  synthetic oligonucleotide

SEQ ID NO: 607 - DNA, 60 bp, Artificial Sequence; identical to SEQ ID NO: 605
  synthetic oligonucleotide

(19) United States
(12) Patent Application Publication
(10) Pub. No.: US 2009/0258354 A1
(43) Pub. Date: Oct. 15, 2009

(54) Methods for DNA Length and Sequence Determination

(76) Inventors: Herbert Oberacher, (US); Walther Parson, Innsbruck (AT)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/249,825

(22) Filed: Oct. 10, 2008

Related U.S. Application Data
(60) Provisional application No. 60/979,360, filed on Oct. 11, 2007.

(51) Int. Cl. C12Q 1/68

(52) U.S. Cl. 435/6

(57) ABSTRACT

Methods for determining nucleic acid length and sequence variation are provided, for example, between an unknown sample and a reference sample. In various embodiments, a method amplifies one or more specific regions of an oligonucleotide molecule in a sample comprising oligonucleotide molecules to produce a sample of amplicons, the sample of amplicons comprising two or more different amplicons. In various embodiments, two or more specific regions are amplified. In various embodiments, the methods (i) denature the amplicons in the sample of amplicons to produce a first set of single-stranded amplicons and a second set of single-stranded amplicons, single stranded amplicons of the second set being complementary to the corresponding single stranded amplicons of the first set and (ii) subject at least the first and second set of single stranded amplicons to mass spectrometric analysis to obtain the masses of the amplicons in the first and second set of single-stranded amplicons. The masses of the amplicons are then used, at least ultimately in part, to determine nucleic acid length and sequence variation.

### CROSS-REFERENCE TO RELATED APPLICATIONS

Pursuant to 35 U.S.C. § 119 (e), this application claims priority to the filing dates of: U.S. Provisional Patent Application Ser. No. 60/979,360 filed on Oct. 11, 2007, the disclosure of which application is herein incorporated by reference.

### INTRODUCTION

DNA typing is one of the most powerful methods for determining the origin of biological traces in forensic casework (M. A. Jobling et al. (2004) Nat. Rev. Genet. 5: 739-751). National DNA databases that have been established as intelligence tool through-out the past decade now contain millions of “genetic fingerprints” that effectively help to link an unknown stain to the true perpetrator. The “genetic fingerprint” that contains the evidential information consists of the combined genotyping information obtained from a selected number of short tandem repeat (STR) loci (J. M. Butler (2006) Forensic Sci. 51: 253-265). STRs are DNA segments typically found in noncoding regions of the human genome and are composed of repeating units of di- to hexanucleotide sequence motifs. The elevated mutation rate of STRs has led to a high degree of polymorphism in humans, which renders STR-typing useful for identity testing. Harmonization of technology and of STR-markers has led to the selection of core loci by the forensic community and constitute the basic configuration of national DNA databases. The International Standard Set of Loci (ISSOL) that is recommended by the Interpol DNA Monitoring Expert Group (www.interpol.int/Public/Forensie/DNA/DNAMEG.asp) involves the STR-loci vWA, TH01, D21 S11, FGA, D8S1179, D18S51 and D3S1358. Depending on the typing chemistry that is used by the laboratory the following STR-loci add to the standard set: D2S1338, D19S433, D16S539, D7S820, D13S317, D5S818, CSF I PO, Penta D, Penta E, TPDX, and SE33. In a recent attempt to identify samples of degraded DNA, so-called “mini-STRs”, D2S44I, DI 0S124, D22S1045, have been evaluated and suggested as additional loci (P. Gill et al. (2006) Forensic Sci. Int. 163: 155-157).

STR-typing is traditionally accomplished via selective amplification using the polymerase chain reaction (PCR) and consecutive electrophoretic analysis (J. M. Butler et al. (2004) Electrophoresis 25: 1397-1412). The PCR amplicons typically range between 100 and 400 base pairs (bp). Their fragment length is determined via the comparison of observed migration times to those of size standards. The individual alleles are denoted by comparing their migration times to those of the allelic ladder, a selection of sequenced allele variants that need to be co-analyzed with the samples in question. So far, capillary electrophoresis (CE) with multi-color fluorescence detection represents the method of choice for STR typing, as it can offer 1-bp-resolution for the discrimination of all allelic length variants within an STR-fingerprint.

### SUMMARY

In various aspects, the present teaching provides methods for determining nucleic acid length and sequence variation, for example, between an unknown sample and a reference sample. In various embodiments, a method amplifies one or more specific regions of an oligonucleotide molecule in a sample comprising oligonucleotide molecules to produce a sample of amplicons, the sample of amplicons comprising two or more different amplicons. In various embodiments, two or more specific regions are amplified. In various embodiments, the methods (i) denature the amplicons in the sample of amplicons to produce a first set of single-stranded amplicons and a second set of single-stranded amplicons, single stranded amplicons of the second set being complementary to the corresponding single stranded amplicons of the first set and (ii) subject at least the first and second set of single stranded amplicons to mass spectrometric analysis to obtain the masses of the amplicons in the first and second set of single-stranded amplicons. The masses of the amplicons are then used, at least ultimately in part, to determine nucleic acid length and sequence variation.

In various embodiments, the step of amplifying one or more specific regions of an oligonucleotide molecule in a sample uses a multiplex-PCR approach to generate amplicons in a multiplex fashion.

In comparison to traditional direct sequencing methods which rely upon fragmentation to determine sequence information, various aspects the present teachings measure the masses of intact amplicons without the need for fragmentation, for example, the masses of the amplicons in the first and second set of single-stranded amplicons. In various embodiments, these measured molecular masses are compared to the mass(es) expected and/or calculated for one or more reference nucleic acid sequences. In various embodiments, the composition of two sequences is considered substantially identical if the molecular mass difference is smaller than the typically observed mass measurement error. In various embodiments, molecular mass differences exceeding the typically observed measurement error indicate the presence of a sequence variation (variation either in length or nucleotide composition).

In various embodiments, the kind of sequence variation can be deduced from the magnitude of the observed mass difference (see., e.g., Table 1). In various embodiments, a sequence variation detected in the first set of single-stranded amplicons is compared to the sequence variation detected in the second set of single-stranded aniplicons complimentary to the first, to determine and or confirm the sequence variation based on the sequence variation in the first set being substantially complimentary to the sequence variation in the second set.

The foregoing and other aspects, embodiments, and features of the teachings can be more fully understood from the following description in conjunction with the accompanying drawings. In the drawings like reference characters generally refer to like features and structural elements throughout the various figures. The drawings are not necessarily to scale, emphasis instead being placed upon illustrating the principles of the teachings.

### BRIEF DESCRIPTION OF THE DRAWINGS

FIGS. 1A-B present data on ICEMS results obtained from two different PCR amplifications of a sample harboring the alleles 11 and 11 (T>A) at D7S820 are depicted.

FIGS. 2A-K present results obtained from genotyping of the 11 STR markers showing nucleotide variability within an Austrian population samples. The frequency data on the left of the figures representing CE data and that on the right the ICEMS data of the Examples.

FIG. 3 presents the properties of 21 STRs commonly used in forensic genetics.

FIG. 4 presents the observed allelic frequencies of STRs showing length and nucleotide variability.

FIG. 5 presents the results obtained from sequencing a selected number of SE33 alleles.

FIG. 6 presents results obtained from sequencing a selected number of D2S1338 alleles.

FIG. 7 presents results obtained from sequencing a selected number of vWA alleles.

FIG. 8 presents results obtained from sequencing a selected number of D21 S11 alleles.

FIG. 9 presents results obtained from sequencing a selected number of D3S1358 alleles.

FIG. 10 presents results obtained from sequencing a selected number of D16S539 alleles.

FIG. 11 presents results obtained from sequencing a selected number of D8S1179 alleles.

FIG. 12 presents results obtained from sequencing a selected number of D7SB20 alleles.

FIG. 13 presents results obtained from sequencing a selected number of D13S317 alleles.

FIG. 14 presents results obtained from sequencing a selected number of D5S818 alleles.

FIG. 15 presents results obtained from sequencing a selected number of D2S441 alleles.

### DESCRIPTION OF VARIOUS EMBODIMENTS AND EXAMPLES

Aspects of the present teachings may be further understood in light of the following discussion and examples, which are not exhaustive and which should not be construed as limiting the scope of the present teachings in any way. Prior to further describing the present teachings, it may be helpful to provide an understanding thereof to set forth abbreviations and definitions of certain terms to be used herein.

As used herein, the article “a” is used in its indefinite sense to mean “one or more” or “at least one.” That is, reference to any element of the present teachings by the indefinite article “a” does not exclude the possibility that more than one of the elements is present.

As used herein, STR serves as an abbreviation for “short tandem repeat(s),” a short DNA sequence (typically 2 to about 10 bases long) polymorphism that repeats itself in tandem.

As used herein, SNP serves as an abbreviation for “single nucleotide polymorphism(s),” a DNA sequence variations that occur when a single nucleotide in the genome sequence is altered.

As used herein, the abbreviation SNPSTR refer to a genetic marker which combines a STR marker with one or more tightly linked SNPs. In various embodiments, SNPSTRs which contain a SNP and a STR between about 100 to about 500 bp apart are used.

In various aspects, the present teaching provides methods for determining nucleic acid length and sequence variation, for example, between an unknown sample and a reference sample. In various embodiments, a method amplifies one or more specific regions of an oligonucleotide molecule in a sample comprising oligonucleotide molecules to produce a sample of amplicons, the sample of amplicons comprising two or more different amplicons. In various embodiments, two or more specific regions are amplified. In various embodiments, the methods (i) denature the amplicons in the sample of amplicons to produce a first set of single-stranded amplicons and a second set of single-stranded amplicons, single stranded amplicons of the second set being complementary to the corresponding single stranded amplicons of the first set and (ii) subject the first and second set of single-stranded amplicons to mass spectrometric analysis to obtain the masses of the amplicons in the first and second set of single-stranded amplicons. The masses of the amplicons are then used, at least in part, to determine nucleic acid length and sequence variation.

For example, referring to Table 1, in various embodiments the measured molecular masses of the first and second set of amplicons are compared to the mass(es) expected and/or calculated for one or more reference nucleic acid sequences. Table 1 summarizes mass differences (in amu) observed for various sequence variations and substitutions. In various embodiments, the composition of two sequences (e.g., amplicon vs. reference, amplicon vs. amplicon) is considered substantially identical if the molecular mass difference is smaller than the typically observed mass measurement error. In various embodiments, molecular mass differences exceeding the typically observed measurement error indicate the presence of a sequence variation (variation either in length or nucleotide composition).

TABLE 1
Mass difference information observed for sequence variations.
Units of mass are in atomic mass units (amu).

| | | C | T | A | G |
|---|---|---|---|---|---|
| | | insertion/deletion of | | | |
| mass difference | | ±289.182966 | ±304.194376 | ±313.20781 | ±329.20724 |
| | | substituted by | | | |
| Original | C | 0 | 15.0114 | 24.0248 | 40.0243 |
| base | T | −15.0114 | 0 | 9.0134 | 25.0129 |
| | A | −24.0248 | −9.0134 | 0 | 15.9994 |
| | G | −40.0243 | −25.0129 | −15.9994 | 0 |

In various embodiments, the methods determine variation between amplicon and a reference sequence. In various embodiments, the methods determine variation between amplicons of the same specific region of the oligonucleotide molecule.

In various embodiments, a sequence variation detected in the first set of single-stranded amplicons is compared to the sequence variation detected in the second set of single-stranded amplicons complimentary to the first, to determine and/or confirm the sequence variation based on the sequence variation in the first set being substantially complimentary to the sequence variation in the second set. For example, for complimentary amplicons, the second set nucleotide composition (AₖCₗGₘTₙ) is complementary to the first set nucleotide composition (AₙCₘGₗTₖ).

In various embodiments, the methods distinguish between alleles having substantially the same length in the oligonucleotide based at least on nucleic acid sequence variation. Accordingly, in various versions of various embodiments, sub-allelic variations can be determined.

In various aspects, sequence variability can be determined by generating from the measured masses of the amplicons in the first and second set of single-stranded amplicons a list of possible nucleotide compositions. In various embodiments, the second set nucleotide composition (AₖCₗGₘTₙ) is complementary to the first set nucleotide composition (AₙCₘGₗTₖ). These possible nucleotide compositions can be compared to the nucleotide compositions of one or more reference nucleic acid sequences to determine the nucleic acid sequence variation. For example, in various embodiments, the methods generate a list of paired candidate sequences based on masses of the first and second set of amplicons, one member of the pair corresponding to a candidate sequence for the first set of amplicons and the other member of the pair corresponding to a candidate sequence for the second set of amplicons, and where the sequence pairs are complimentary; and determine the length and nucleic acid sequence variation of a specific amplified region by comparing the candidate sequences to a reference nucleic acid sequence.

A variety of sequence information can be determined in various embodiments of the present teachings. For example, in various embodiments, variations comprising one or more of a single nucleotide polymorphism (SNP), a short tandem repeat variation (STR), and SNPSTR, can be determined. In various embodiments of determination of a SNPSTR, a variation having a SNP and STR spacing of one or more of greater than about 100 bp, greater than about 200 bp and greater than about 500 bp can be determined. In various embodiments of determination of a SNPSTR, a variation having a SNP and STR spacing of one or more of less than about 100 bp, less than about 200 bp and less than about 500 bp can be determined. In various embodiments of determination of a SNPSTR, a variation having a SNP and STR spacing in the range between about 50 bp to about 500 bp can be determined.

A wide variety of oligonucleotide molecules can be analyzed with various embodiments of the present teachings including, but not limited to, deoxyribonucleic acid (DNA) or a fragment thereof. A variety of DNA can be analyzed including, but not limited to, mitochondrial DNA, nuclear DNA, bacterial DNA, viral DNA, a fragment thereof, and combinations thereof.

A variety of PCR techniques can be used to provide amplicons. In various embodiments, the amplification step comprises using amplification primers that are shifted closer to the repeat region, e.g., to facilitate increasing discrimination in degraded DNA samples. In various embodiments, use of primers closer to the repeat region facilitates capturing the sequence variability of the repeat region and facilitates increasing the number of discriminative allele variants observed, which in various embodiments, e.g., can lead to an overall increased forensic efficiency. In various embodiments, the amplification is selected to produce amplicons having less than about 500 bp, less than about 250 bp; less than about 100 bp; less than about 75 bp; and/or less than about 50 bp. In various embodiments, the amplification is selected to produce amplicons having a length in the range between about 50 bp to about 150 bp; between about 50 bp to about 250 bp; between about 100 bp to about 3000 bp; and/or between about 50 bp to about 500 bp.

In various embodiments, the step of amplifying one or more specific regions of an oligonucleotide molecule in a sample uses a multiplex-PCR approach to generate amplicons in a multiplex fashion. For example, in various embodiments, the step of amplifying comprises amplifying at least two or more specific regions of an oligonucleotide molecule in the sample; amplifying at least four or more specific regions of an oligonucleotide molecule in the sample; amplifying at least eight or more specific regions of an oligonucleotide molecule in the sample; amplifying at least twelve or more specific regions of an oligonucleotide molecule in the sample; amplifying at least sixteen or more specific regions of an oligonucleotide molecule in the sample; and/or amplifying at least twenty-four or more specific regions of an oligonucleotide molecule in the sample.

In various embodiments, liquid chromatography (LC) can be used to prefractionate mixtures of oligonucleotide molecules, amplicons, or both, to, for example, reduce the number of species simultaneously introduced into the mass spectrometer facilitating their mass spectrometric detection. In various embodiments, a step of LC can be used to substantially simultaneously characterize amplicons produced within different PCRs. For example, different amplicons from different genomic locations or from the same genomic location but from different individuals can be co-loaded onto the same column enabling their simultaneous characterization within one single LC run, which, for example, can facilitate reducing the overall analysis time.

A variety of techniques can be used to denature the amplicons, including but not limited to thermal (e.g., loading at least a portion of the sample of amplicons on a chromatographic column; and heating the chromatographic column to denature the amplicons), chemical (e.g., treatment with sodium hydroxide), enzymatic, and combinations thereof.

A wide variety of mass spectrometric instruments and analysis techniques can be used to obtain the masses of the amplicons including, but not limited to, matrix-assisted laser desorption-ionization mass spectrometry (MALDI-MS) (P. L. Ross et al. (1997) Anal. Chem 69: 3699-3972; J. M. Butler et al. (1998) Int. J. Legal Med. 112: 49) and electrospray ionization mass spectrometry (ESI-MS) (J. C. Harmis et al. (1999) Rapid Commun. Mass Spectrom 13: 954-962; S. Hahner et al. (2000) Nuc Acids Res. 28: e82; H. Oberacher et al. (2001) Anal. Chem. 73: 5109-5115; J. C. Hannis et al. (2001) Mass Spectrom 15: 348-350).

In various embodiments, use is made of instruments with a mass measurement error of less than about 50 ppm, and/or in the range between about 20 ppm to about 50 ppm. In various embodiments, the mass analyzer comprise one or more of a quadrupoles, RF multipoles, ion traps, time-of-flight (TOF), TOF in conjunction with a timed ion selector, and Fourier transform ion cyclotron resonance (FTICR).

### EXAMPLES

In the present Examples, characterization of STR alleles using various embodiments of the present teachings was conducted with ion-pair reversed-phase high-performance liquid chromatography ESI-MS (ICEMS) (H. Oberacher et al. (2001) Angew. Chem. Int Ed. 40: 3828-3830; H. Oberacher et al. (2005) Anal. Chem. 77: 4999-5008; H. Oberacher et al. (2006) Anal. Bioanal. Chem. 384: 1155-1163; H. Oberacher et al. (2006) Anal. Bioanal. Chem. 386: 83-91; H. Oberacher et al. (2006) Anal. Chem. 78: 7816-7827; H. Oberacher et al. (2007) Int. J. Legal Med. 121: 57-67a). The data of these Examples using various embodiments of the present teaching are indicated by the abbreviation ICEMS. For convenient and concise reference when discussing data obtained by various embodiments of the present teachings such data is often referred to as ICEMS, ICEMS results, ICEMS data, ICEMS technique, etc. It is to be understood that this use of the abbreviation ICEMS is not intended to limit the present teachings to use of an ICEMS instrument or limit the present teachings any other way.

The selection of markers in these Examples was not necessarily restricted to the motif structure or the vicinity of known SNPs; we investigated STR loci (Table 2) that are widely used in the forensic community and therefore of interest for forensic comparison with established sets of data (e.g. database searches).

TABLE 2
STR loci used in the Examples.
STR locus

SE33
D2S1338
vWA
D21S11
D3S1358
D16D539
D8S1179
D7S820
D13S317
D5S818
D2S441
D19S433
FGA
D18S51
CSF1PO
PentaD
PentaE
TH01
TPOX
D10S1248
D22S1045

All 21 STR-loci mentioned in Table 2 were amplified in an Austrian population sample consisting of 92-99 unrelated individuals using primer sequence information from the literature (P. Grubwieser et al. (2007) Int. J. Legal Med. 121: 85-89; J. M. Butler et al. (2003) J Forensic Sci. 48: 1054-1064). The primers for the amplification of D19S433 were newly designed. The resulting amplicon lengths as extracted from the Ensemble database were in the range between 79 and 246 bp and therefore facilitated unequivocal detection of many kinds of single base exchanges even within heterozygous samples. For each marker, reference sequences corresponding to putative length variants were obtained by adding/deleting one or more building blocks to/from the database sequence. We used these reference sequences to calculate theoretical molecular masses corresponding to the blunt-ended and monoadenylated forward and reverse single-strands.

The allelic state(s) of a sample were determined by measured molecular masses when compared with the whole ensemble of calculated masses. First the length and therewith the number of repeat units of the sample allele(s) were determined by searching the closest matching length variant(s). Subsequently, additionally existing nucleotide changes were identified. Deviations between the measured and the theoretical masses larger than the routinely observed measurement error (20-50 ppm) were taken in these Examples to indicate the presence of some kind of nucleotide exchange relative to the equally sized reference sequence. The values of the observed mass-differences were used to predict the kinds of nucleotide exchanges. In these Examples, both DNA strands were used as the basis for the assignment of the mass spectrometric screening assay, thus increasing the reliability of the allele notation. Using the methods of the present teachings, for 11 of the tested 21 STR loci (SE33, D2S1338, vWA, D21 S11, D3S1358, D16S539, D8S1179, D7S820, D13S317, D5S818 and D2S441), additional allele variants were observed which were not observed with CE analysis.

The established nomenclature rules were used for calling alleles identified via electrophoretic sizing experiments (W. Bar et al. (1994) Int. J. Leg. Med. 107: 159-160): (1) alleles should be designated by the number of repeats they contain even if the sequence of the repeats is different; (2) when an allele does not conform to the standard repeat motif of the system in question, it should be designated by the number of complete repeat units and the number of base pairs of the partial repeat; and (3) these two values should be separated by a decimal point.

The disclosure of nucleotide variability of STR markers determined by application of various embodiments of the present teachings, however, calls for an adjustment of the allele nomenclature because of the additional information obtainable. The report of measured molecular mass(es) or derived nucleotide compositions would represent one possible way of allele calling. Alternatively, the putative length of the repeat unit together with the mass differences relative to the corresponding reference sequence could be used to unequivocally describe the ICEMS results. Here, we apply the latter method as it can be more readily compared to the already existing STR nomenclature, and would be less susceptible to differences introduced by different primer locations.

To facilitate application of the data of the Examples within the forensic community, the observed mass deviations were converted into putative nucleotide substitution(s) within the sequence of the forward single strand. For example, for an allele of D7S820 a molecular mass of 49597 was measured for the forward strand and 49107 for the reverse strand. These masses approximated the masses of an allele consisting of 11 repeat units (49588, 49116). Mass deviations of ±9 mass units or 181 ppm indicated the presence of a T>A polymorphism. Thus, this distinct allele was called H(T>A). It is be understood that this nomenclature can be compared with the established STR nomenclature by deleting the additional nucleotide variability, hence, facilitating the use of information obtained by practice of various embodiments of the present teachings with the huge amount of already examined DNA fingerprints for DNA profiling.

In these Examples, three different sets of experiments were conducted.

In a first set of experiments all STR alleles typed by ICEMS were characterized with CE using appropriate STR typing kits. With the exception of D19S433, the ICEMS results were consistent with the CE results. In D19S433, CE-generated alleles were generally two repeat units larger as proposed by ICEMS. The seeming difference can be explained by the number of (AAGG)-blocks that are included as repeat-units by the manufacturer of the applied STR typing kit and the operators of “STRBase” (C. M. Ruitberg et al. (2001) Nucleic Acids Res. 29: 320-322).

In a second set of experiments a representative number of alleles of all 11 STR markers that showed nucleotide variability were amplified with a dNTP mixture containing dUTP instead of dTTP to produce a different set of amplicons. Uracil and thymine are both complementary to adenine. Hence, with the exception of the primer nucleotides all deoxythymidines were exchanged by deoxyuridines within the amplicons. The molecular mass of deoxyuridine is approximately 15 mass units smaller than that of deoxythymidine. Depending on the kind of dNTP-mixture, different molecular mass deviations were measured for sequence alterations in which deoxythymidines/deoxyuridines were involved. Thus, nucleotide variations could be well defined that would hardly be distinguishable from each other under traditional approaches i.e. A< >G and C< >T, C< >A and T< >G changes. In addition, the detection of (A< >T)-polymorphisms within heterozygous samples was facilitated.

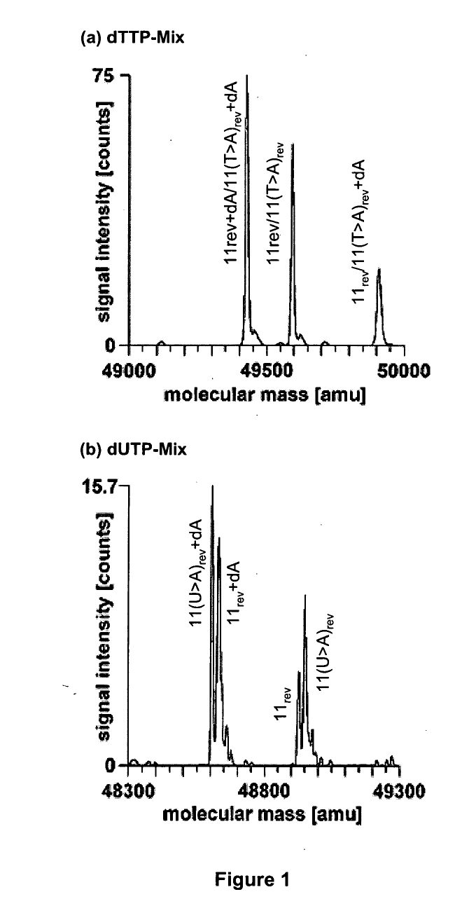

For example, referring to FIGS. 1A-B, ICEMS results obtained from two different PCR amplifications of a sample harbouring the alleles 11 and 11 (T>A) at D7S820 are depicted. The allele-specific single strands remained unresolved as long as the dTTP-mixture was used for PCR (FIG. 1A). Molecular mass deviations that were larger than the usually observed measurement errors indicated the simultaneous presence of two sequence variants (H. Oberacher et al. (2006) Anal. Bioanal. Chem. 386: 83-91). In these Examples the application of dUTP led to a clear separation of the two sequence variants (FIG. 1B).

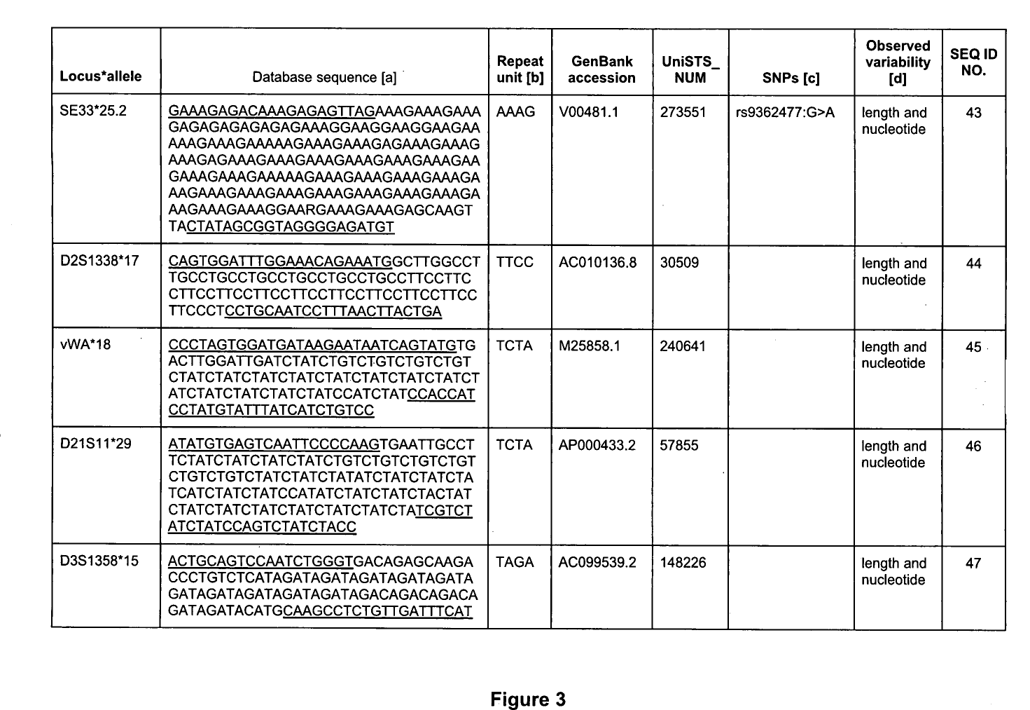

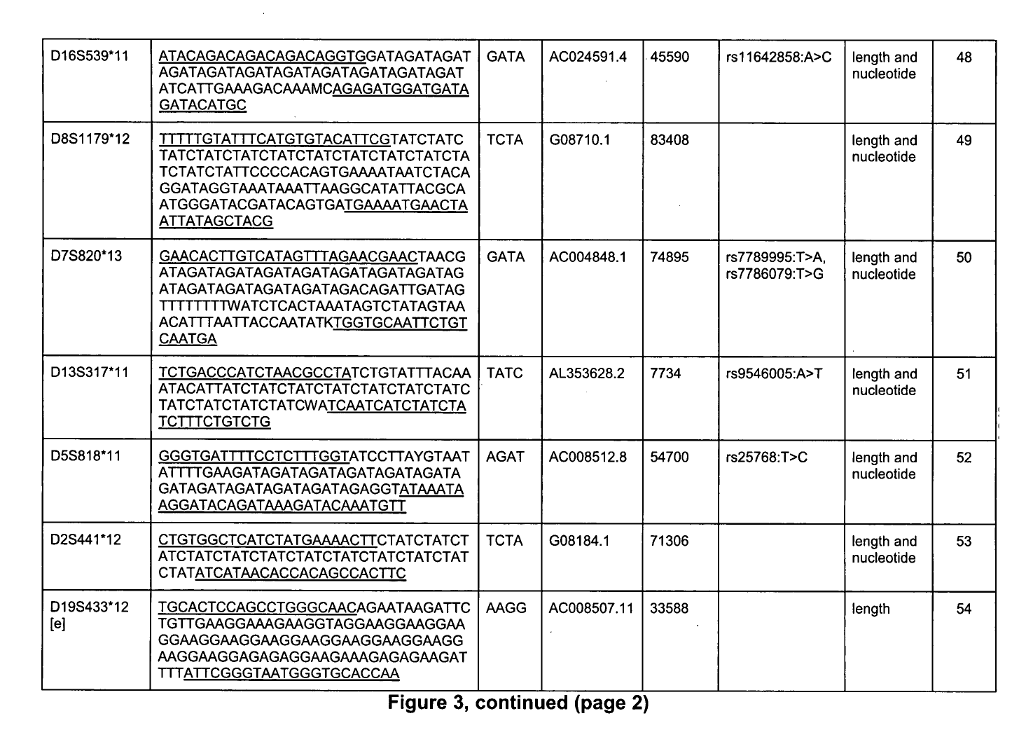

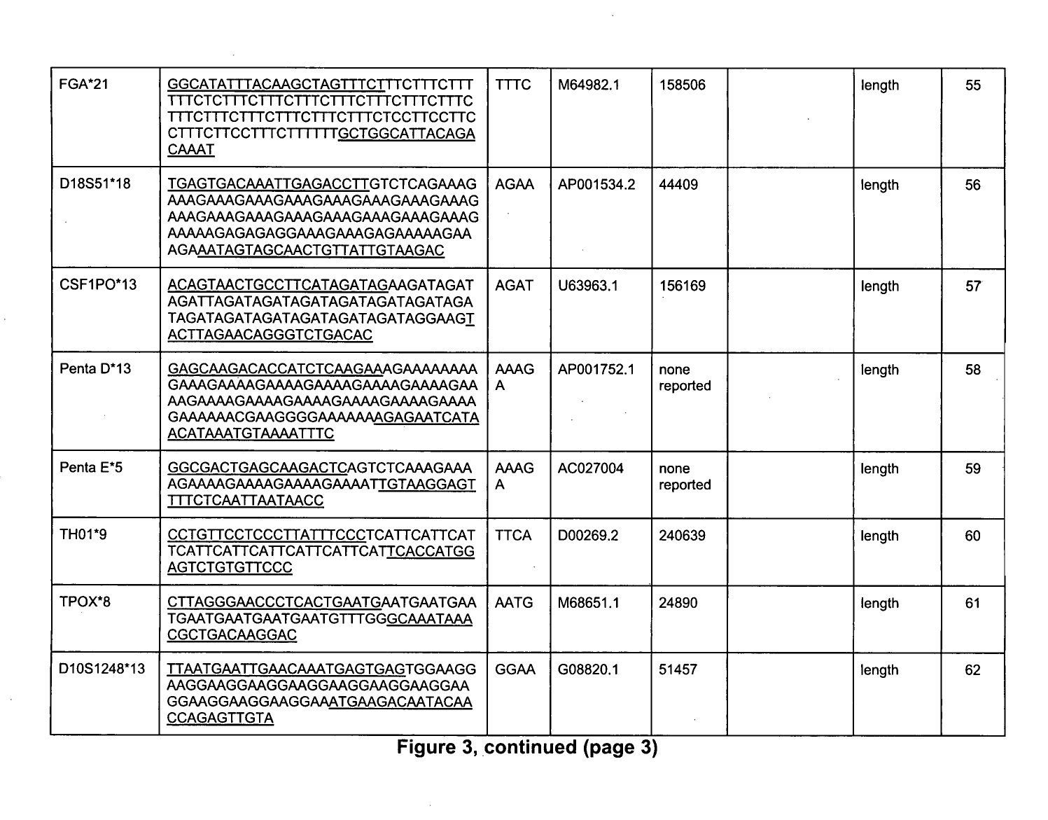

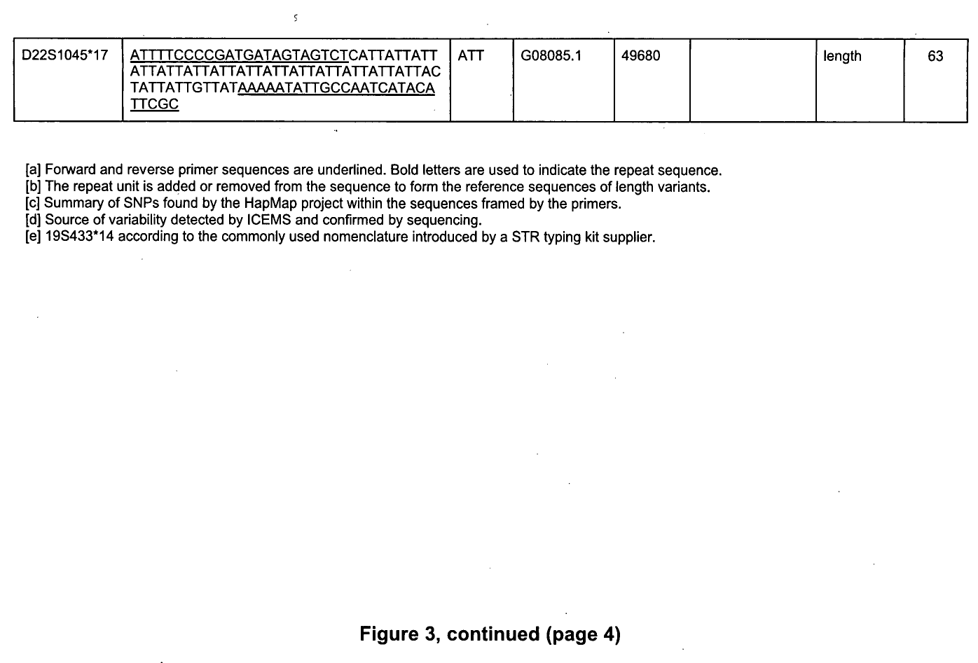

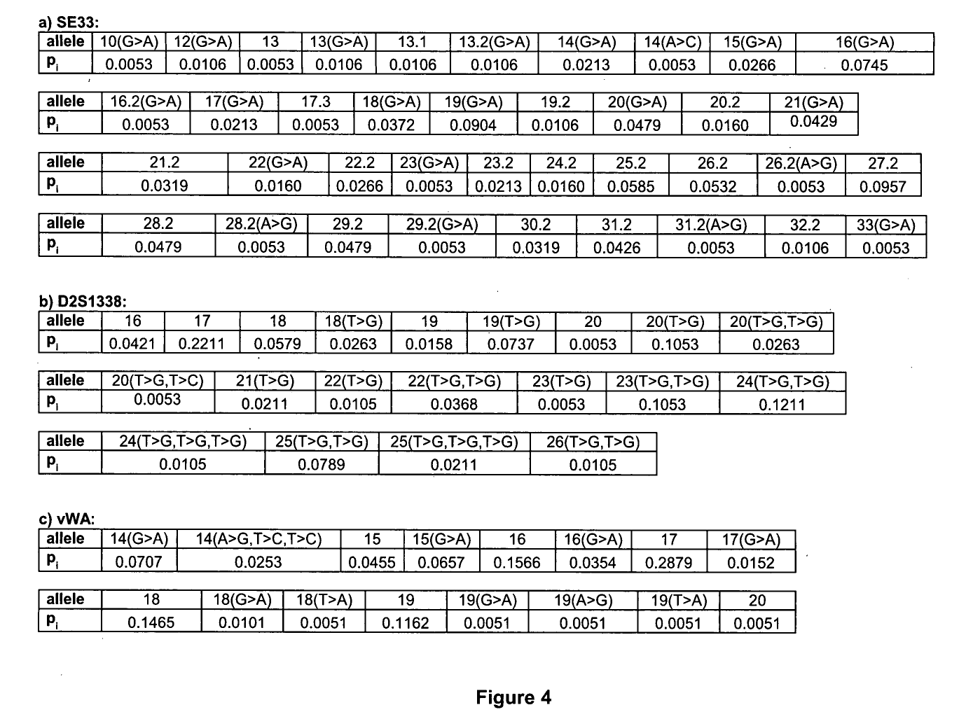

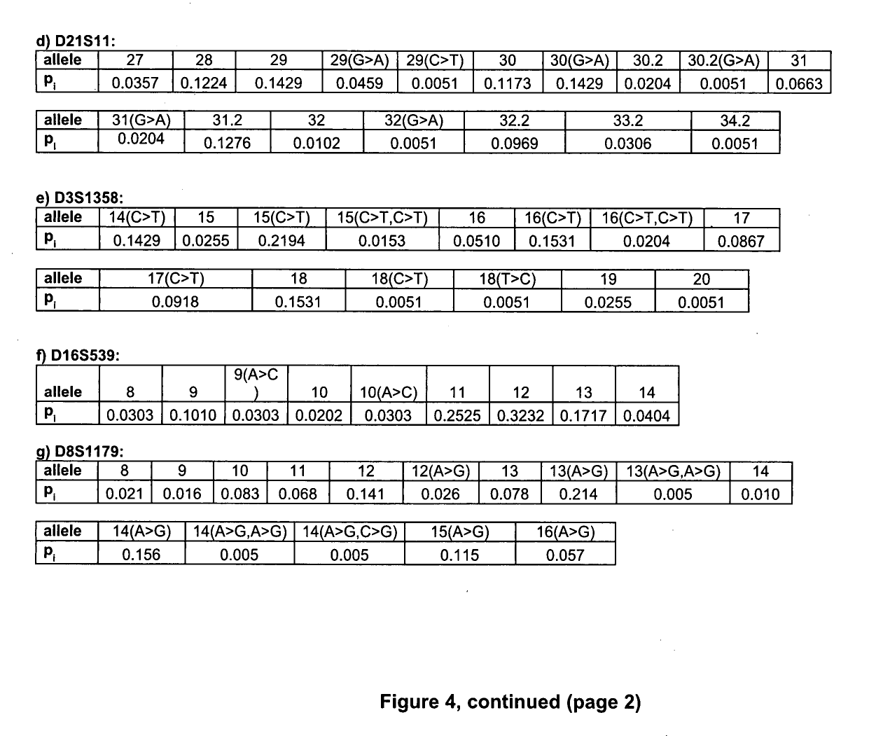

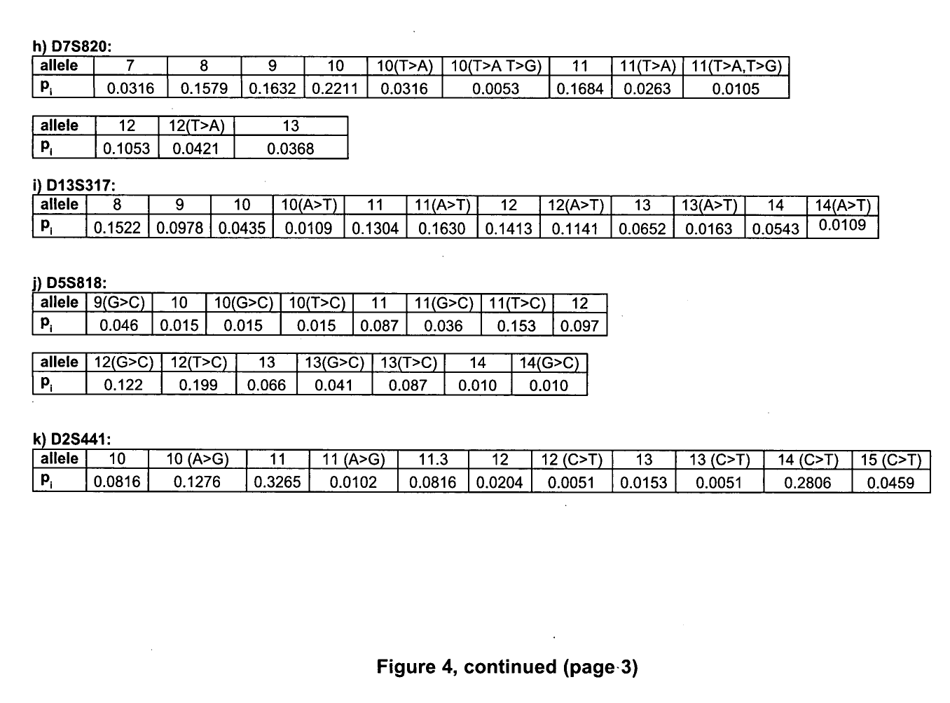

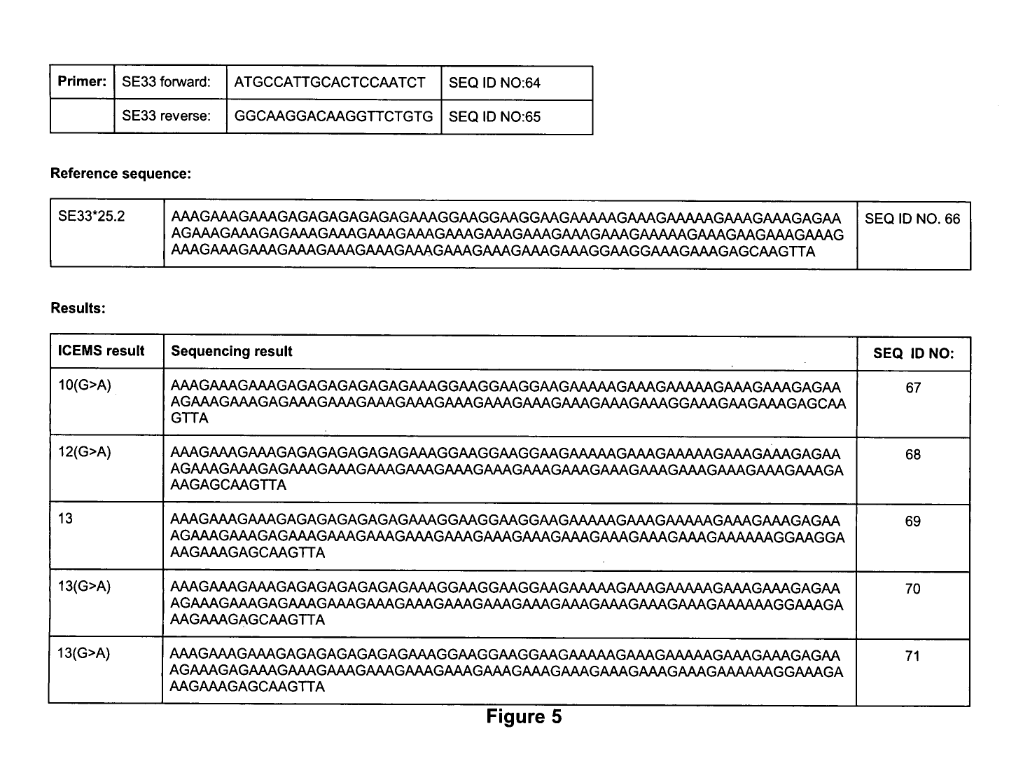

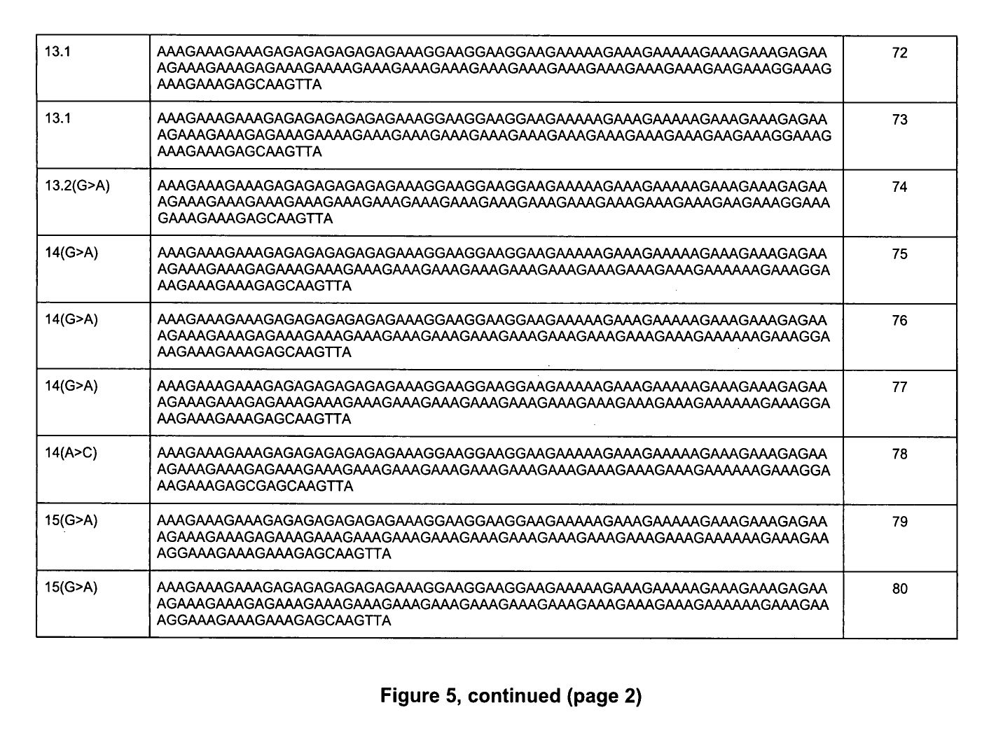

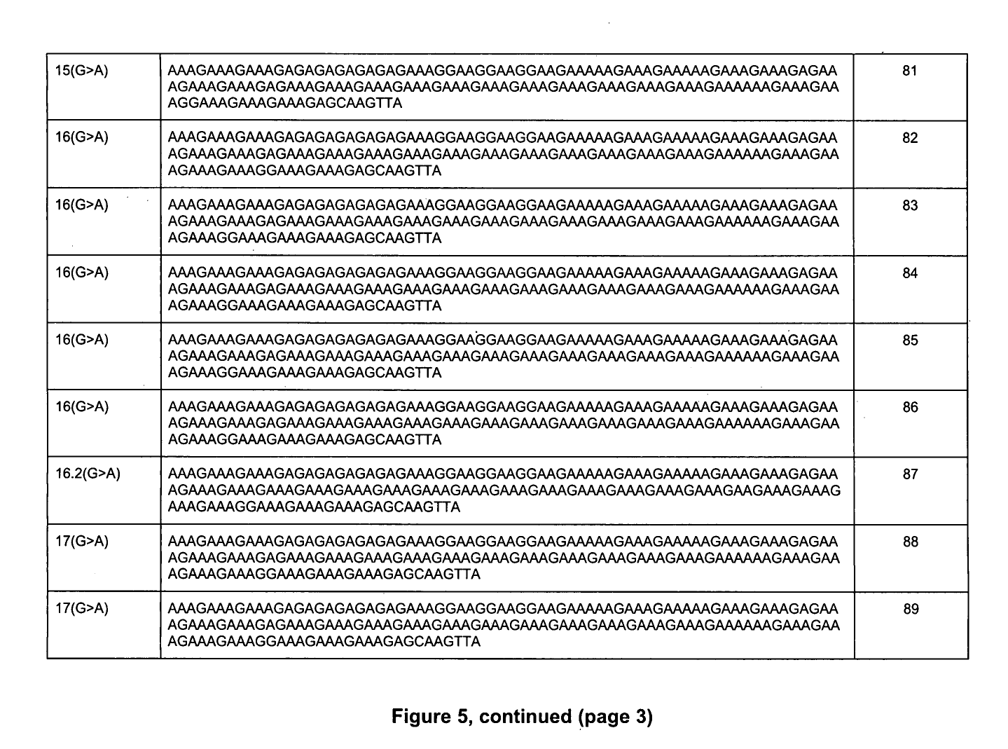

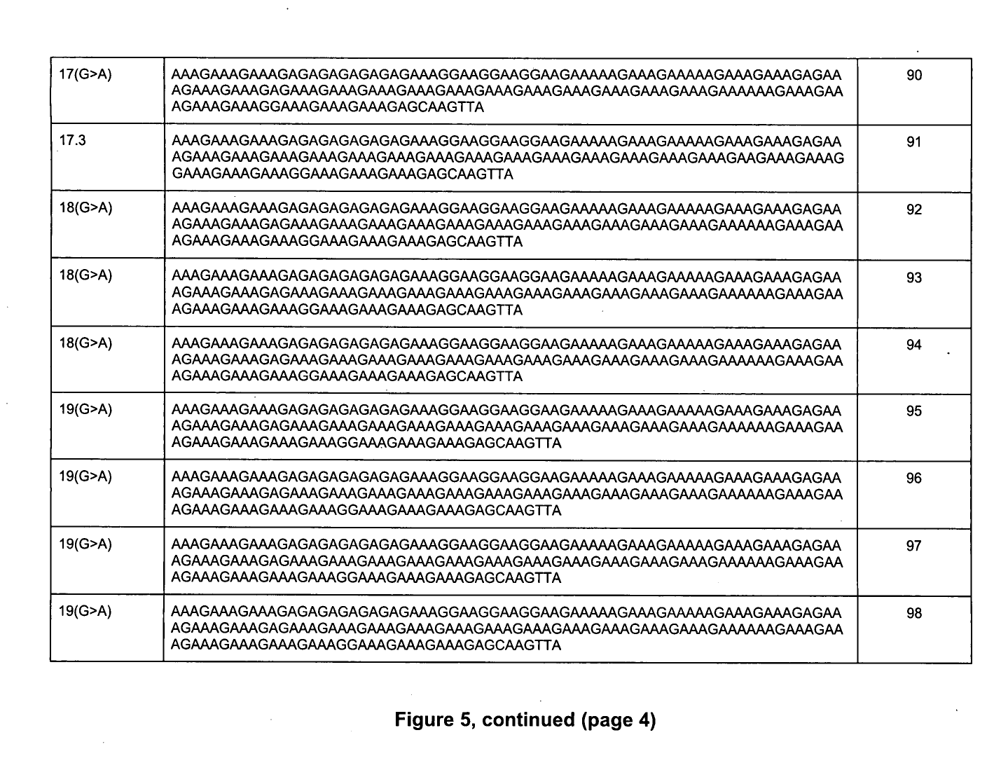

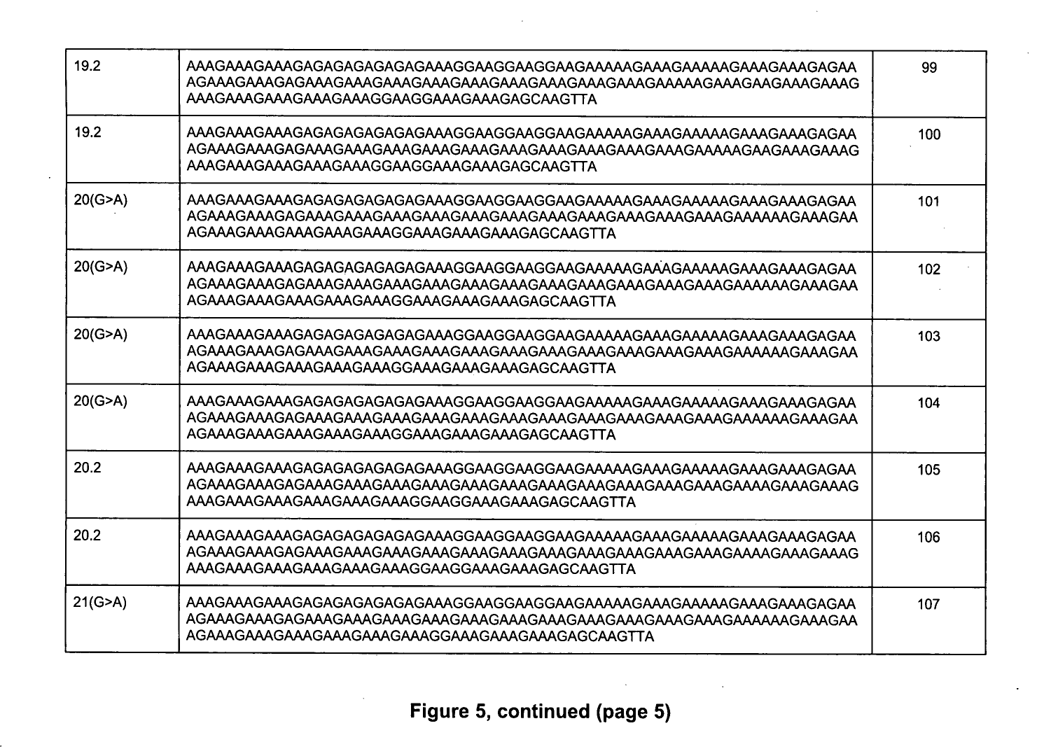

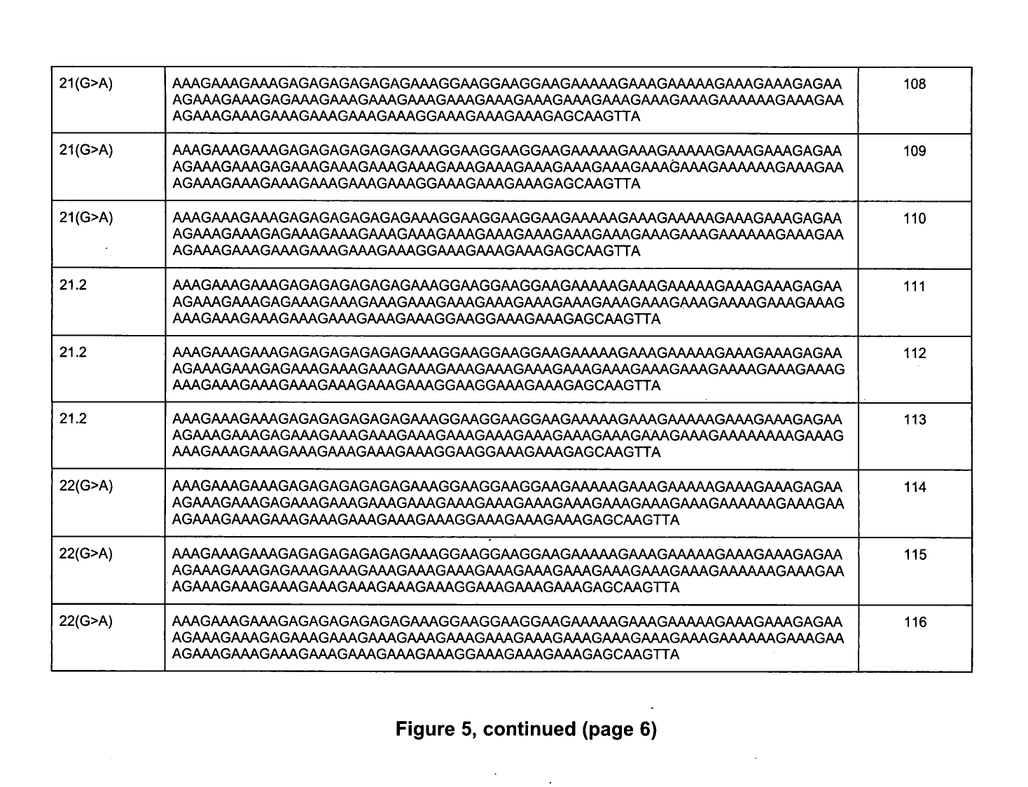

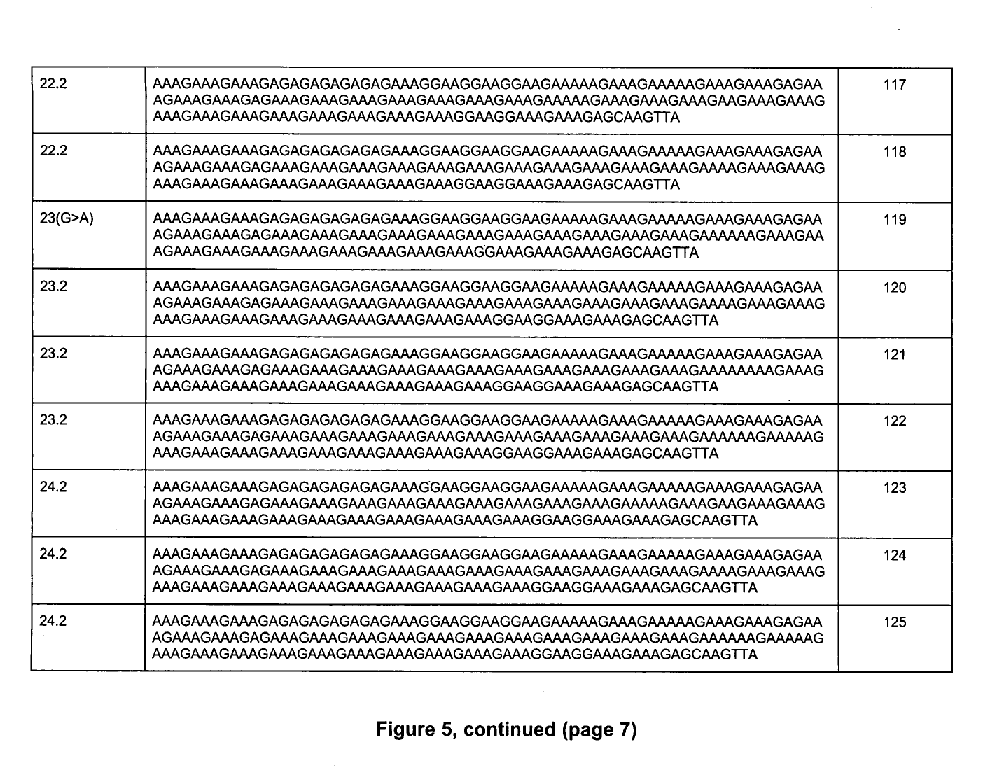

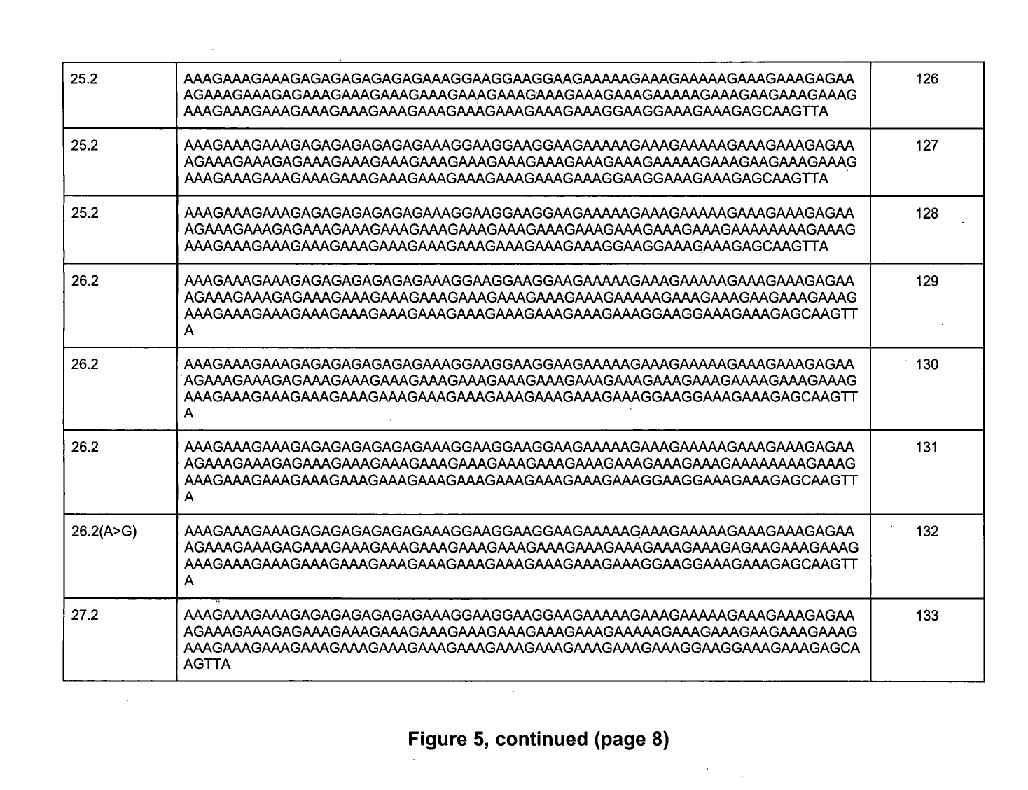

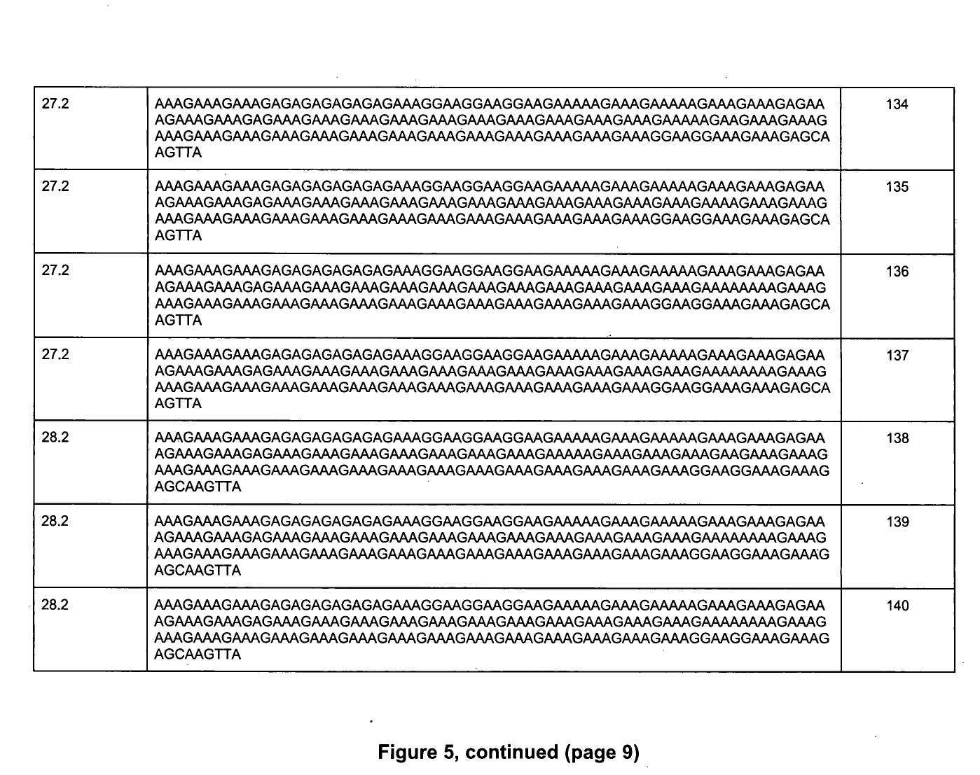

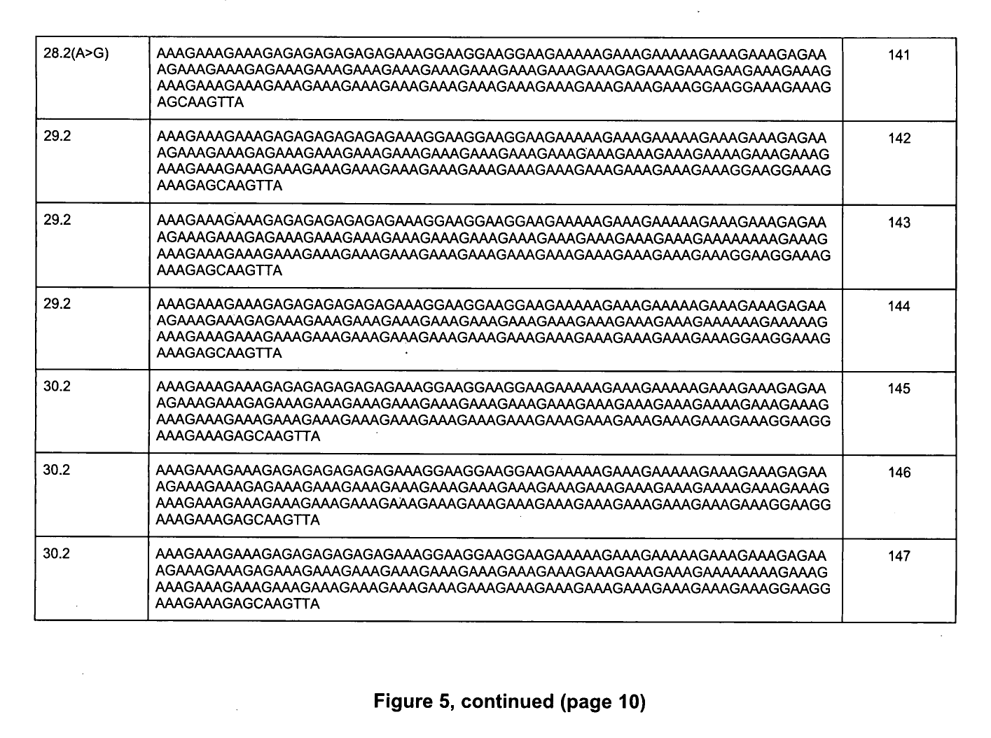

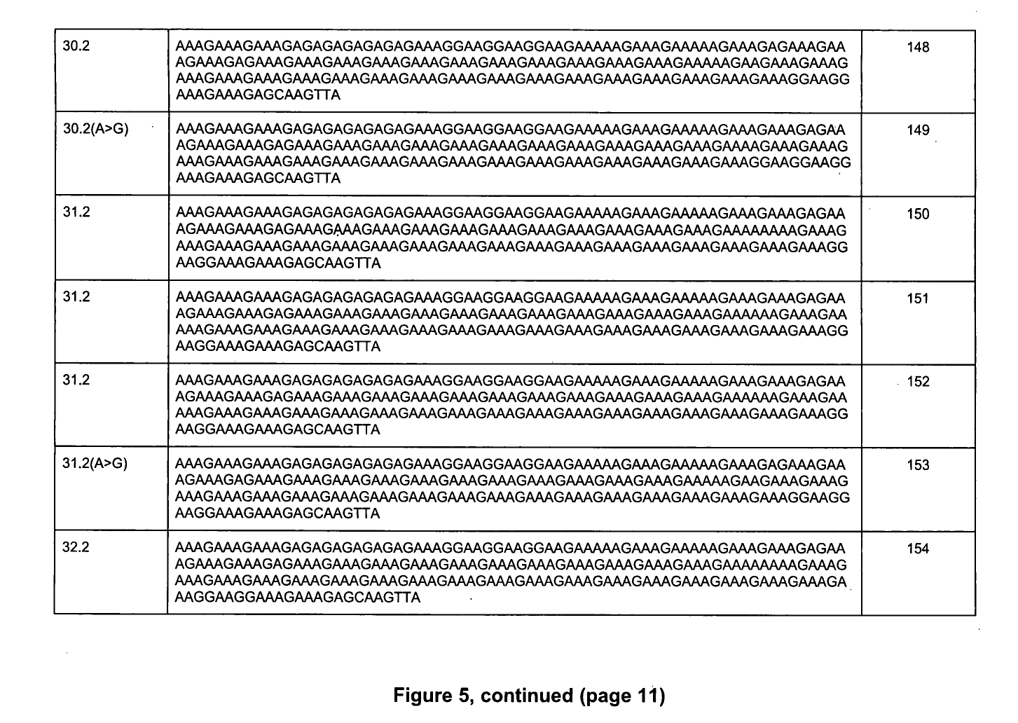

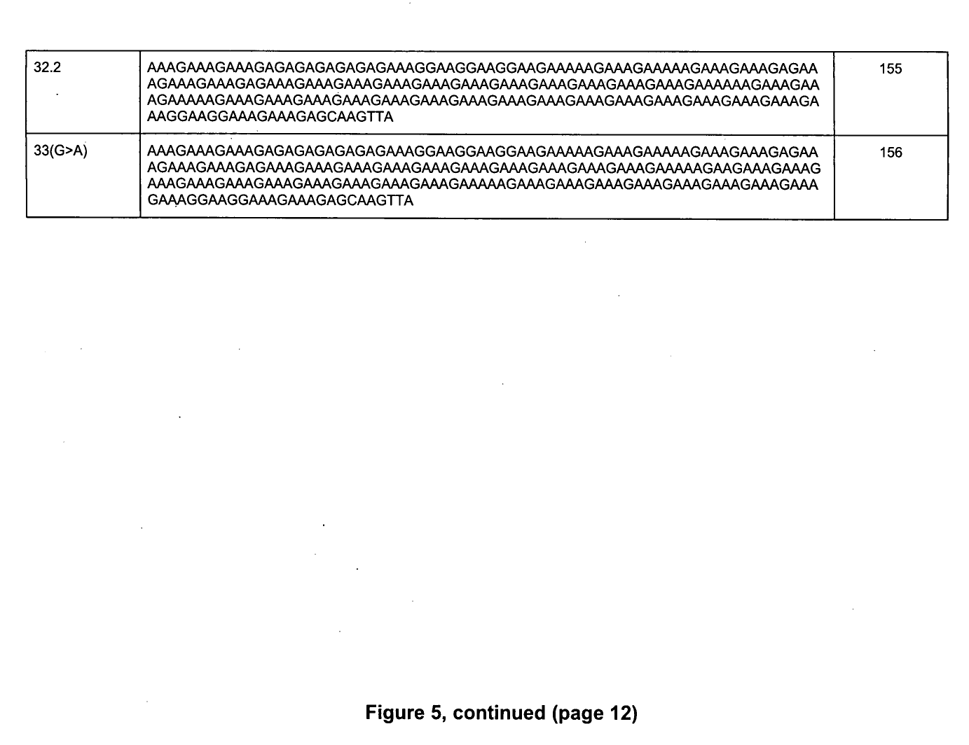

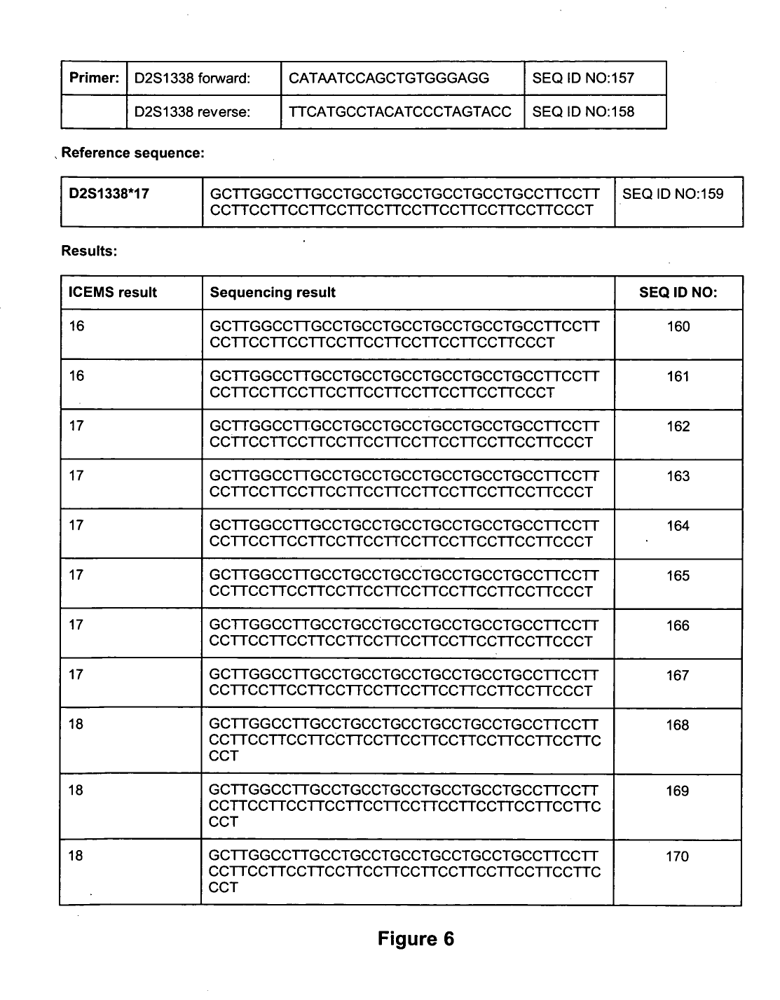

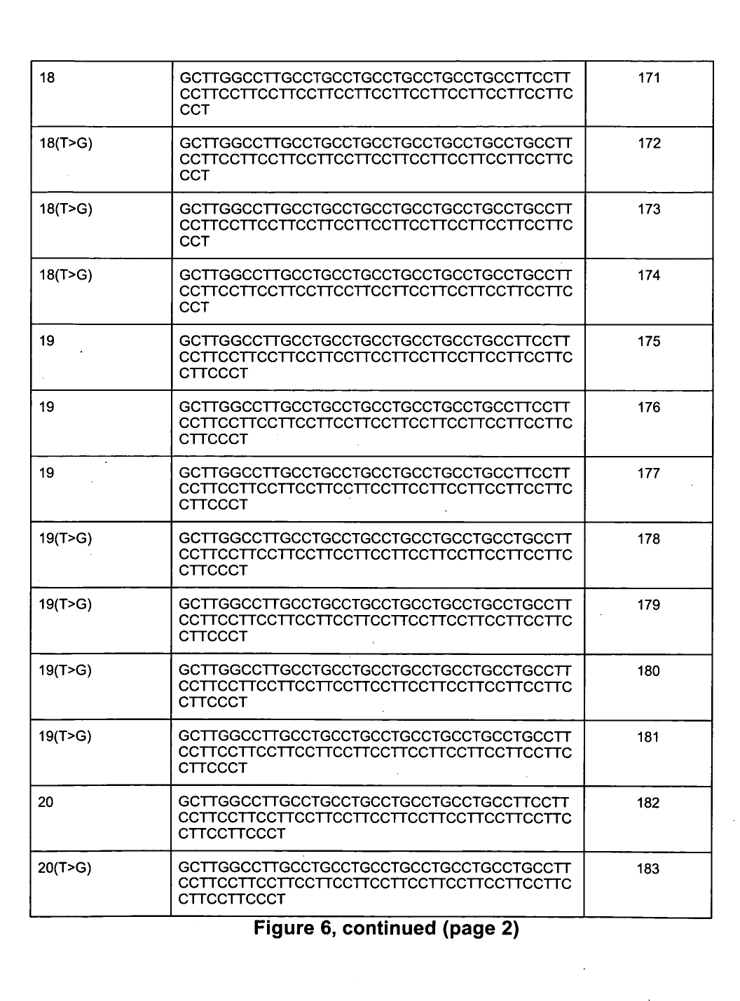

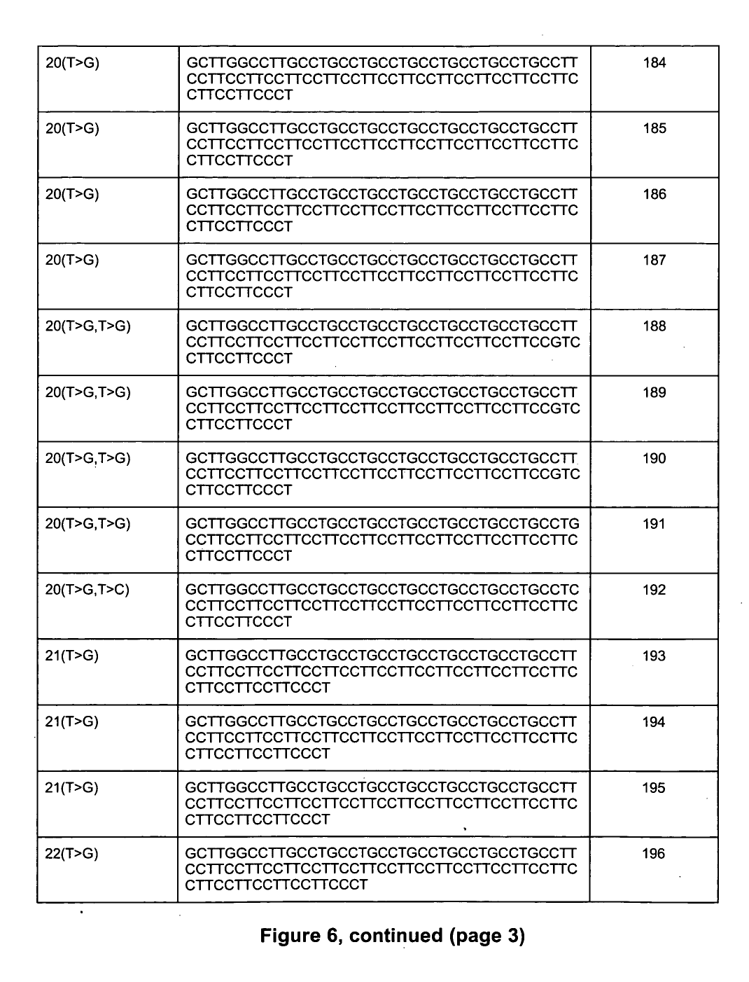

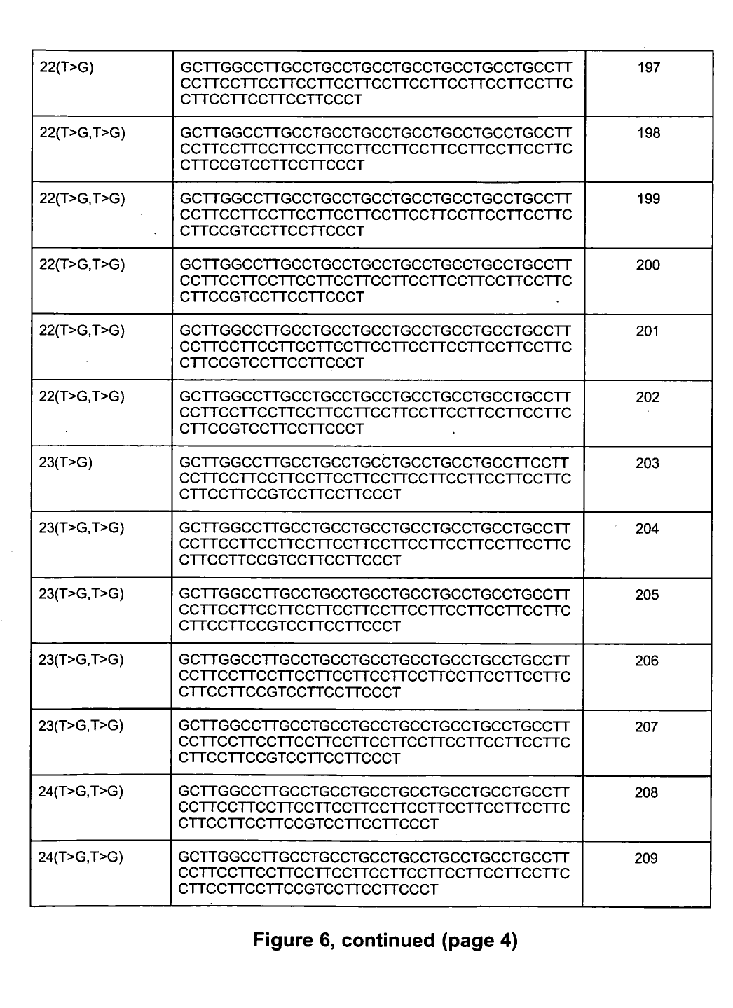

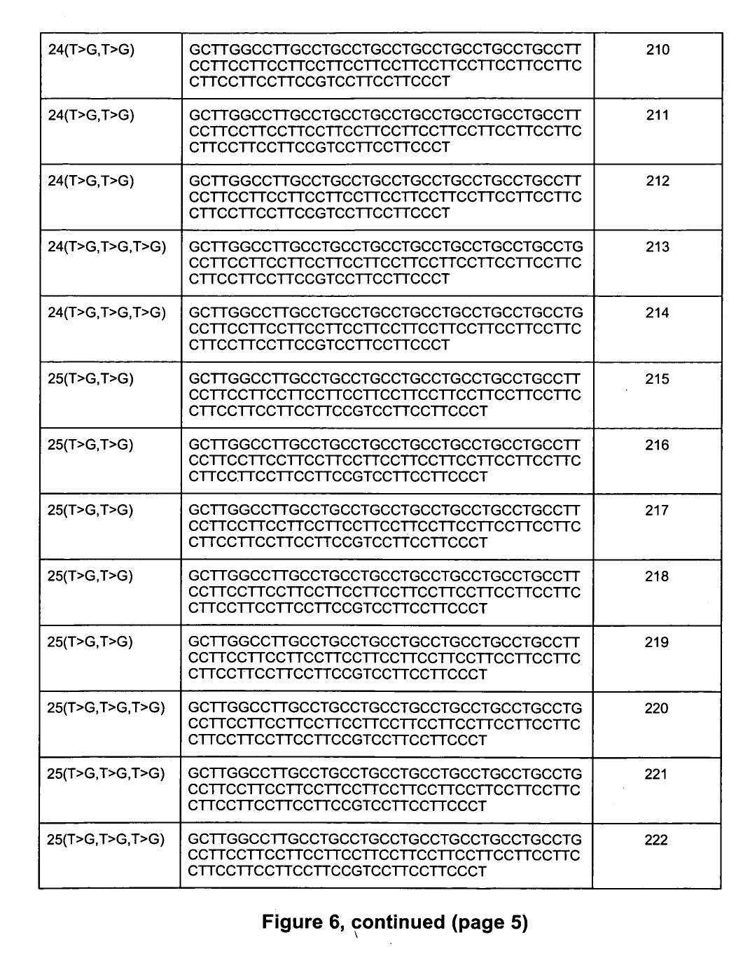

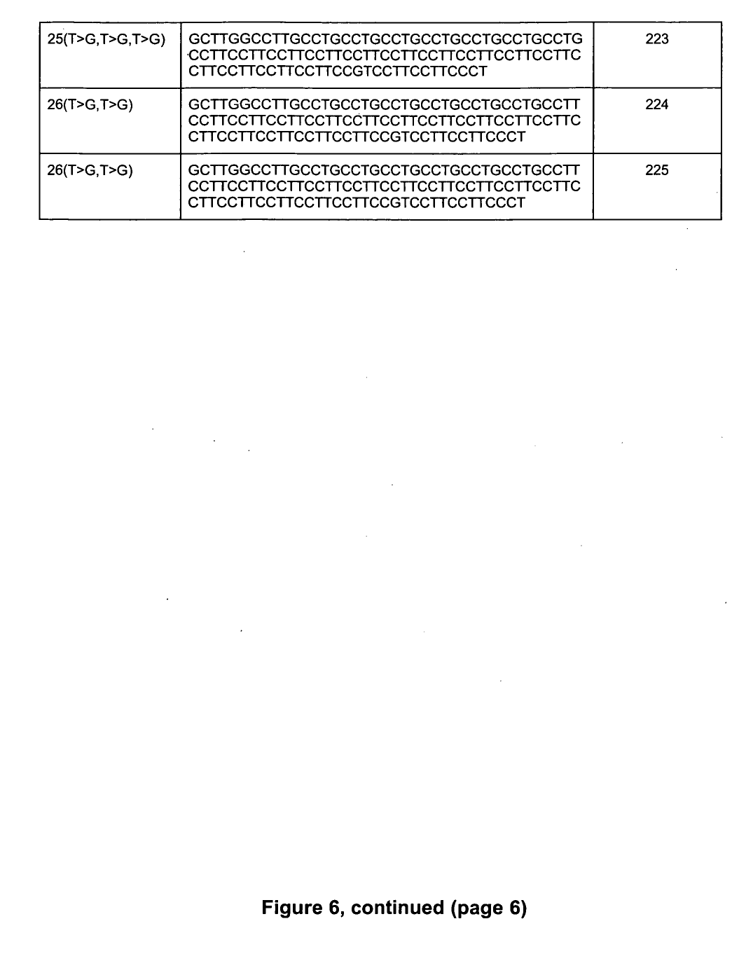

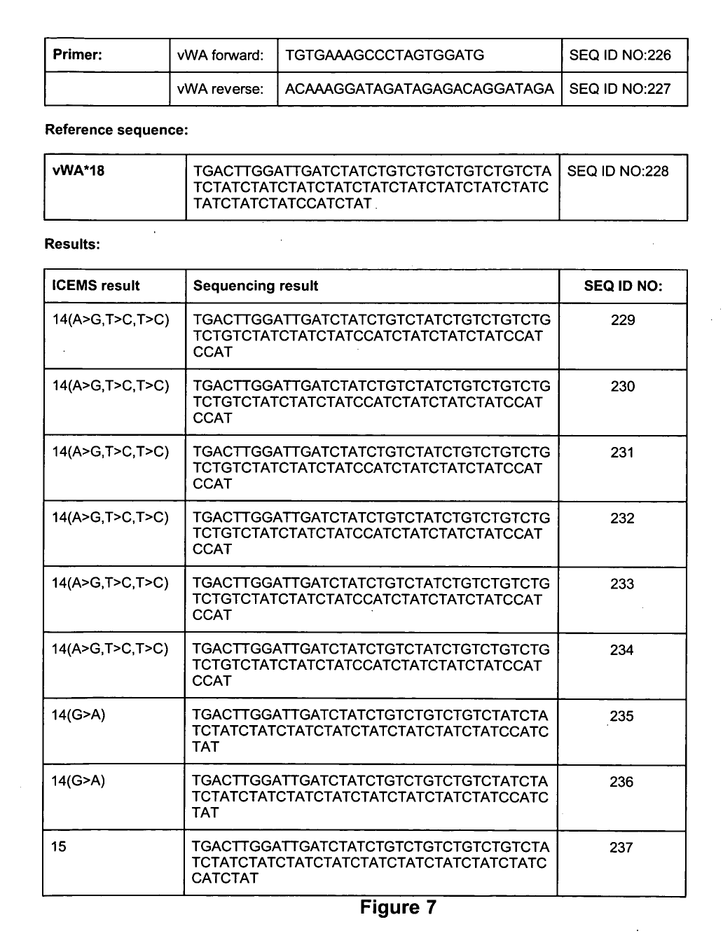

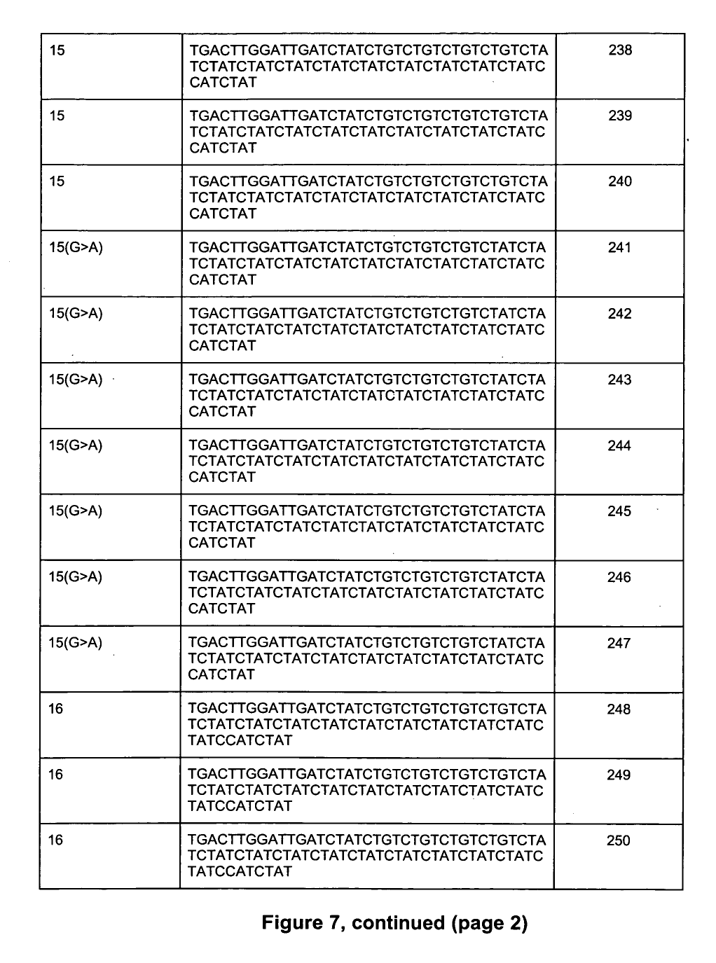

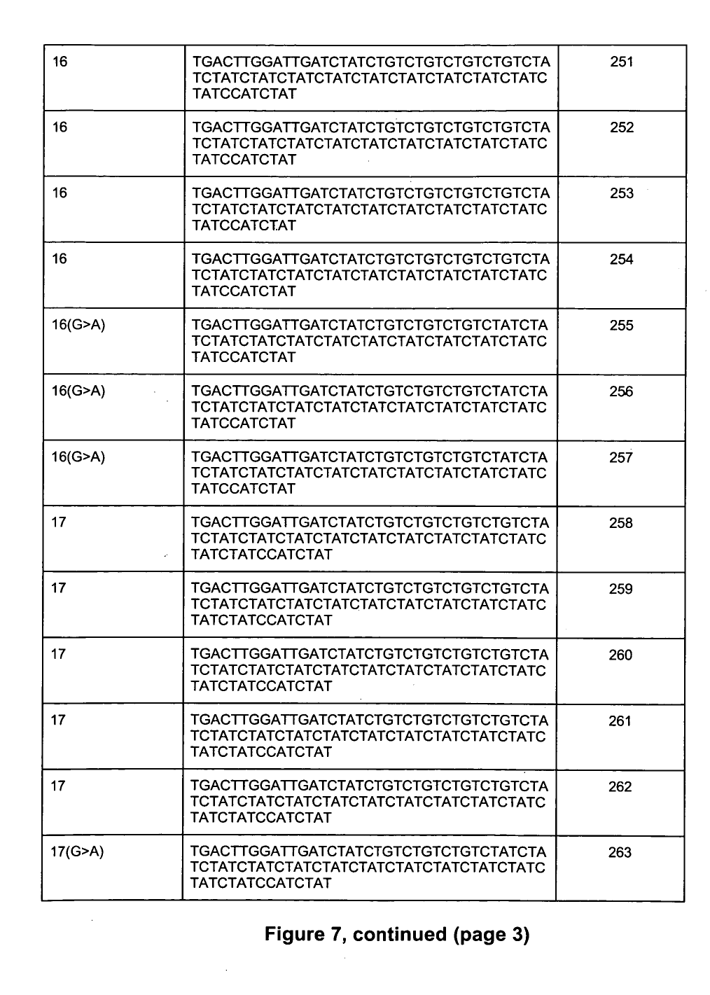

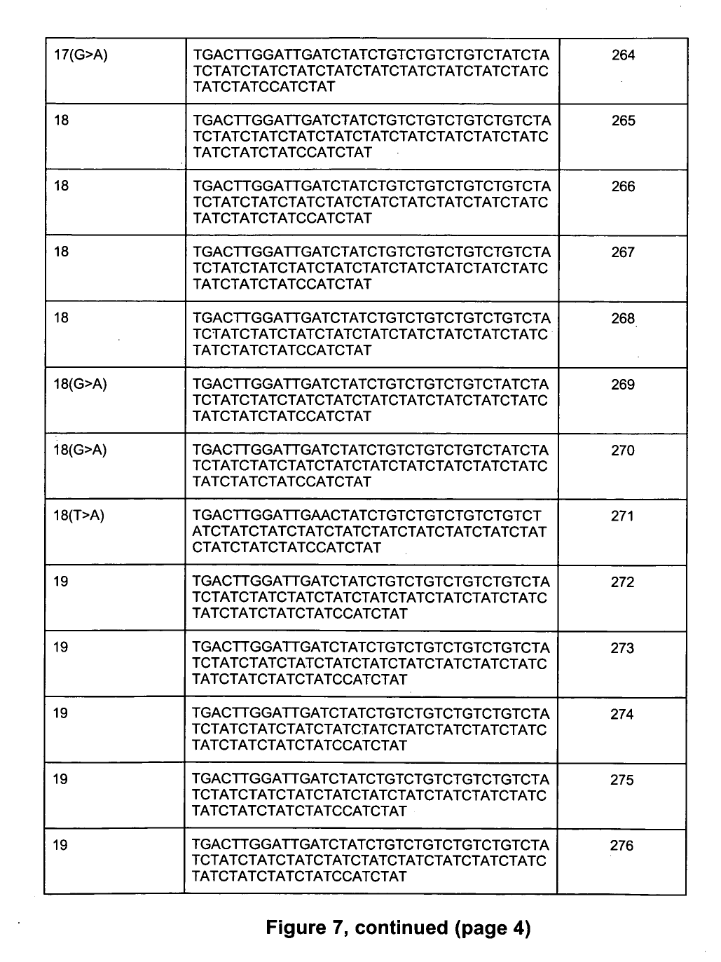

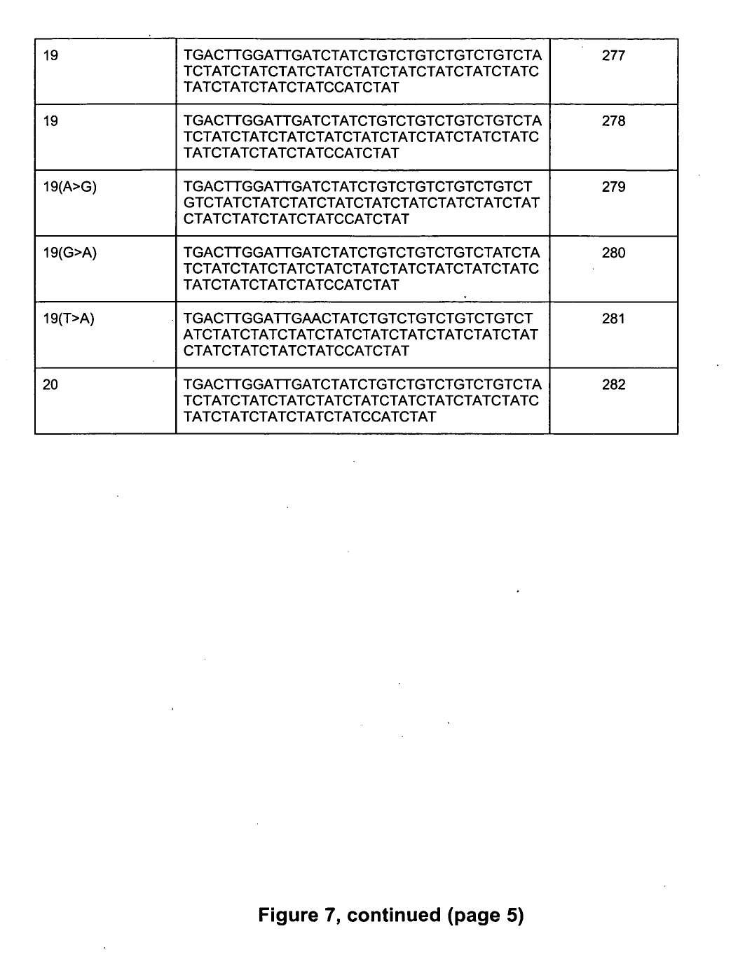

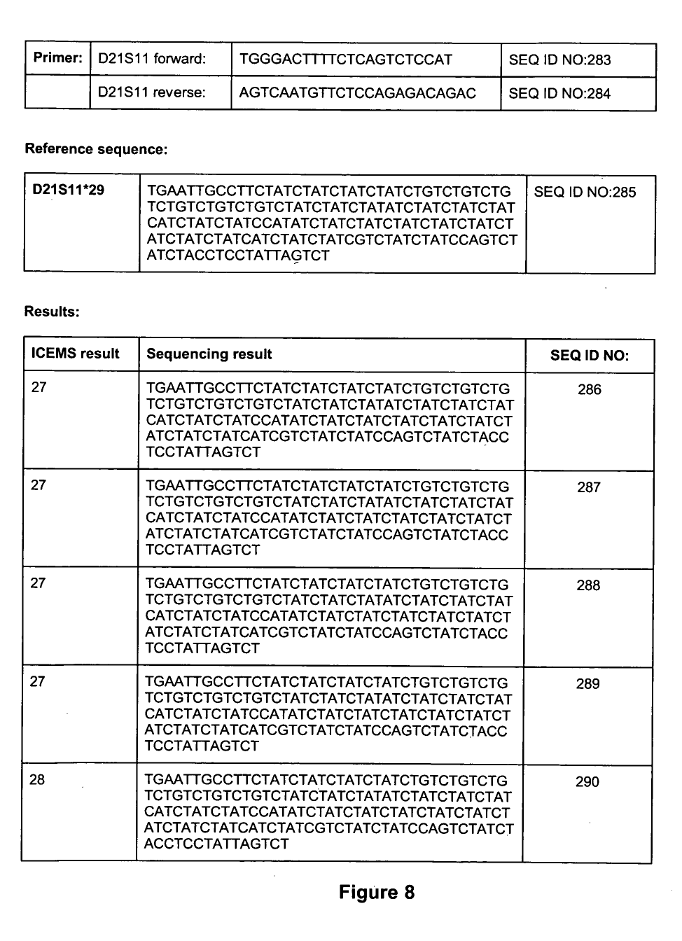

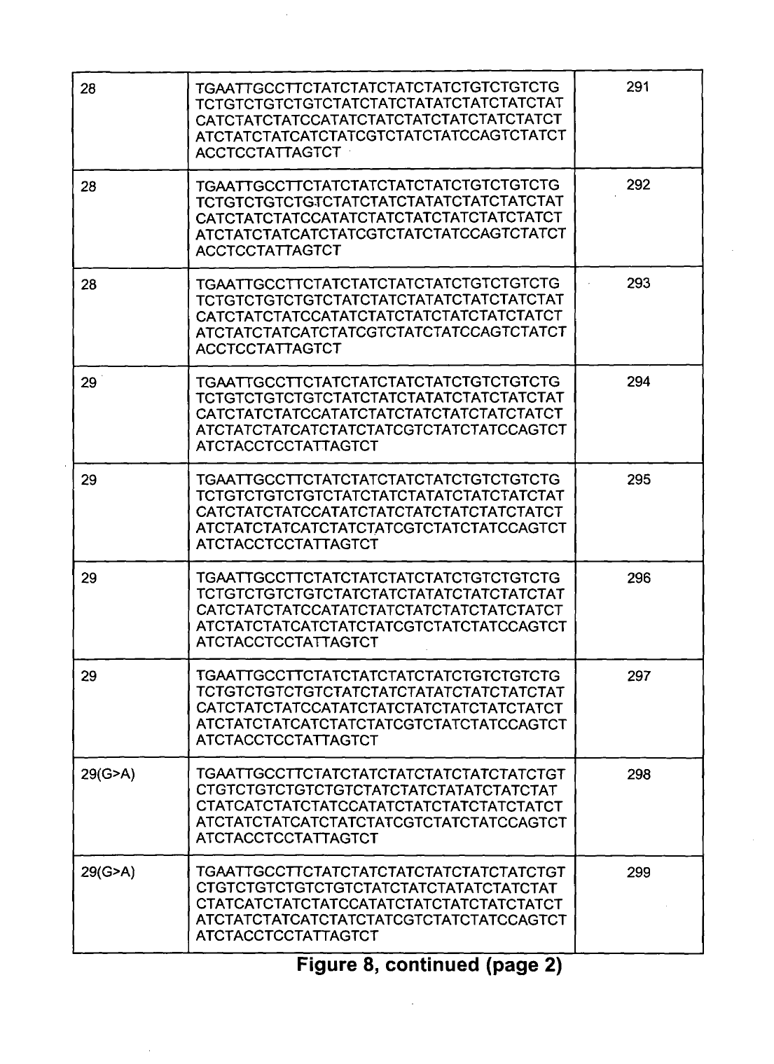

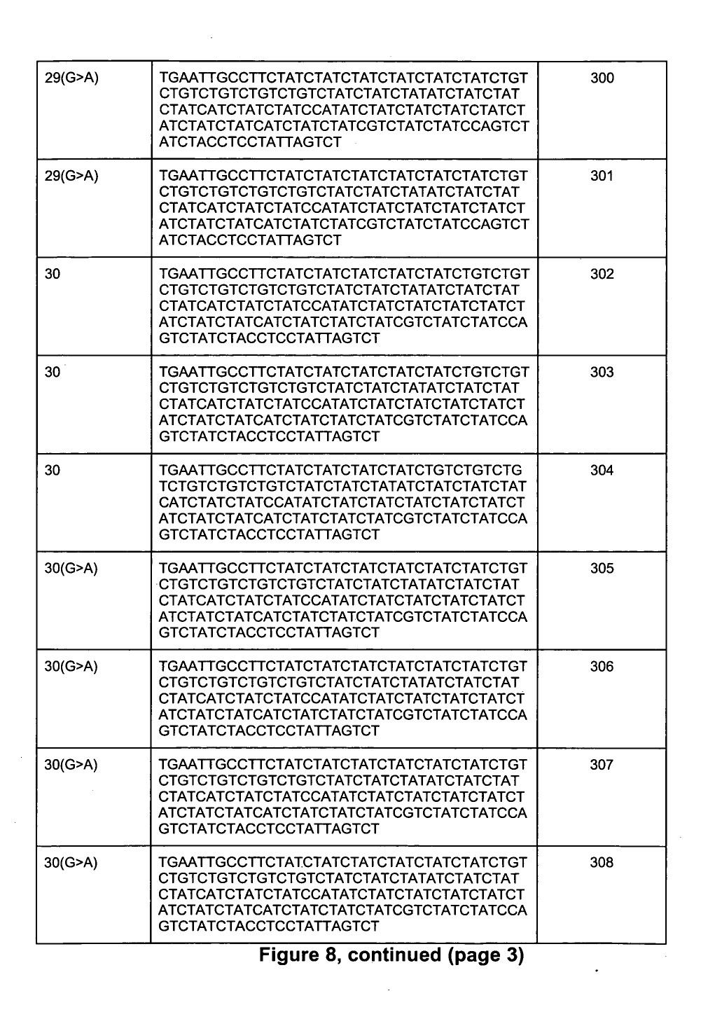

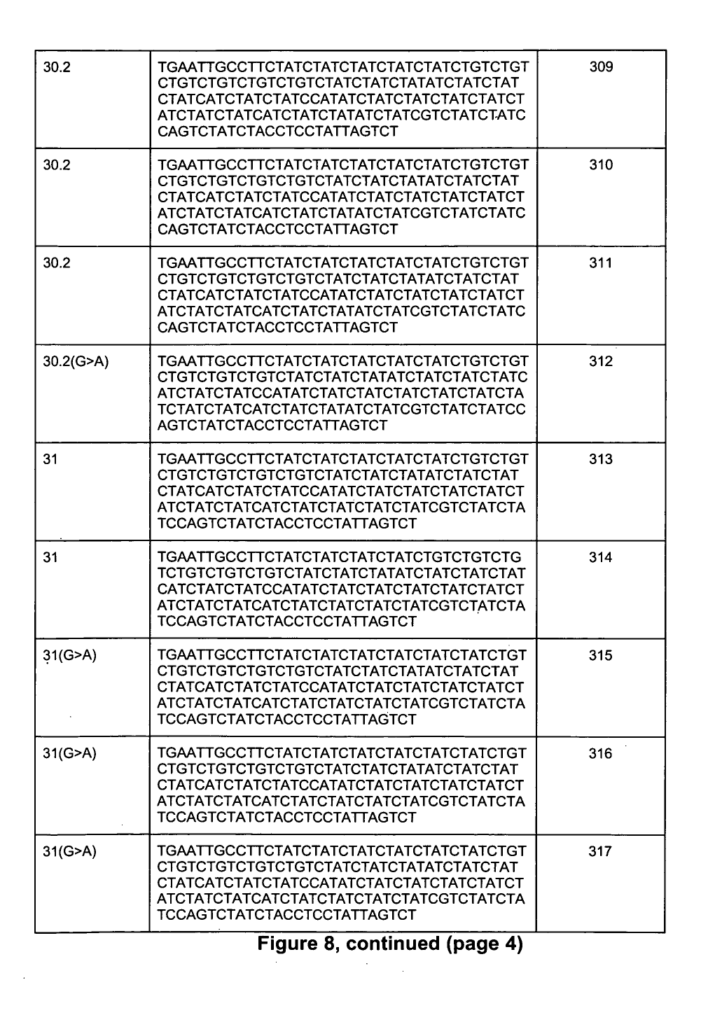

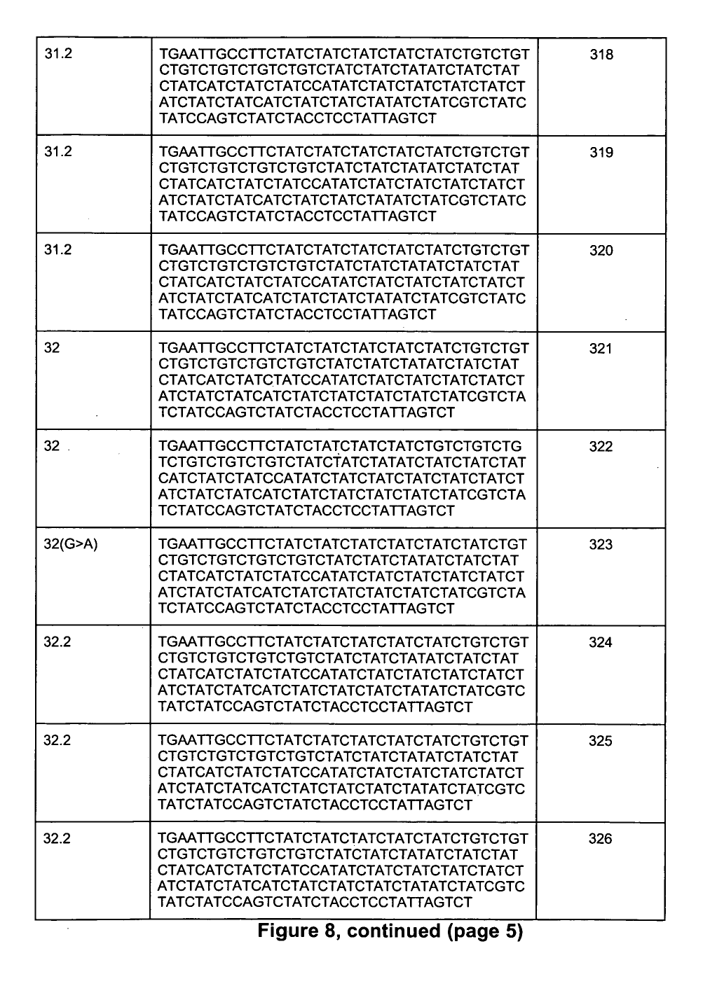

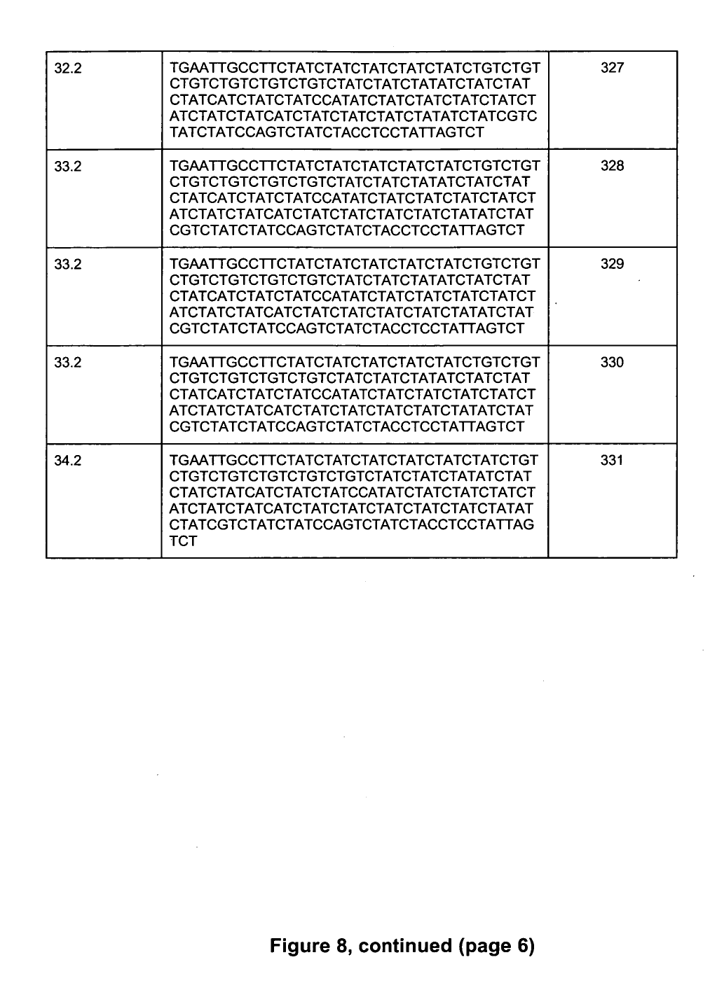

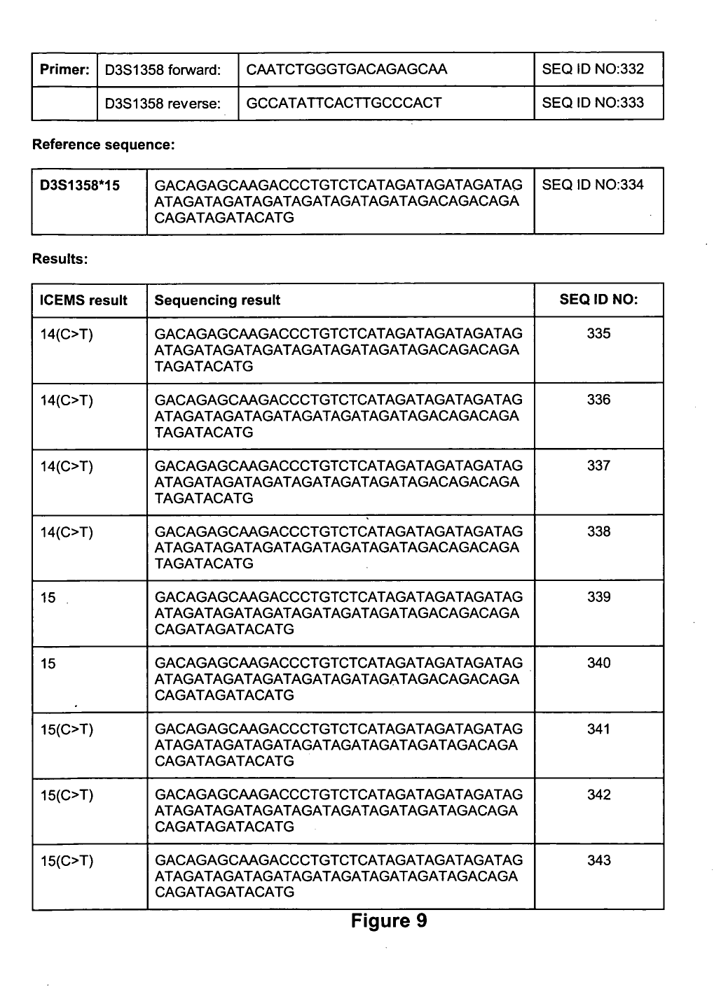

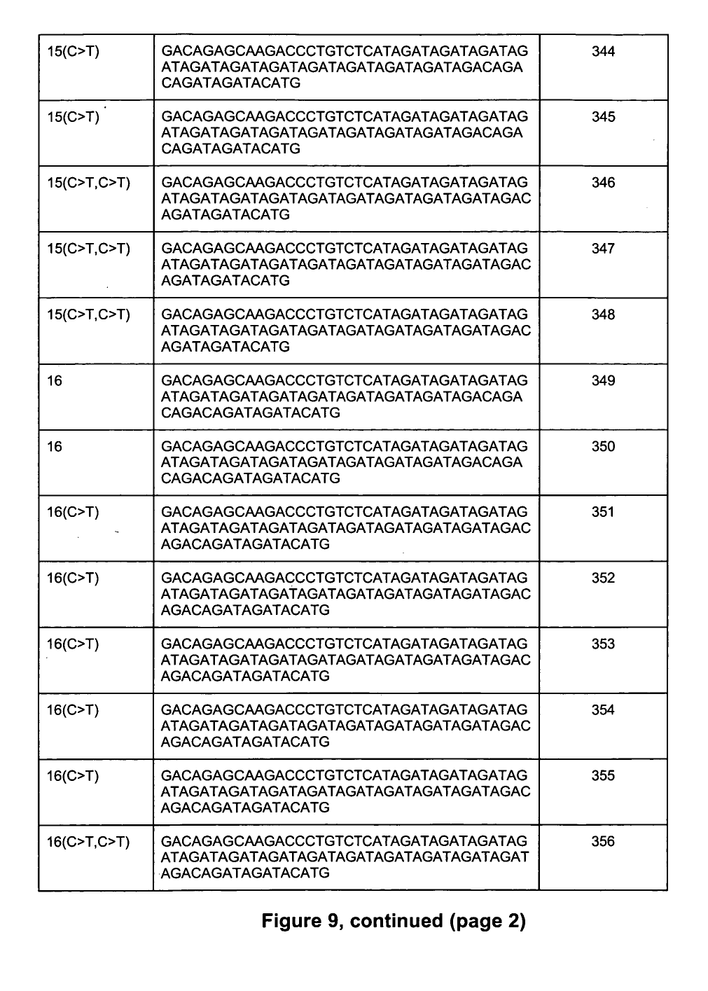

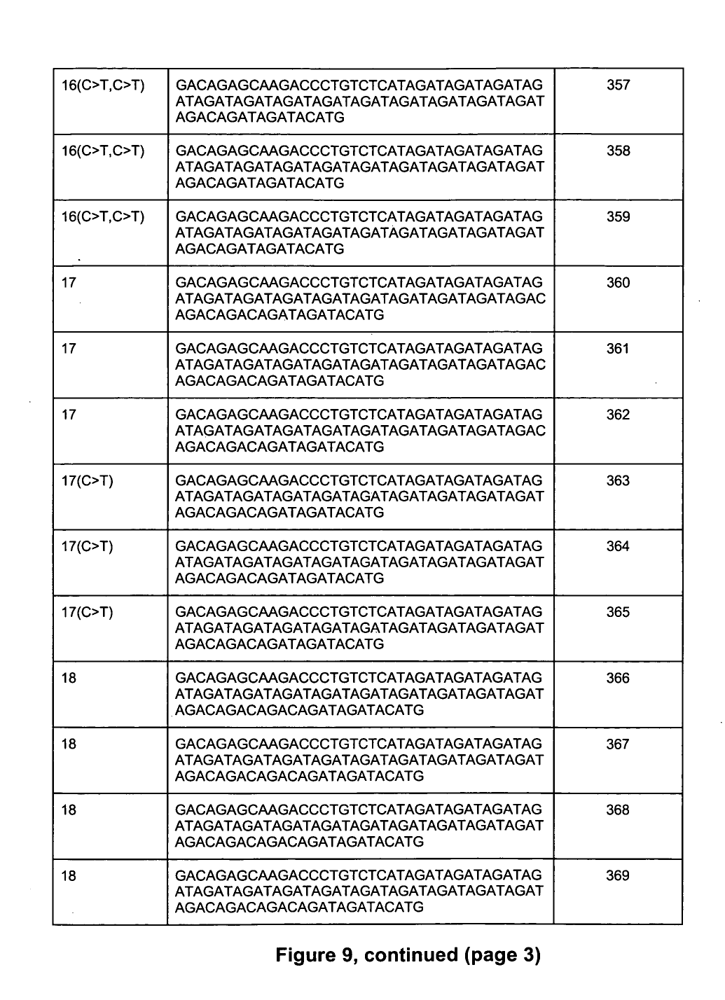

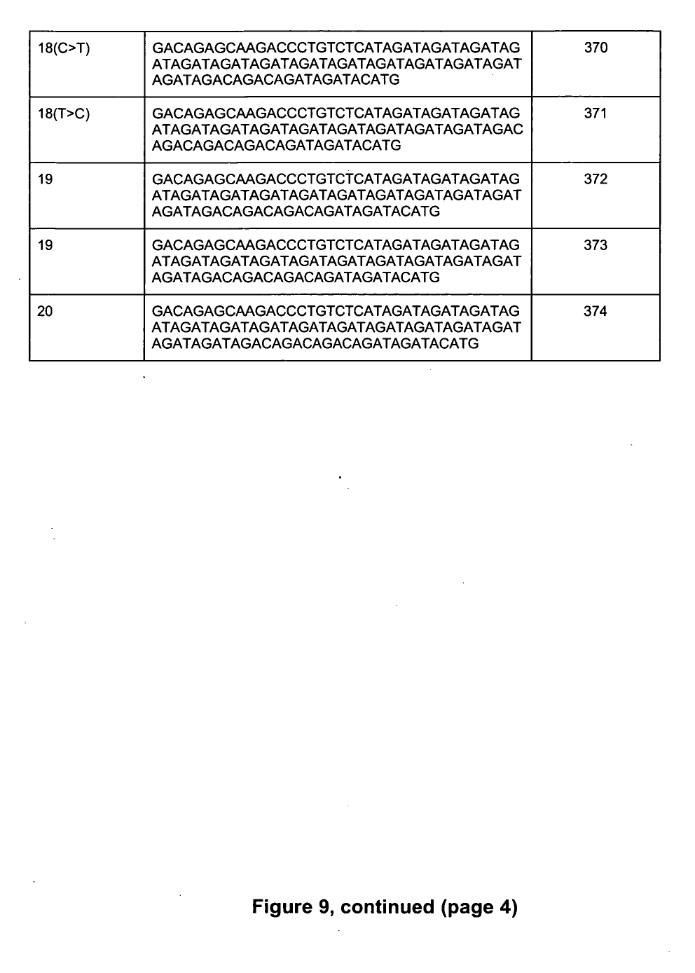

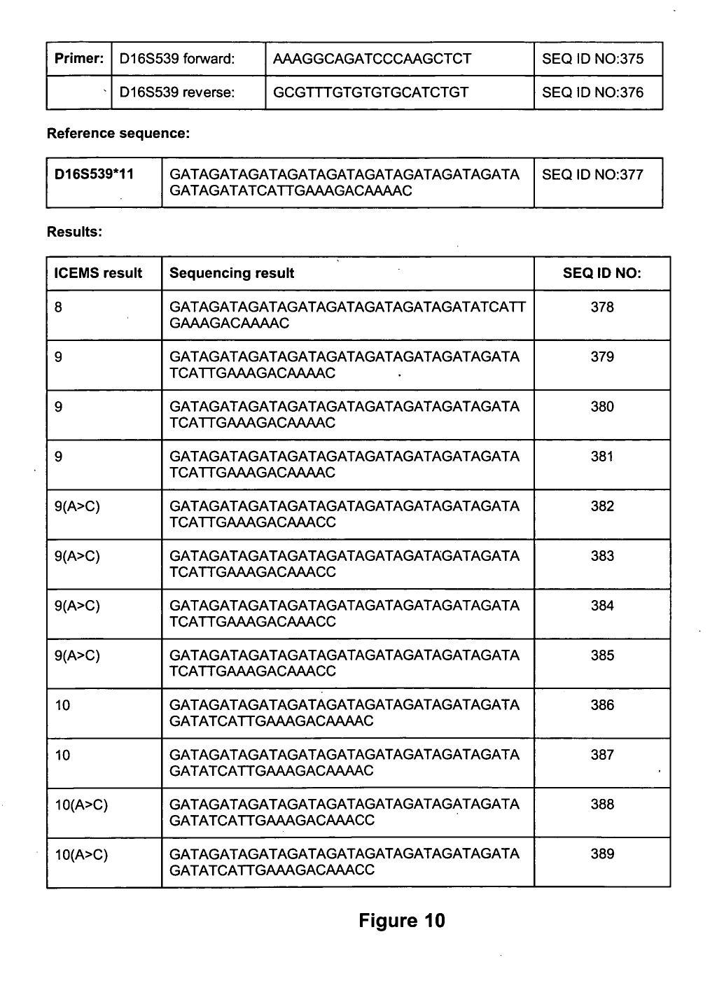

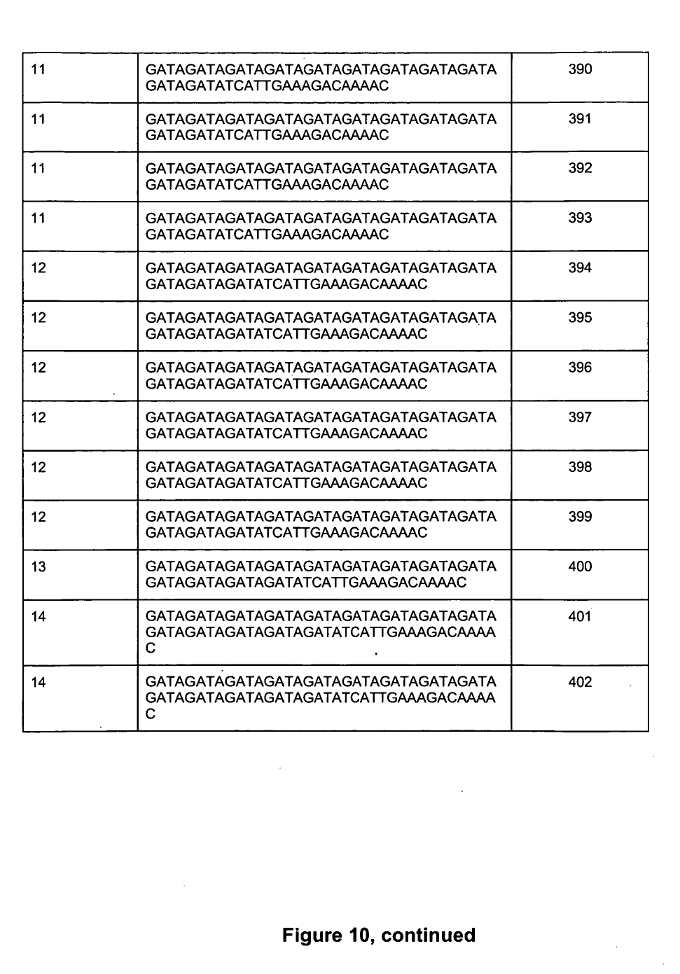

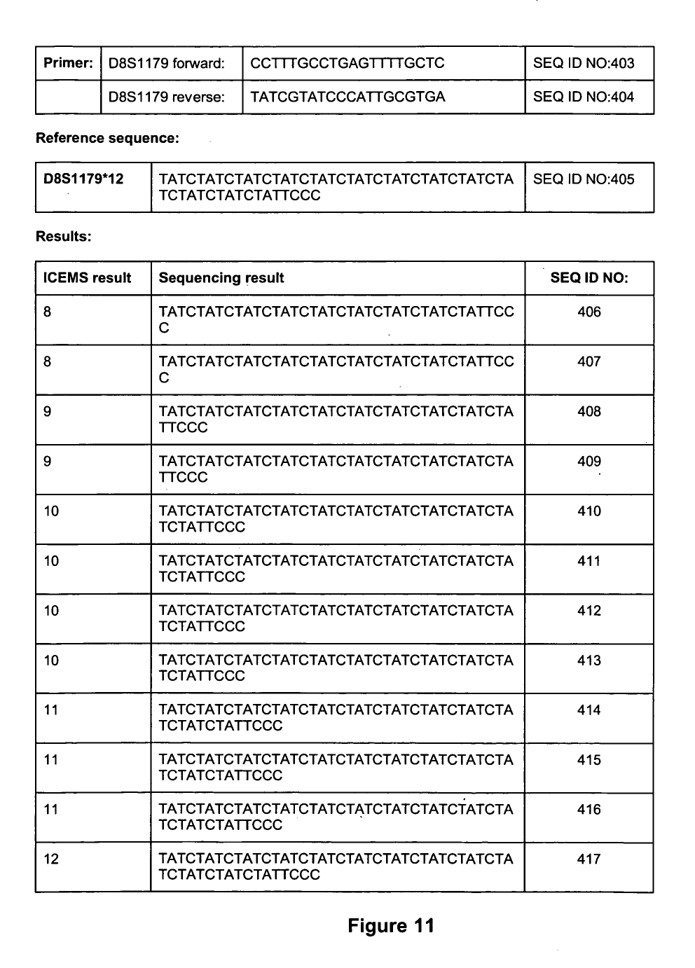

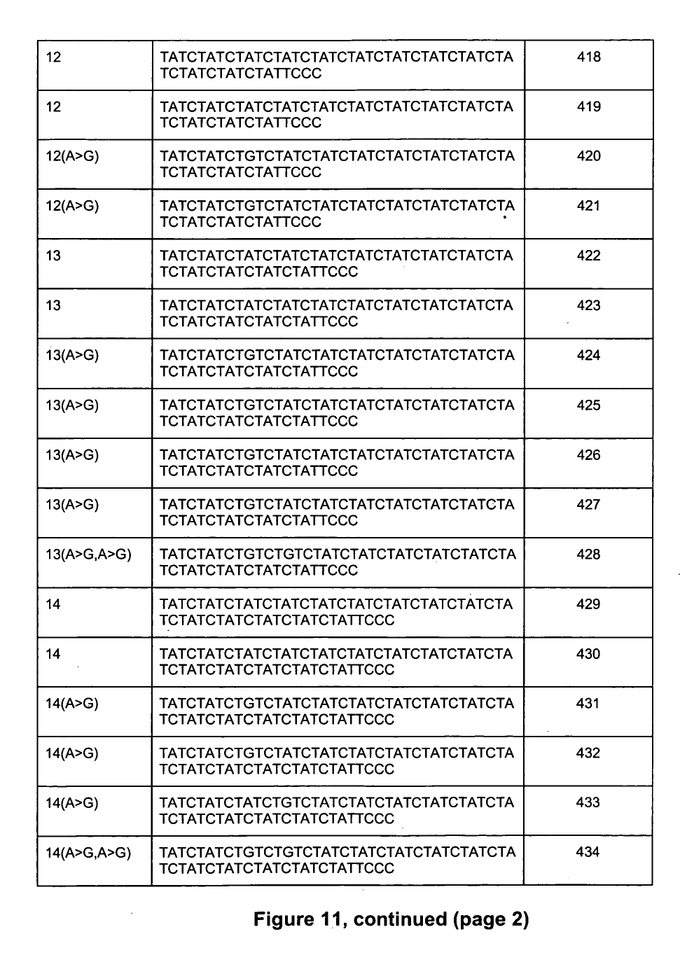

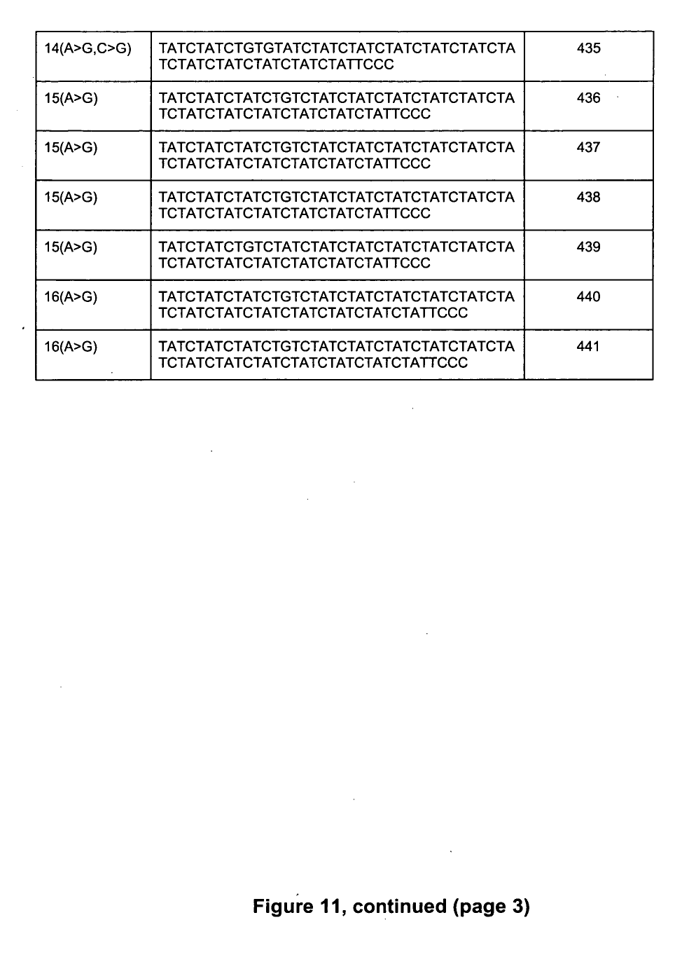

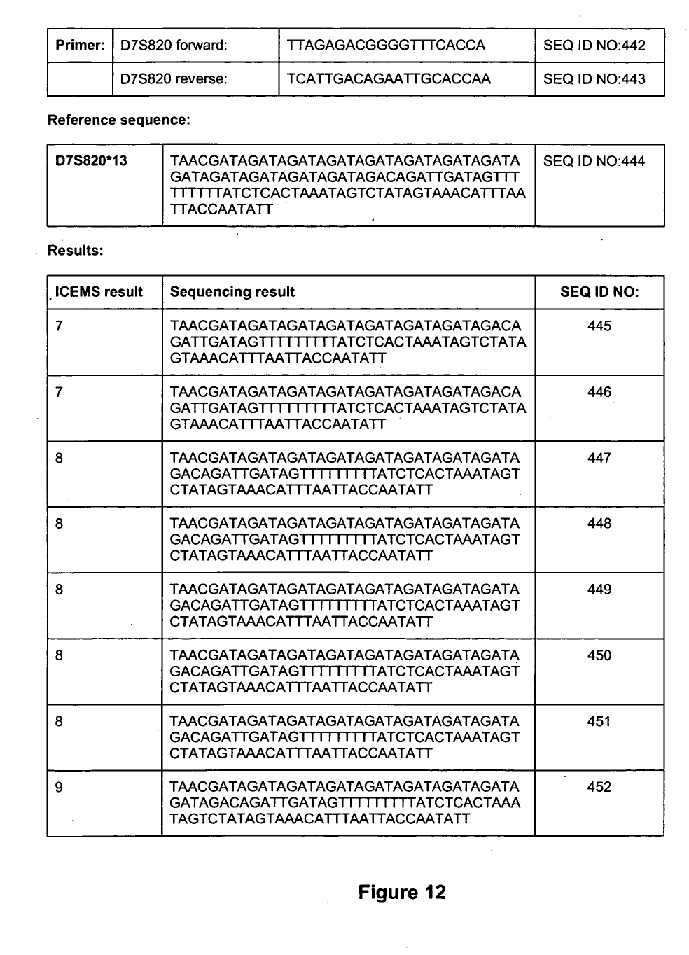

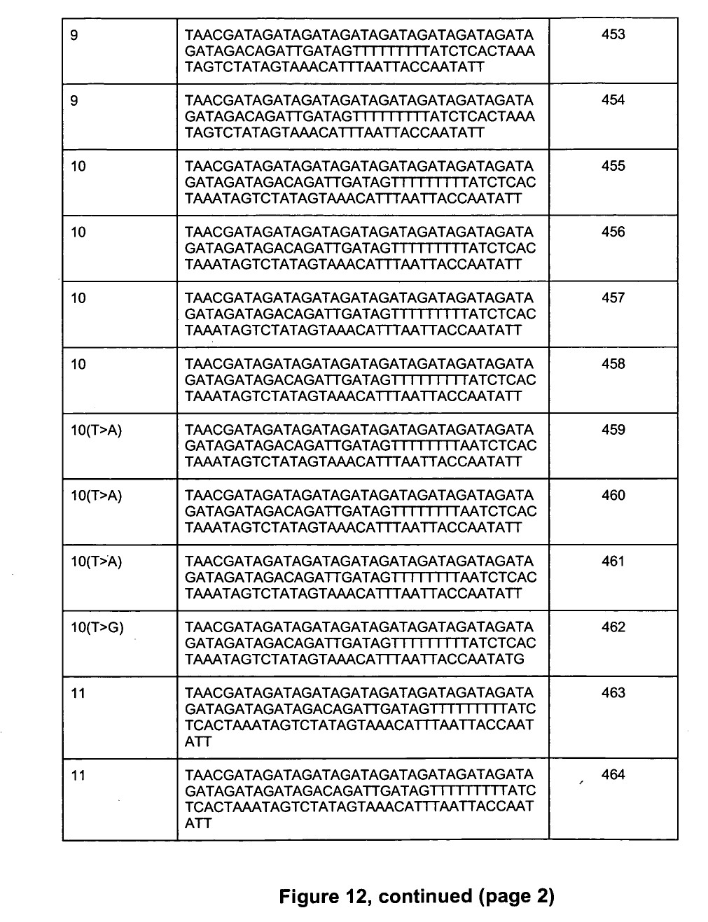

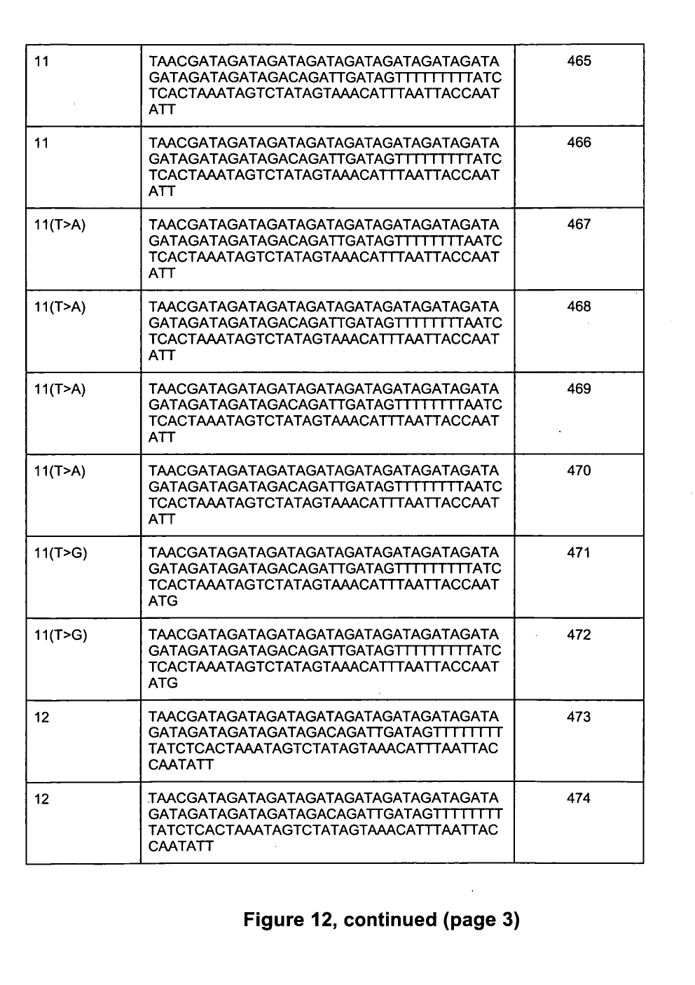

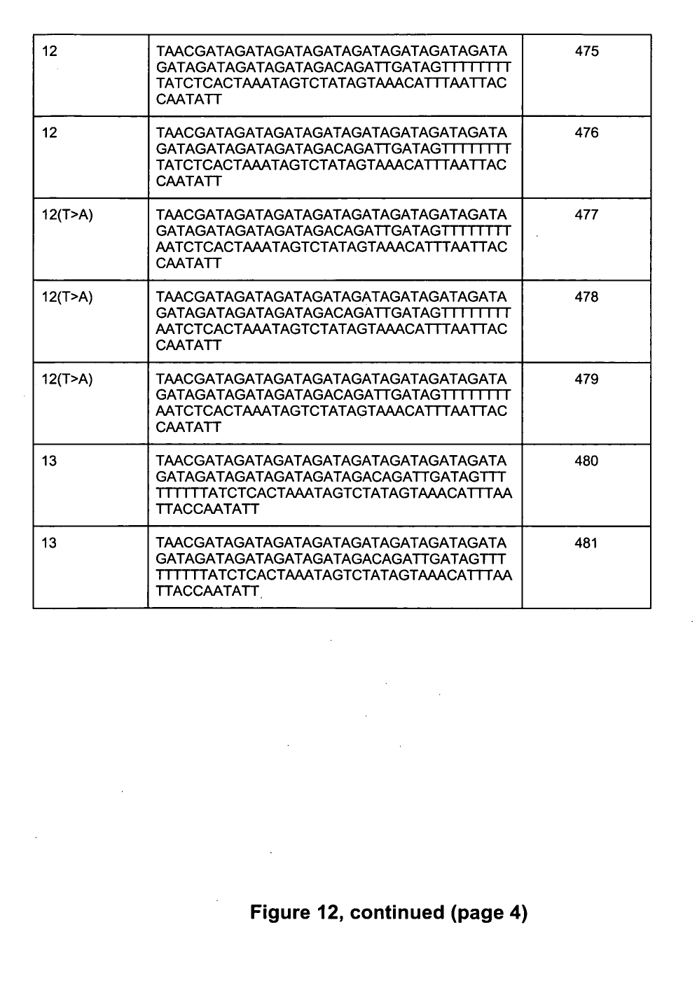

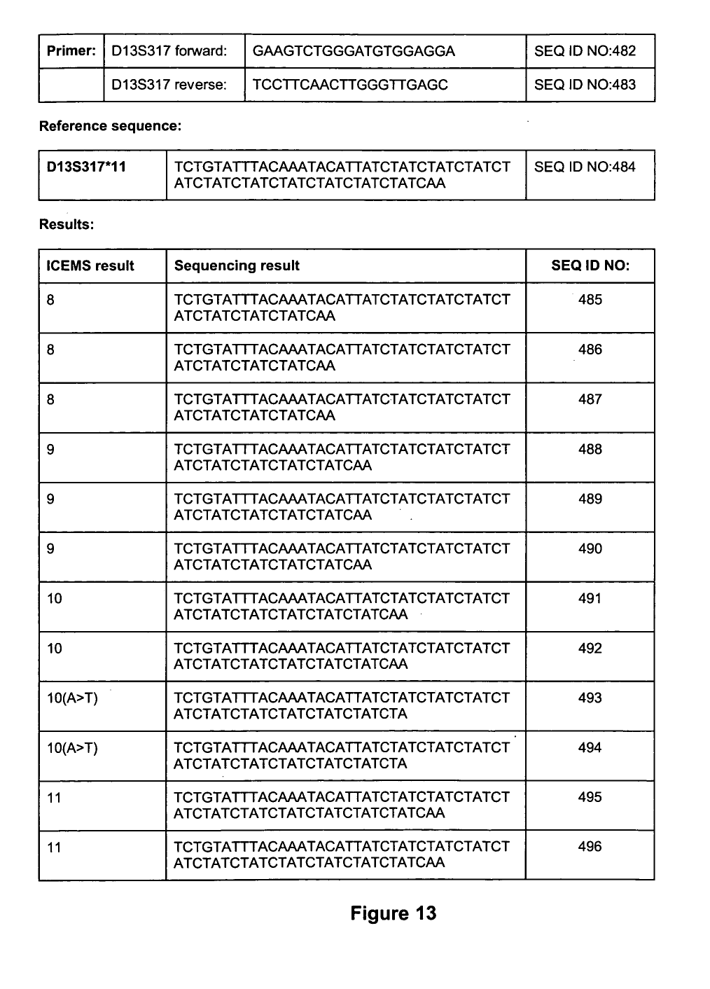

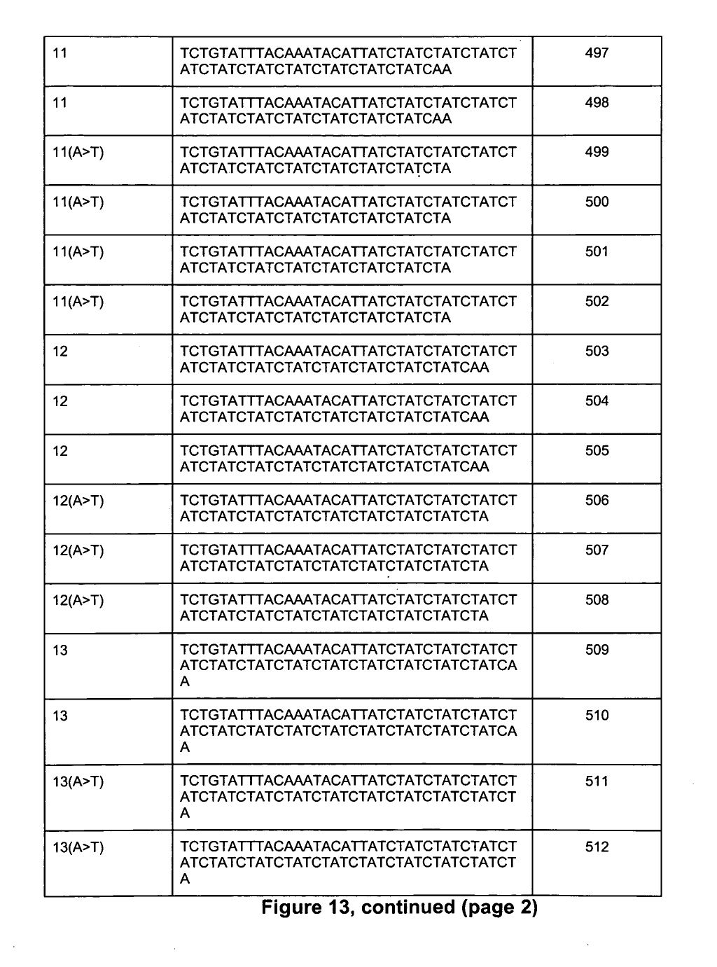

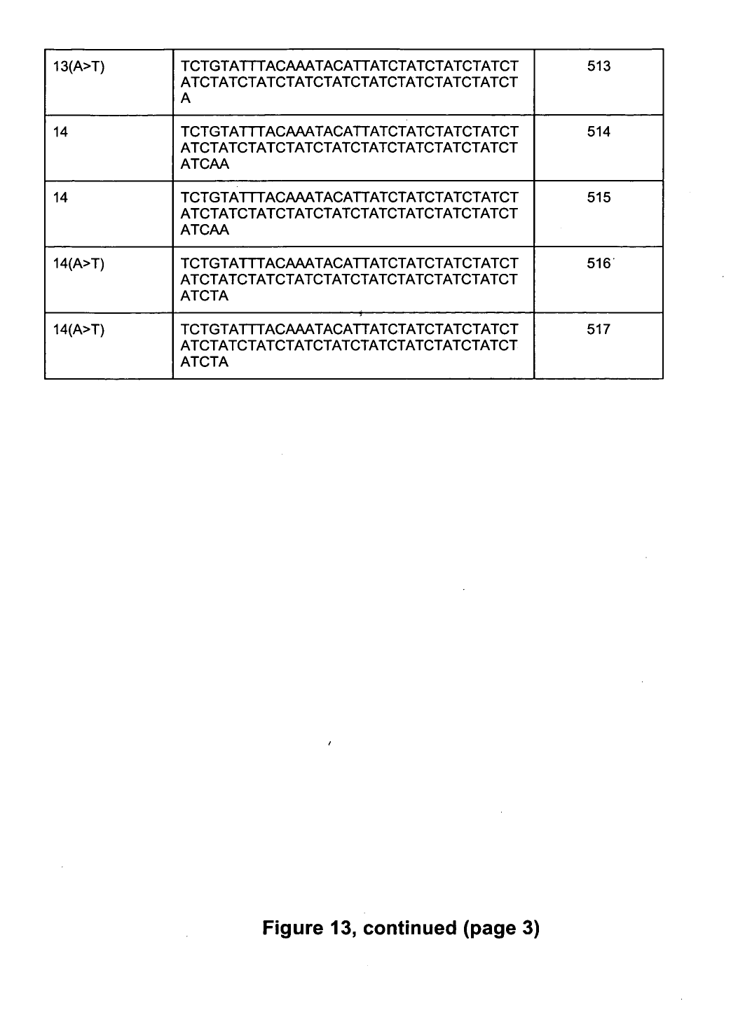

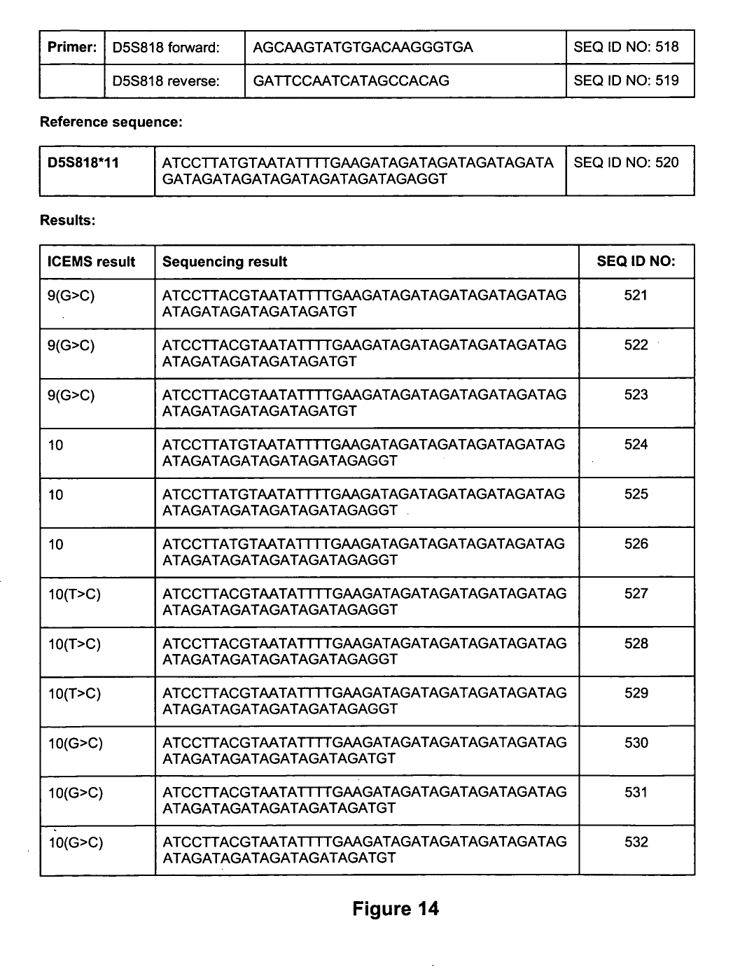

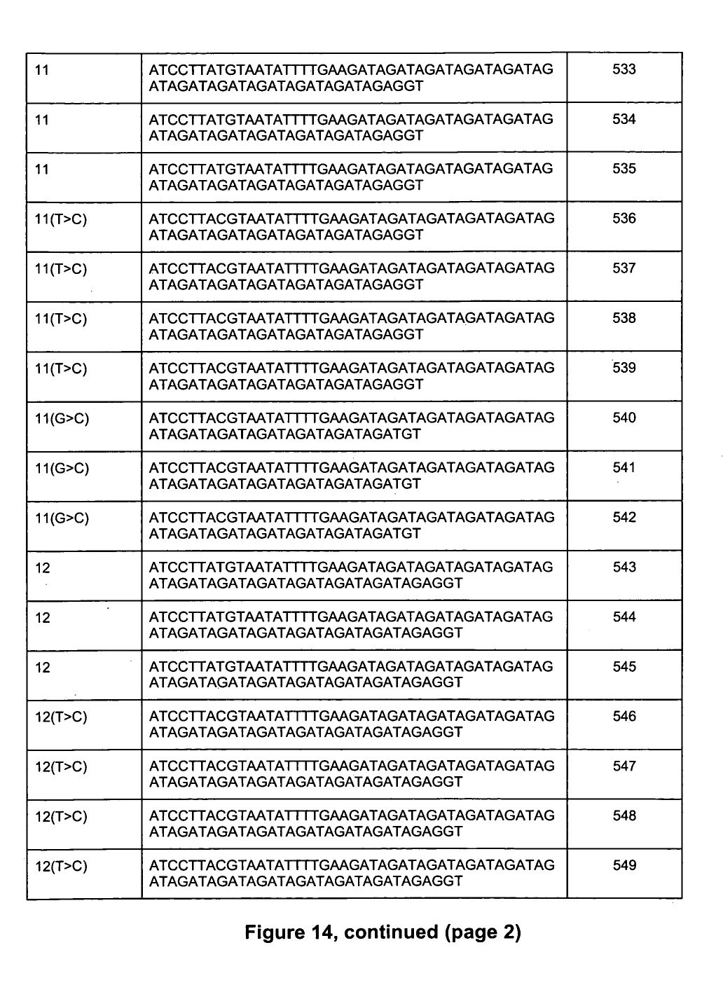

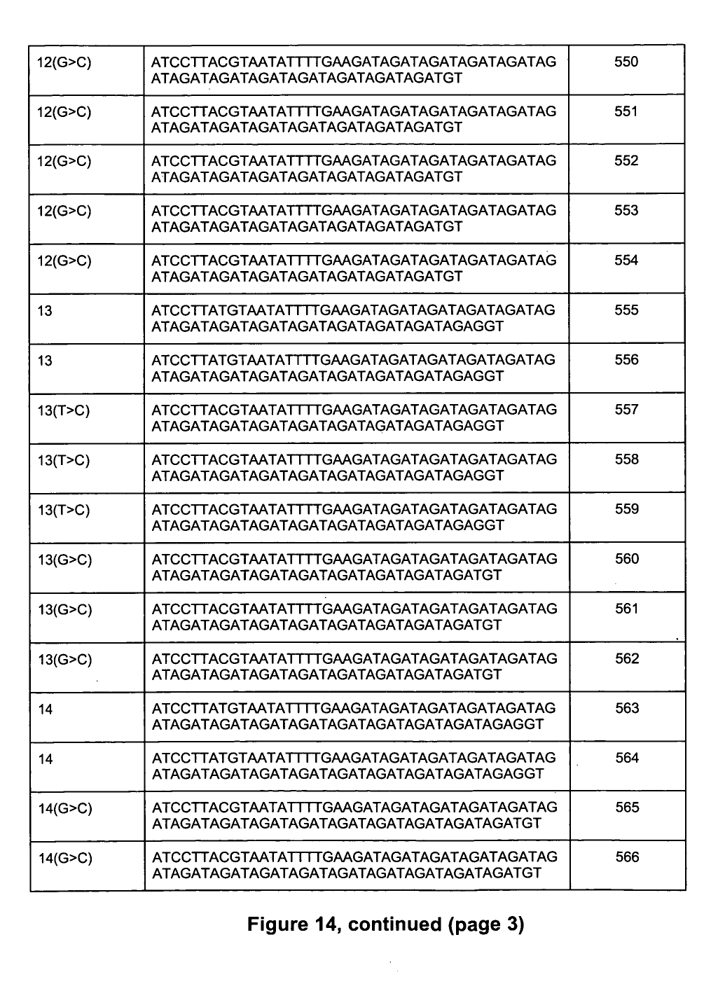

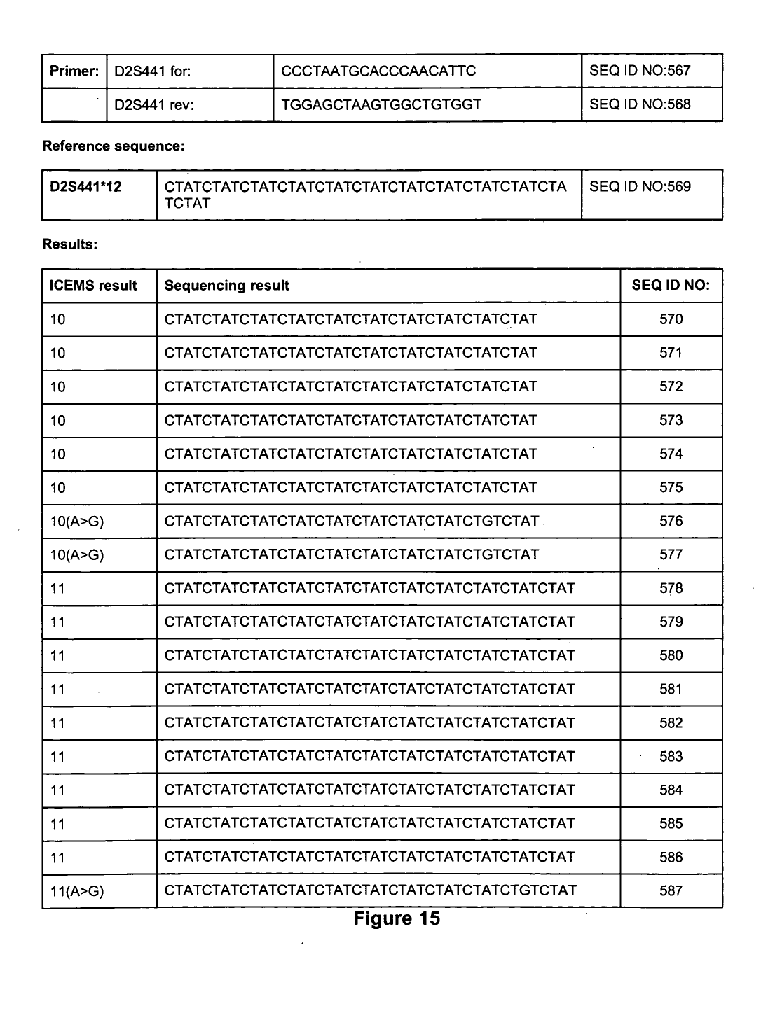

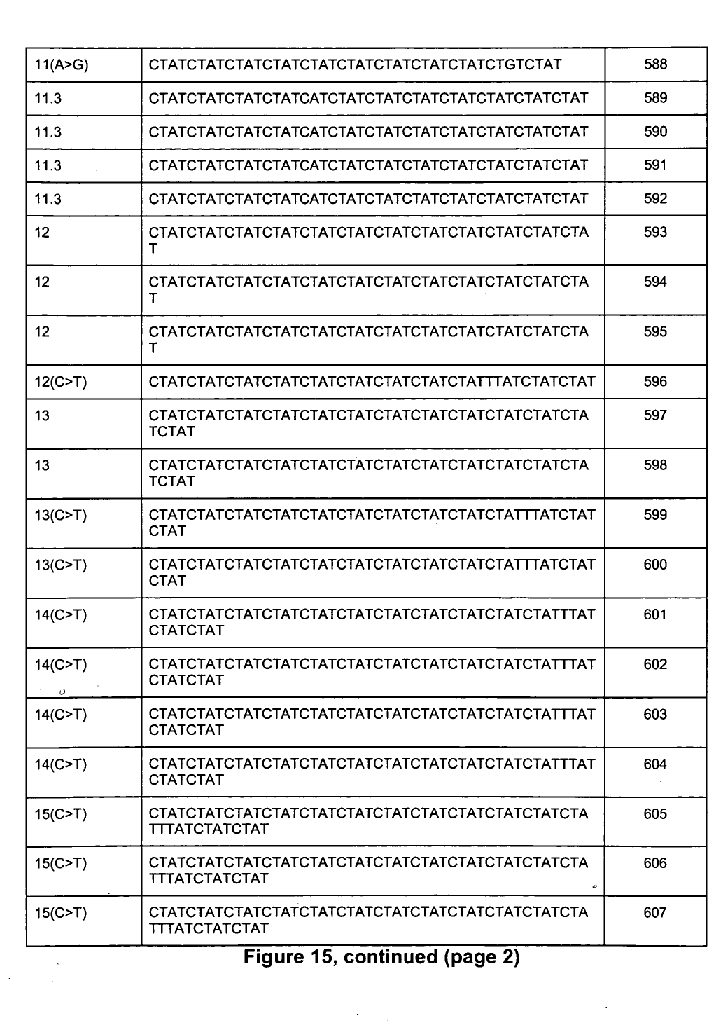

In a third set of experiments, a representative number of alleles were characterized by direct sequencing analysis using Sanger sequencing. For all STR markers, the results obtained from direct sequencing of PCR products correlated well with the ICEMS results. A summary of the sequencing results can be found in FIGS. 3-15.

### Example 1

### Evaluation of Various Embodiments of the Methods

### Instruments and Materials

To obtain samples, buccal swaps were taken from volunteers and DNA was extracted using the Chelex method (M. Steinlechner et al. (2001) Int. J. Legal Med. 114: 288-290). The primer pairs outlined in Table 3 were used for PCR amplification, which was conducted in a Gene Amp PCR System 9700 (Applied Biosystems, Foster City, Calif.) using 20 ul reactions comprising 1× AmpliTaq Gold PCR Buffer II (Applied Biosystems), 1.5 mM MgCl₂, 1 ul DNA extract, 1.0 uM of each primer, 1 unit AmpliTaq Gold Polymerase (Applied Biosystems) and 0.2 mM of each dNTP. For validation purposes some samples were reamplified with a dNTP mixture containing 0.4 mM dUTP instead of dTTP. Amplification was carried out in a Gene Amp PCR System 9700 (Applied Biosystems) starting with an initial denaturation step at 95° C. for 10 min followed by 40 cycles of 94° C. for 30 s, 52° C. (68° C. for D19S433) for 45 s, and 72° C. for 30 s, and a final extension step of 72° C. for 60 min. An Ultimate fully integrated capillary HPLC system (LCPackings, Amsterdam, The Netherlands) in combination with a Famos micro autosampler (LC-Packings) equipped with a 1 RL loop was used for all chromatographic experiments. The 50×0.2 mm i.d. monolithic capillary column was prepared according to the published protocol (A. Premstaller et al. (2000) Anal. Chem. 72: 4386-4393). The flow rate was set to 2.0 ul/min. A column temperature of 68° C. was used to denature the amplicons into the corresponding single strands, which were separated using a gradient of 2.5% to 50% acetonitrile in 25 mM cychexyldimethylammonium acetate (pH 8.4) within 7 min. The gradient was started 3 min. after the injection. Eluting nucleic acids were detected on-line by negative ESI-MS which was performed on a QSTAR XL mass spectrometer (Applied Biosystems) equipped with a modified TurbolonSpray source (H. Oberacher et al. (2005) Anal. Chem. 77: 4999-5008, H. Oberacher et al. (2005) J. Mass. Spectrom. 40: 932-945). Mass calibration and optimization of instrumental parameters was performed as described elsewhere (H. Oberacher et al. (2005) Anal. Chem. 77: 4999-5008, H. Oberacher et al. (2005) J. Mass. Spectrom. 40: 932-945). The spray voltage was set to 4.0 kV. Gas flows of 15 arbitrary units (nebulizer gas) and 45 arbitrary units (turbo gas) were employed. The temperature of the turbo gas was adjusted to 300° C. The accumulation time was set to 1 s and 10 time bins were summed up. Mass spectra were recorded in the range between 800 u and 1200 u on a personal computer operating with the Analyst QS software (service pack 8 Applied Biosystems). Deconvolution of raw mass spectra was performed with Bayesian Protein Reconstruct (BioAnalyst 1.1.1, Applied Biosystems).

TABLE 3
Primer pairs used for PCR amplification of STR loci.

| | | SEQ ID |
|---|---|---|
| STR locus*allele | PCR amplification primers | NO |
| SE33*25.2 | 5′-GAAAGAGACAAAGAGAGTTAG-3′ | 1 |
| | 5′-ACATCTCCCCTACCGCTATAG-3′ | 2 |
| D2S1338*17 | 5′-CAGTGGATTTGGAAACAGAAATG-3′ | 3 |
| | 5′-TCAGTAAGTTAAAGGATTGCAGG-3′ | 4 |
| vWA*18 | 5′-CCCTAGTGGATGATAAGAATAATCAGTATG-3′ | 5 |
| | 5′-GGACAGATGATAAATACATAGGATAGGATGGATGG-3′ | 6 |
| D21S11*29 | 5′-ATATGTGAGTCAATTCCCCAAG-3′ | 7 |
| | 5′-GGTAGATAGACTGGATAGATAGACGA-3′ | 8 |
| D3S1358*15 | 5′-ACTGCAGTCCAATCTGGGT-3′ | 9 |
| | 5′-ATGAAATCAACAGAGGCTTG-3′ | 10 |
| D16D539*11 | 5′-ATACAGACAGACAGACAGGTG-3′ | 11 |
| | 5′-GCATGTATCTATCATCCATCTCT-3′ | 12 |
| D8S1179*12 | 5′-TTTTTGTATTTCATGTGTACATTCG-3′ | 13 |
| | 5′-CGTAGCTATAATTAGTTCATTTTCA-3′ | 14 |
| D7S820*13 | 5′-GAACACTTGTCATAGTTTAGAACGAAC-3′ | 15 |
| | 5′-TCATTGACAGAATTGCACCA-3′ | 16 |
| D13S317*11 | 5′-TCTGACCCATCTAACGCCTA-3′ | 17 |
| | 5′-CAGACAGAAAGATAGATAGATGATTGA-3′ | 18 |
| D5S818*11 | 5′-GGGTGATTTTCCTCTTTGGT-3′ | 19 |
| | 5′-AACATTTGTATCTTTATCTGTATCCTTATTTAT-3′ | 20 |
| D2S441*12 | 5′-CTGTGGCTCATCTATGAAAACTT-3′ | 21 |
| | 5′-GAAGTGGCTGTGGTGTTATGAT-3′ | 22 |
| D19S433*12 | 5′-TGCACTCCAGCCTGGGCAAC-3′ | 23 |
| | 5′-TTGGTGCACCCATTACCCGAAT-3′ | 24 |
| FGA*21 | 5′-GGCATATTTACAAGCTAGTTTCT-3′ | 25 |
| | 5′-ATTTGTCTGTAATTGCCAGC-3′ | 26 |
| D18S51*18 | 5′-TGAGTGACAAATTGAGACCTT-3′ | 27 |
| | 5′-GTCTTACAATAACAGTTGCTACTATT-3′ | 28 |
| CSF1PO*13 | 5′-ACAGTAACTGCCTTCATAGATAG-3′ | 29 |
| | 5′-GTGTCAGACCCTGTTCTAAGTA-3′ | 30 |
| PentaD*13 | 5′-GAGCAAGACACCATCTCAAGAA-3′ | 31 |
| | 5′-GAAATTTTACATTTATGTTTATGATTCTCT-3′ | 32 |
| PentaE*5 | 5′-GGCGACTGAGCAAGACTC-3′ | 33 |
| | 5′-GGTTATTAATTGAGAAAACTCCTTACA-3′ | 34 |
| TH01*9 | 5′-CCTGTTCCTCCCTTATTTCCC-3′ | 35 |
| | 5′-GGGAACACAGACTCCATGGTGA-3′ | 36 |
| TPOX*8 | 5′-CTTAGGGAACCCTCACTGAATG-3′ | 37 |
| | 5′-GTCCTTGTCAGCGTTTATTTGC-3′ | 38 |
| D10S1248*13 | 5′-TTAATGAATTGAACAAATGAGTGAG-3′ | 39 |
| | 5′-TACAACTCTGGTTGTATTGTCTTCAT-3′ | 40 |
| D22S1045*17 | 5′-ATTTTCCCCGATGATAGTAGTCT-3′ | 41 |
| | 5′-GCGAATGTATGATTGGCAATATTTTT-3′ | 42 |

Sanger sequencing of a representative number of alleles was performed as described elsewhere (A. P. Hellmann et al. (2006) J. Forensic Sci. 51: 274-281). The obtained results can be found in the FIGS. 3-15 of the present application. The statistical analysis of the genotyping results was performed as described elsewhere (M. Steinlechner et al. (2001) Int. J. Legal Med. 114: 288-290).

### Discussion of Results

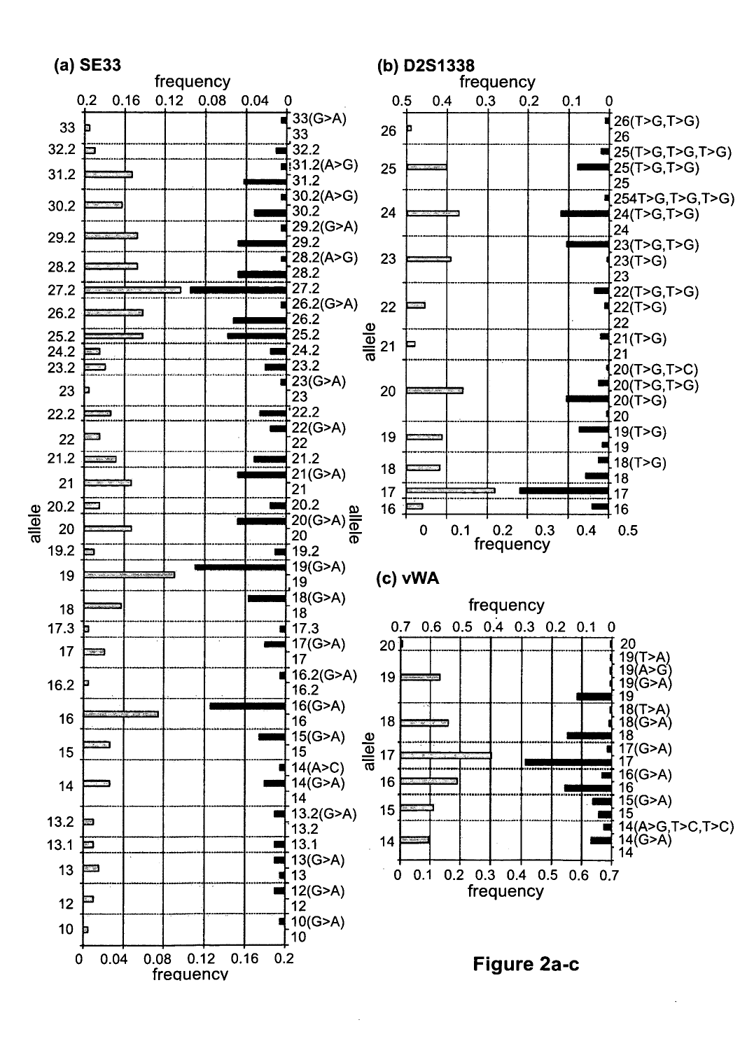

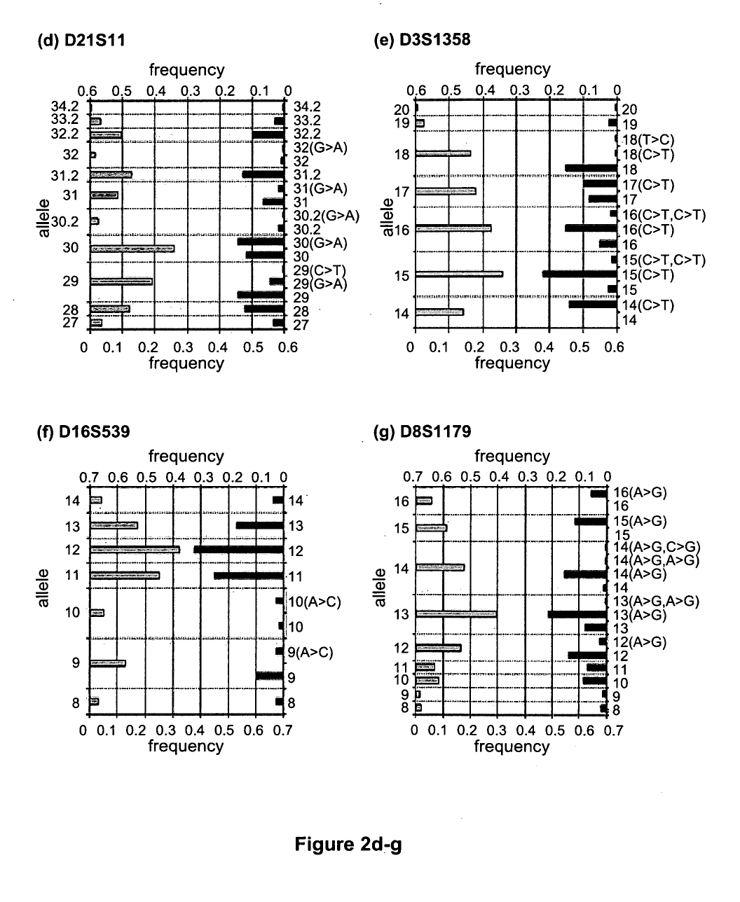

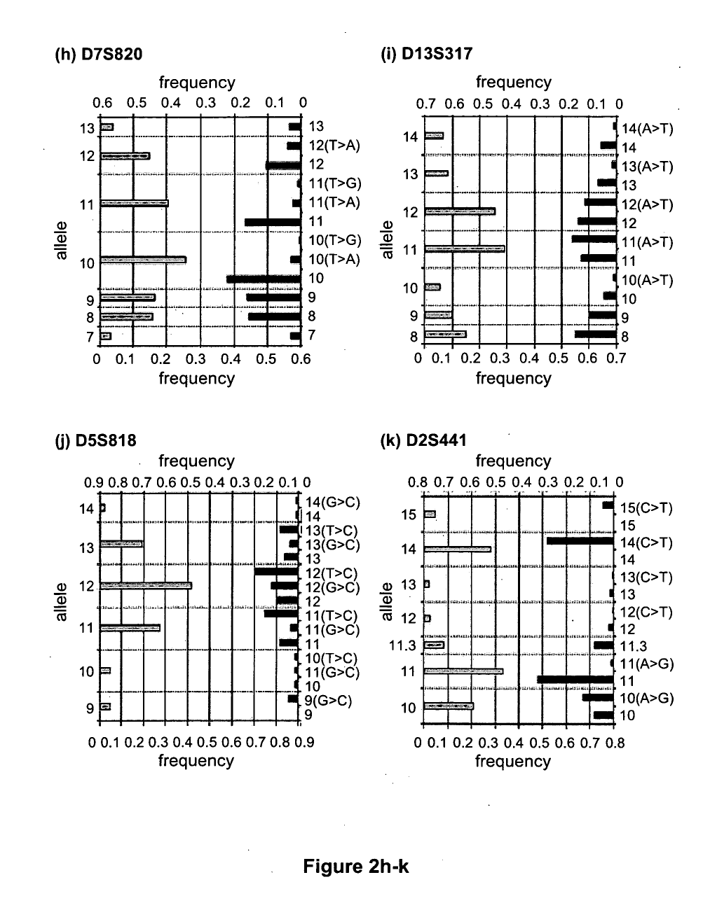

In the following section the results obtained from genotyping of the 11 STR markers showing nucleotide variability within an Austrian population sample are discussed. Referring to FIG. 2A, SE33 is a complex repeat in which 32 length variants were identified via electrophoretic sizing compared to 39 alleles that were distinguished with the methods of the present teachings (ICEMS results). Direct sequencing showed that nucleotide variations were located either within the repeat blocks or within the sequence framed by the repeat unit and the reverse primer. In the latter case, the SNP rs9362477 was responsible for the majority of detected variations.

Referring to FIG. 2B, the nucleotide variability observed for D2S1338 was related to changes within the repeat block. On one hand the “TGCC-“TTCC”-ratio was variable and on the other hand the addition of one “TCCG”-unit to alleles consisting of 20 and more repeat blocks was observed; and the number of distinguishable alleles was increased from 11 up to 20 using embodiments of the methods of the present teachings (ICEMS results).

Referring to FIG. 2C, with the exception of the 14(A>G,T>C,T>C)-allele, nucleotide variability of the vWA-marker was attributable to changes within the repeat region only. The “TCTA”-“TCTG”-ratio was variable giving rise to the detection of 16 different alleles.

Referring to FIG. 2D, variability within the “TCTA”-“TCTG”-ratio was also responsible for nucleotide variability identified for D21 S11 alleles.

Referring to FIG. 2E, the repeat region of D3S1358 alleles consists of a variable number of “CAGA”-units. Thus, 14 instead of 7 alleles became distinguishable using embodiments of the methods of the present teachings (ICEMS results).

Referring to FIG. 2F, for D16S539, the SNP rs11642858 was found to be the source of nucleotide variability. Interestingly, only the alleles #9 and #10 were seen to be linked with this SNP.

Referring to FIG. 2G, according to the reference sequence, D8S1179 only consists of “TCTA”-blocks. Within the repeat region of alleles larger than 12, however, it was observed that one or two “TCTG”-units can be present as the second or the third repeat block. Hence, using embodiments of the methods of the present teachings (ICEMS results), the number of distinguishable alleles was increased from 9 up to 15.

Referring to FIG. 2H, the length variants 10, 11, and 12 of D7S820 can be linked with the SNPs rs7786079 or rs7789995, and 12 different alleles were identified by ICEMS.

Referring to FIG. 2I, at the first nucleotide position downstream of the repeat block of D13S317 the SNP rs9546005 is located. With the exception of the alleles #8 and #9, variants were detected for all alleles that arose from the presence of the SNP. Hence, five additional alleles were identified using embodiments of the methods of the present teachings (ICEMS results).

Referring to FIG. 2J, the SNP rs25768 is located in close vicinity to D5S818 and for all length variants alleles containing this SNP were identified. The group of alleles containing the SNP rs25768 was subdivided due to the presence or absence of a second SNP that was located at the fourth nucleotide position downstream of the repeat region. So the overall number of distinguishable alleles was increased from 6 up to 15 using embodiments of the methods of the present teachings (ICEMS results).

Referring to FIG. 2K, according to the reference sequence, the repeat block of D2S441 solely consists of “CTAT”-blocks. Nevertheless, it was observed that within a certain number of alleles consisting of 10 or 11 repeat units, the penultimate repeat block changed its composition to “CTGT”. Likewise, within a certain number of alleles consisting of 12, 13, 14, or 15 repeat units, the last but two repeat blocks was exchanged by “TTAT”. Thus, 11 different alleles were identified using embodiments of the methods of the present teachings (ICEMS results).

The observed allelic frequencies were used to check all markers for significant deviations from the Hardy-Weinberg expectations. Only the locus D16S539 showed a departure from Hardy-Weinberg expectation. In the following section we further compare the results obtained by the two typing platforms, CE and ICEMS, with respect to their efficiency for forensic testing (D. J. Balding et al. (1995) Proc. Natl. Acad. Sci. USA 92: 11741-11745; L. A. Foreman et al. (2001) Int. J Legal Med. 114: 147-155).

Further Comparison of CE with ICEMS

The probability of match (PM) represents one important statistical parameter which describes the number of individuals that need to be investigated in order to find the same DNA pattern again in a randomly selected individual. The frequencies of the observed genotypes are used to calculate the marker-specific PM. In Table 4, the PM values of all 11 STR markers showing length and nucleotide variability are summarized.

TABLE 4
Statistical analysis of data for STRs showing
sequence variability.

| Locus | Source of variability | N | PM | h | PE |
|---|---|---|---|---|---|
| SE33 | Length | 94 | 0.014 | 0.968 | 0.897 |
| | Length and nucleotide | 94 | 0.013 | 0.968 | 0.898 |
| D2S1338 | Length | 95 | 0.044 | 0.916 | 0.743 |
| | Length and nucleotide | 95 | 0.033 | 0.947 | 0.788 |
| vWA | Length | 99 | 0.069 | 0.788 | 0.620 |
| | Length and nucleotide | 99 | 0.048 | 0.848 | 0.698 |
| D21S11 | Length | 98 | 0.048 | 0.857 | 0.691 |
| | Length and nucleotide | 98 | 0.029 | 0.929 | 0.787 |
| D3S1358 | Length | 98 | 0.081 | 0.847 | 0.605 |
| | Length and nucleotide | 98 | 0.043 | 0.857 | 0.728 |
| D16S539 | Length | 99 | 0.105 | 0.889 | 0.576 |
| | Length and nucleotide | 99 | 0.096 | 0.889 | 0.594 |
| D8S1179 | Length | 96 | 0.061 | 0.865 | 0.657 |
| | Length and nucleotide | 96 | 0.038 | 0.906 | 0.749 |
| D7S820 | Length | 95 | 0.063 | 0.800 | 0.632 |
| | Length and nucleotide | 95 | 0.046 | 0.874 | 0.709 |
| D13S317 | Length | 92 | 0.068 | 0.717 | 0.616 |
| | Length and nucleotide | 92 | 0.034 | 0.837 | 0.759 |
| D5S818 | Length | 98 | 0.141 | 0.704 | 0.464 |
| | Length and nucleotide | 98 | 0.032 | 0.867 | 0.774 |
| D2S441 | Length | 98 | 0.114 | 0.806 | 0.532 |
| | Length and nucleotide | 98 | 0.088 | 0.837 | 0.588 |

N, number of individuals;
PM, probability of match;
h, frequency of heterozygous samples,
PE, average probability of exclusion.

In the present Examples, compared to electrophoretic sizing, ICEMS was able to resolve a larger number of different alleles and genotypes. Accordingly, the PM-values decreased significantly for most of the markers (e.g. D5S818: 0.141 vs. 0.032). Likewise, the combined PM decreased from 7.43×10⁻¹⁴ to 4.04×10⁻¹⁶. The maximum frequency of a combination of 11 genotypes was calculated to be one in 13 billions considering length variability only and one in 572 billions considering length and nucleotide variability, which roughly equals an expansion of 2-3 loci measuring length variability only. The characterization of length variability had also a major impact on the frequency of heterozygous samples (h). For the majority of markers, h was increased. With the exception of SE33 and D16S539, the embodiments of the methods of the present teachings used in these examples resolved alleles that would have otherwise been classified as homozygous (Table 4).

The average probability of exclusion (PE) represents another parameter to characterize the efficiency of STR markers for forensic testing. PE is defined as the fraction of individuals with a DNA profile different from that of a randomly selected individual. The value for each individual case will vary. The PE for a given locus, however, can be calculated from the observed allelic frequencies. As a consequence of the increased number of observed alleles using embodiments of the methods of the present teachings, marker-specific PE-values were increased (Table 4, e.g., D5S8I 8: 0.464 vs. 0.774). Likewise, the combined PE increased from 0.99999373 to 0.99999975. In all, the simultaneous analysis of length and nucleotide variability significantly enhanced the forensic efficiency of the STRs. The combined PE for a set of 11 loci analyzed by ICEMS in the present Examples would equal that of a set of 13-14 markers analyzed with CE.

The present Examples screened twenty-one STR loci that are commonly used for genetic fingerprinting using embodiments of the methods of the present teachings for the occurrence of nucleotide variability to supplement the already established length variability. In 11 (SE33, D2S1338, vWA, D21S11, D3S1358, D16S539, D8S1179, D7S820, D13S317, D5S818, D2S441) out of twenty-one STR markers, nucleotide variability was detected. Statistical evaluation of the typing results obtained from an Austrian population sample revealed that the characterization of the nucleotide variants would facilitate significantly enhance forensic efficiency. The additional information that was obtained by determining the sequence variability through embodiments of the methods of the present teachings in the present Examples equaled that of 20-30% additional loci (2-3 in a set of 11 loci) investigated for length variation only.

In 4 out of the 11 STR markers displaying sequence variability (SE33, D2S1338, D21 S11, D8S1179) Sanger sequencing offered a somewhat increased resolution in comparison to ICEMS typing. The combination of increased discrimination efficiency and of the use of small amplicons for PCR can make various embodiments of the present teachings very attractive for the typing of forensic casework samples, especially for degraded DNA. In various embodiments of the present teachings, ICEMS could be a valuable tool for kinship testing where usually a large amount of genetic information is necessary to unequivocally determine the degree of relatedness of two individuals (B. S. Weir et al. (2006) Nat. Rev. Genet. 7: 771-780). The present inventors believe that various embodiments of the present teachings represent a forward-looking alternative to electrophoretic sizing, for example: (1) various embodiments of the present teachings can compete regarding analysis time and costs with electrophoretic STR typing; (2) due to the identification of nucleotide variability, various embodiments of the present teachings surpass electrophoretic STR typing regarding its information content; and/or (3) STR results generated by various embodiments of the present teachings are readily comparable to data that are produced with conventional STR-typing. Thus, profiles generated by various embodiments of the present teachings can be matched to conventional STR-profiles stored in already existing DNA intelligence databases.

### Example 2

### Multiplex

In various embodiments, to increase the sample throughput and to facilitate reducing the amount of starting material necessary to generate a genetic fingerprint, STR loci are coamplified within a single PCR. For example, in various embodiments, the multiplexes comprise of 9-15 STRs.

Experiments, instruments and methods substantially similar to those of Example 1 were also conducted using 8-plex and 14-plex PCR. Tables 5-9 summarize the data of these multiple experiments.

Tables 5A and 5B compare CE and ICEMS genotyping data for all 21 markers, for two different samples. With the exception of D19S433, ICEMS results were consistent with the CE results regarding the length information. In various embodiments, the present methods can characterize nucleotide variability that remains unexplored with CE. For example, for sample 007, ICEMS in this example identified the presence of two different alleles at D13S317, which were unresolved by CE typing.

TABLE 5
Comparison of STR genotypes obtained by electrophoretic sizing and
ICEMS. Base changes in brackets determined by measured molecular masses and
further confirmed by direct sequence analysis.

| electrophoretic sizing | | | ICEMS* | | |
|---|---|---|---|---|---|
| marker | allele 1 | allele 2 | marker | allele 1 | allele 2 |

5A. Sample 007

| | | | | | |
|---|---|---|---|---|---|
| SE33 | 17 | 25.2 | SE33 | 17(G > A) | 25.2 |
| D2S1338 | 20 | 23 | D2S1338 | 20(T > G) | 23(T > G, T > G) |
| vWA | 14 | 16 | vWA | 14(A > G, T > C, T > | 16 |
| | | | | C) | |
| D21S11 | 28 | 31 | D21S11 | 28 | 31(G > A) |
| D3S1358 | 15 | 18 | D3S1358 | 15(C > T) | 18(C > T) |
| D16S539 | 9 | 10 | D16S539 | 9 | 10(A > C) |
| D8S1179 | 12 | 13 | D8S1179 | 12 | 13 |
| D7S820 | 7 | 12 | D75820 | 7 | 12 |
| D13S317 | 11 | 11 | D13S317 | 11 | 11(A > T) |
| D5S818 | 11 | 11 | D5S818 | 11(T > C) | 11(T > C) |
| D2S441 | 14 | 15 | D2S441 | 14(C > T) | 15(C > T) |
| D19S433 | 14 | 15 | D19S433 | 14 | 15 |
| FGA | 24 | 26 | FGA | 24 | 26 |
| D18S51 | 12 | 15 | D18S51 | 12 | 15 |
| CSF1PO | 11 | 12 | CSF1PO | 11 | 12 |
| Penta D | 11 | 12 | Penta D | 11 | 12(A > G) |
| Penta E | 7 | 12 | Penta E | 7 | 12 |
| TH01 | 7 | 9.3 | TH01 | 7 | 9.3 |
| TPOX | 8 | 8 | TPOX | 8 | 8 |
| D10S1248 | 12 | 15 | D10S1248 | 12 | 15 |
| D22S1045 | 8 | 13 | D22S1045 | 8 | 13 |

5B. Sample 9947A

| | | | | | |
|---|---|---|---|---|---|
| SE33 | 19 | 29.2 | SE33 | 19(G > A) | 29.2 |
| D2S1338 | 19 | 23 | D2S1338 | 19(T > G) | 23(T > G, T > G) |
| vWA | 17 | 18 | vWA | 17 | 18 |
| D21S11 | 30 | 30 | D21S11 | 30(G > A) | 30(G > A) |
| D3S1358 | 14 | 15 | D3S1358 | 14(C > T) | 15(C > T) |
| D16S539 | 11 | 12 | D16S539 | 11 | 12 |
| D8S1179 | 13 | 13 | D8S1179 | 13 | 13(A > G) |
| D7S820 | 10 | 11 | D7S820 | 10(T > A) | 11 |
| D13S317 | 11 | 11 | D13S317 | 11 | 11 |
| D5S818 | 11 | 11 | D5S818 | 11(T > C) | 11(T > C) |
| D2S441 | 10 | 14 | D2S441 | 10(A > G) | 14(C > T) |
| D19S433 | 14 | 15 | D19S433 | 14 | 15 |
| FGA | 23 | 24 | FGA | 23 | 24 |
| D18S51 | 15 | 19 | D18S51 | 15 | 19 |
| CSF1PO | 10 | 12 | CSF1PO | 10 | 12 |
| Penta D | 12 | 12 | Penta D | 12 | 12 |
| Penta E | 12 | 13 | Penta E | 12 | 13 |
| TH01 | 8 | 9.3 | TH01 | 8 | 9.3 |
| TPOX | 8 | 8 | TPOX | 8 | 8 |
| D10S1248 | 13 | 15 | D10S1248 | 13 | 15 |
| D22S1045 | 8 | 11 | D22S1045 | 8 | 11 |

In a set of experiments, we evaluated the possibility of simultaneously amplifying all or a subset of these markers within a single PCR and analyzing the obtained multiplexes with ICEMS.

In a first set of experiments, an 8-plex was developed. Tables 6 and 7 compare and summarize data for the 8-plex experiments. Table 6 shows the eight target amplicon regions substantially simultaneously amplified and the genotyping of those markers. Table 7 summarizes the allele assignments obtained using embodiments of the methods of the present teaching with this multiplex amplification. Regarding the length information, ICEMS results were consistent with the CE results.

Table 6, beginning on page 21, line 1:

TABLE 6
Comparison of STR genotypes obtained from electrophoretic
sizing to an 8-plex ICEMS for sample #2.

| Electrophoretic sizing | | | ICEMS | | |
|---|---|---|---|---|---|
| marker | allele 1 | allele 2 | marker | allele 1 | allele 2 |
| vWA | 17 | 17 | vWA | 17 | 17(G > A) |
| D21S11 | 28 | 30.2 | D21S11 | 28 | 30.2 |
| D3S1358 | 15 | 16 | D3S1358 | 15(C > T) | 16(C > T) |
| D16S539 | 8 | 8 | D16S539 | 8 | 6 |
| D8S1179 | 10 | 13 | D8S1179 | 10 | 13(A > G) |
| D7S820 | 9 | 11 | D7S820 | 9 | 11 |
| D13S317 | 12 | 12 | D13S317 | 12(A > T) | 12(A > T) |
| D2S441 | 11.3 | 14 | D2S441 | 11.3 | 14(C > T) |

Table 7, beginning on page 21, line 5:

TABLE 7
Allele-assignment based on measured molecular masses
obtained from the 8-plex PCR-ICEMS assay.

| Measured | Best matching | | Single |
|---|---|---|---|
| mass | theoretical mass | Marker*allele | strand |
| 35171 | 35169.7 | D13S317*12(A > T) | forward |
| 36360 | 36359.6 | | reverse |
| 34095 | 34095.2 | D16S539*12 | forward |
| 33114 | 33113.3 | | reverse |
| 29056 | 29055.9 | D16S539*8 | forward |
| 28027 | 26270.2 | | reverse |
| 55004 | 55002.4 | D21S11*28 | forward |
| 55684 | 56683.7 | | reverse |
| 58041 | 58041.4 | D21S11*30.2 | forward |
| 59624 | 59820.8 | | reverse |
| 27893 | 27893 | D2S411*11.3 | forward |
| 28815 | 28814.7 | | reverse |
| 30364 | 30633.8 | D2S411*14(C > T) | forward |
| 31631 | 31631.5 | | reverse |
| 39420 | 39419.5 | D3S1358*15(C > T) | forward |
| 38916 | 38915.1 | | reverse |
| 40680 | 40679.3 | D3S1358*16(C > T) | forward |
| 40126 | 40125.8 | | reverse |
| 49588 | 49588.1 | D7S820*11 | forward |
| 49116 | 49115.8 | | reverse |
| 47070 | 47068.5 | D7S820*9 | forward |
| 46694 | 46694.2 | | reverse |
| 52003 | 52000.6 | D8S1179*10 | forward |
| 52267 | 52267.8 | | reverse |
| 55649 | 55849.0 | D8S1179*13(A > G) | forward |
| 56034 | 56032.2 | | reverse |
| 45783 | 45782.5 | vWA*17 | forward |
| 46759 | 46755.3 | | reverse |
| 45676 | 45766.5 | vWA*17(G > A) | forward |
| 46770 | 46770.3 | | reverse |

In a second set of experiments, a 14-plex was developed. Tables 8 and 9 compare and summarize data for the 14-plex experiments. In these experiments, 13 STRs and a sex determining marker (Amelogenin 1331) were characterized. Table 8 shows the fourteen target amplicon regions substantially simultaneously amplified and the genotyping of those markers. Table 9 summarizes the allele assignments obtained using embodiments of the methods of the present teaching with this multiplex amplification. Regarding the length information, ICEMS results were consistent with the CE results.

TABLE 8
Comparison of STR genotypes obtained from electrophoretic sizing to a
14-plex ICEMS for sample 9948.

| electrophoretic sizing | | | ICEMS | | |
|---|---|---|---|---|---|
| marker | allele 1 | allele 2 | marker | allele 1 | allele 2 |
| Amelogenin | X | Y | Amelogenin | X | Y |
| CSF1PO | 10 | 11 | CSF1PO | 10 | 11 |
| D10S1248 | 12 | 15 | D10S1248 | 12 | 15 |
| D13S317 | 11 | 11 | D13S317 | 11 | 11 |
| D16S539 | 11 | 11 | D16S539 | 11 | 11 |
| D21S11 | 29 | 30 | D21S11 | 29 | 30(G > A) |
| D22S1045 | 13 | 15 | D22S1045 | 13 | 15 |
| D2S441 | 11 | 12 | D2S441 | 11 | 12 |
| D3S1358 | 15 | 17 | D3S1358 | 15(C > T) | 17 |
| D5S818 | 11 | 13 | D5S818 | 11(T > C) | 13(T > C) |
| D7S820 | 11 | 11 | D7S820 | 11 | 11 |
| D8S1179 | 12 | 13 | D8S1179 | 12(A > G) | 13(A > G) |
| TPOX | 8 | 9 | TPOX | 8 | 9 |
| vWA | 17 | 17 | vWA | 17 | 17 |

Table 9, beginning on page 23, line 1:

TABLE 9
Allele-assignment based on measured molecular masses
obtained from the 14-plex PCR-ICEMS assay.

| Measured | Best matching | | Single |
|---|---|---|---|
| mass | theoretical mass | Marker*allele | strand |
| 32490 | 32490.9 | Amelogenin_X | forward |
| 32874 | 32875.3 | | reverse |
| 34397 | 34396.1 | Amelogenin_Y | forward |
| 34677 | 34677.4 | | reverse |
| 33177 | 33178.6 | CSF1PO*10 | forward |
| 32383 | 32383.1 | | reverse |
| 34437 | 34438.5 | CSF1PO*11 | forward |
| 33592 | 33593.8 | | reverse |
| 31419 | 31419.4 | D10S1248*12 | forward |
| 30191 | 30190.6 | | reverse |
| 35274 | 35273.9 | D10S1248*15 | forward |
| 33750 | 33750.8 | | reverse |
| 34373 | 34372.4 | D13S317*11 | forward |
| 35494 | 35495.2 | | reverse |
| 32836 | 32835.3 | D16S539*11 | forward |
| 31901 | 31902.5 | | reverse |
| 56717 | 56719.8 | D21S11*29 | forward |
| 58106 | 58109.6 | | reverse |
| 57913 | 57914.5 | D21S11*30(G > A) | forward |
| 59383 | 59384.5 | | reverse |
| 31684 | 31683.5 | D22S1045*13 | forward |
| 32077 | 32076.9 | | reverse |
| 33527 | 33526.7 | D22S1045*15 | forward |
| 33937 | 33938.1 | | reverse |
| 26986 | 26986.4 | D2S441*11 | forward |
| 27868 | 27888.1 | | reverse |
| 28196 | 28197.2 | D2S441*12 | forward |
| 29127 | 29127.9 | | reverse |
| 39420 | 39419.5 | D3S1358*15(C > T) | forward |
| 38913 | 38915.1 | | reverse |
| 41923 | 41924.1 | D3S1358*17 | forward |
| 41349 | 41352.6 | | reverse |
| 38409 | 38409.9 | D5S818*11(T > C) | forward |
| 37500 | 37500.3 | | reverse |
| 40930 | 40929.6 | D5S818*13(T > C) | forward |
| 39923 | 39921.8 | | reverse |
| 49586 | 49588.1 | D7S820*11 | forward |
| 49114 | 49115.8 | | reverse |
| 54857 | 54857.6 | D8S1179*12(A > G) | forward |
| 55191 | 55191.8 | | reverse |
| 58067 | 56066.3 | D8S1179*13(A > G) | forward |
| 56453 | 56451.6 | | reverse |
| 23941 | 23941.5 | TPOX*8 | forward |
| 23683 | 23682.3 | | reverse |
| 25200 | 25201.3 | TPOX*9 | forward |
| 24894 | 24893.1 | | reverse |
| 46288 | 46289.1 | vWA*17 | forward |
| 46922 | 46921.4 | | reverse |

It is evident from the above examples that an improved method of STR-typing is provided by the subject invention. STR-typing is traditionally accomplished via selective amplification using PCR followed by capillary electrophoresis. However, capillary electrophoresis-based techniques can be time consuming to perform and provide little information beyond fragment length. Moreover, STR amplicons contain more discriminative information than just the fragment length. For example, experiments on selected STR-alleles by direct sequencing analysis (A. Urquhart et al. (1994) Int. J. Legal Med. 107: 13-20; B. Rolf et al. (1997) Int. J. Legal Med. 110: 69-72; P. Grubwieser et al. (2005) Int. J. Legal Med. 119: 164-166; P. Grubwieser et al. (2007) Int. J. Legal Med. 121: 85-89) have classified STRs as “simple” (repeats that contain only units of identical length and sequence), “compound” (repeats that comprise two or more adjacent simple repeats), or “complex” (repeats that contain several repeat blocks of variable unit lengths along with more or less variable intervening sequences), indicating that there is additional sequence variability in STRs that could allow for discrimination of fragments with identical length. A method that allows for discrimination of fragments with identical length will be beneficial, for example, for a number of forensic applications such as the identification of remains or samples that have been exposed to environmental conditions such as high temperatures (e.g. fire) or moisture that cause heavy degradation of DNA. What is provided herein is a method that is capable of discriminating sequence differences in STR amplicons to allow for such discrimination of fragments.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for the determination of nucleic acid length and sequence variation, comprising the steps of:
(a) amplifying at least two or more specific regions of an oligonucleotide molecule in a sample comprising oligonucleotide molecules to produce a sample of amplicons, the sample of amplicons comprising two or more different amplicons;
(b) denaturing the amplicons in the sample of amplicons to produce a first set of single-stranded amplicons and a second set of single-stranded amplicons, single-stranded amplicons of the second set being complementary to the corresponding single-stranded amplicons of the first set;
(c) subjecting the first and second set of single-stranded amplicons to mass spectrometric analysis to obtain the masses of the amplicons in the first and second set of single-stranded amplicons; and
(d) determining the length and nucleic acid sequence variation of a specific amplified region by comparing the masses of the first and second set of amplicons to the mass of a reference nucleic acid sequence.

2. The method of claim 1, wherein step (a) comprises amplifying at least four or more specific regions of an oligonucleotide molecule in a sample comprising oligonucleotide molecules to produce a sample of amplicons.

3. The method of claim 1, wherein step (a) comprises amplifying at least eight or more specific regions of an oligonucleotide molecule in a sample comprising oligonucleotide molecules to produce a sample of amplicons.

4. The method of claim 1, wherein in step (a) the amplification is selected to produce amplicons having less than about 250 base pairs.

5. The method of claim 4, wherein in step (a) the amplification is selected to produce amplicons having less than about 100 base pairs.

6. The method of claim 1, wherein step (b) comprises: loading at least a portion of the sample of amplicons on a chromatographic column; and heating the chromatographic column to denature the amplicons.

7. The method of claim 1, wherein step (c) is conducted using an electrospray ionization mass spectrometry instrument.

8. The method of claim 1, further comprising distinguishing between alleles having identical amplicon length based at least on the nucleic acid sequence variation determined in step (d).

9. The method of claim 1, wherein step (d) comprises determining variation between amplicons of the same specific region of the oligonucleotide molecule.

10. The method of claim 1, wherein the variation determined in step (d) comprises a single nucleotide polymorphism (SNP).

11. The method of claim 1, wherein the variation determined in step (d) comprises a short tandem repeat variation (STR).

12. The method of claim 1, wherein the variation determined in step (d) comprises a single nucleotide polymorphism short tandem repeat variation (SNPSTR).

13. The method of claim 1, wherein the oligonucleotide comprises deoxyribonucleic acid (DNA) or a fragment thereof.

14. The method of claims 13, wherein the oligonucleotide comprises one or more of mitochondrial DNA, nuclear DNA, bacterial DNA, viral DNA, or a fragment thereof.

15. A method for the determination of nucleic acid length and sequence variation, comprising the steps of:
(a) amplifying a specific region of an oligonucleotide molecule in a sample comprising oligonucleotide molecules to produce a sample of amplicons, the sample of amplicons comprising two or more different amplicons;
(b) denaturing the amplicons in the sample of amplicons to produce a first set of single-stranded amplicons and a second set of single-stranded amplicons, single-stranded amplicons of the second set being complementary to the corresponding single-stranded amplicons of the first set;
(c) subjecting the first and second set of single-stranded amplicons to mass spectrometric analysis to obtain the masses of the amplicons in the first and second set of single-stranded amplicons;
(d) determining the length and sequence variation of the specific amplified region of the oligonucleotide molecule by comparing the masses of the first and second set of amplicons to the mass of a reference nucleic acid sequence.

16. The method of claim 15, wherein step (a) comprises amplifying at least four or more specific regions of an oligonucleotide molecule in a sample comprising oligonucleotide molecules to produce a sample of amplicons.

17. The method of claim 15, wherein step (a) comprises amplifying at least eight or more specific regions of an oligonucleotide molecule in a sample comprising oligonucleotide molecules to produce a sample of amplicons.

18. The method of claim 15, wherein in step (a) the amplification is selected to produce amplicons having less than about 250 base pairs.

19. The method of claim 18, wherein in step (a) the amplification is selected to produce amplicons having less than about 100 base pairs.

20. The method of claim 15, wherein step (b) comprises:
loading at least a portion of the sample of amplicons on a chromatographic column; and heating the chromatographic column to denature the amplicons.

21. The method of claim 15, wherein step (c) is conducted using an electrospray ionization mass spectrometry instrument.

22. The method of claim 15, further comprising distinguishing between alleles having identical amplicon length based at least on the nucleic acid sequence variation determined in step (d).

23. The method of claim 15, wherein step (d) comprises determining variation between amplicons of the same specific region of the oligonucleotide molecule.

24. The method of claim 15, wherein the variation determined in step (d) comprises a single nucleotide polymorphism (SNP).

25. The method of claim 15, wherein the variation determined in step (d) comprises a short tandem repeat variation (STR).

26. The method of claim 15, wherein the variation determined in step (d) comprises a single nucleotide polymorphism short tandem repeat variation (SNPSTR).

27. The method of claim 15, wherein the oligonucleotide comprises deoxyribonucleic acid (DNA) or a fragment thereof.

28. The method of claim 27, wherein the oligonucleotide comprises one or more of mitochondrial DNA, nuclear DNA, bacterial DNA, viral DNA, or a fragment thereof.

29. A method for the determination of nucleic acid length and sequence variation, comprising the steps of:
(a) amplifying at least two or more specific regions of an oligonucleotide molecule in a sample comprising oligonucleotide molecules to produce a sample of amplicons, the sample of amplicons comprising two or more different amplicons;
(b) denaturing the amplicons in the sample of amplicons to produce a first set of single-stranded amplicons and a second set of single-stranded amplicons, single-stranded amplicons of the second set being complementary to the corresponding single-stranded amplicons of the first set;
(c) subjecting the first and second set of single-stranded amplicons to mass spectrometric analysis to obtain the masses of the amplicons in the first and second set of single-stranded amplicons;
(d) generating a list of paired candidate sequences based on masses of the first and second set of amplicons, one member of the pair corresponding to a candidate sequence for the first set of amplicons and the other member of the pair corresponding to a candidate sequence for the second set of amplicons, and where the sequence pairs are complimentary; and
(e) determining the length and nucleic acid sequence variation of a specific amplified region by comparing the candidate sequences to a reference nucleic acid sequence.

30. The method of claim 29, wherein step (a) comprises amplifying at least four or more specific regions of an oligonucleotide molecule in a sample comprising oligonucleotide molecules to produce a sample of amplicons.

31. The method of claim 29, wherein step (a) comprises amplifying at least eight or more specific regions of an oligonucleotide molecule in a sample comprising oligonucleotide molecules to produce a sample of amplicons.

32. The method of claim 29, wherein in step (a) the amplification is selected to produce amplicons having less than about 250 base pairs.

33. The method of claim 32, wherein in step (a) the amplification is selected to produce amplicons having less than about 100 base pairs.

34. The method of claim 29, wherein step (b) comprises:
loading at least a portion of the sample of amplicons on a chromatographic column; and heating the chromatographic column to denature the amplicons.

35. The method of claim 29, wherein step (c) is conducted using an electrospray ionization mass spectrometry instrument.

36. The method of claim 29, further comprising distinguishing between alleles having identical amplicon length based at least on the nucleic acid sequence variation determined in step (e).

37. The method of claim 29, wherein step (e) comprises determining variation between amplicons of the same specific region of the oligonucleotide molecule.

38. The method of claim 29, wherein the variation determined in step (d) comprises a single nucleotide polymorphism (SNP).

39. The method of claim 29, wherein the variation determined in step (d) comprises a short tandem repeat variation (STR).

40. The method of claim 29, wherein the variation determined in step (d) comprises a single nucleotide polymorphism short tandem repeat variation (SNPSTR).

41. The method of claim 29, wherein the oligonucleotide comprises deoxyribonucleic acid (DNA) or a fragment thereof.

42. The method of claim 41, wherein the oligoliucleotide comprises one or more of mitochondrial DNA, nuclear DNA, bacterial DNA, viral DNA, or a fragment thereof.
